SEQUENCE LISTING (17 sequences)

SEQ ID NO: 1 - protein, 426 aa, Zea mays
MIGCAKPLAAPLQAWARPPSPAGRRLPPSFCAPDTSPALTRAVESPGQSQSDDAPPPRSGEAASSLAPRASSHLDRWSRSRALRSGHRPALNRAALSSASVSAPPVIKSPRPEDAAVAAEDGEDDDVCEAERDAAAGKAIYIVSDGTGWTAEHSVNAALGQFENCLADRGCAVNTHLFSLIDDMDRLIEVIKQAAKEGALVLYTLADPSMAEATKKACDFWGVPCTDVLRPTVEAIASHIGVAPSGIPRSFPSRNGRLSEDYFQRIDAIDFTIKQDDGVLPQNFYRADIVLAGVSRTGKTPLSIYLAQKGYKVANVPIVMGVALPKSLFEINQDKVFGLTINPAILQGIRKTRAKTLGFDGRQSNYAEMDHVRQELVHANQIFVQNPWWPVIAVTGKAIEETAAVILGILHDRKQKCSMPRISKRY

SEQ ID NO: 2 - protein, 419 aa, Sorghum bicolor
MIGSAKPLAVAAPLQPPSPAGRRLAPLFCAPDSSPALTRAVESPGQSQSDGAPPPRPDEVASSLTRRASPQLSRWSRLRTLRSNRRPGLDRGVLSSASSSASAPPVTKTSRPEDGAVAVEDGEDDDVPGGKSIYIVSDGTGWTAEHSVNAALGQFEHCFVDRGCAVNTHLFSMINDMDRILEVIKQAAKEGALVLYTLADPSMAESAKKACDFWGVPSTDVLRPTVEAIASHMGVAPSGIPRSSPSRPCQLTEDYFRRIDAIDFTIKQDDGAQPENLNRADIVLAGVSRTGKTPLSIYLAQKGYKVANVPIVMGVNLPKTLFEISQDKVFGLTINPVVLQAIRKTRAKALGFADGQSNYAEMDHVRQELLHANQIFAQNPMWPVIAVTGKAIEETAAVVVRIYLDRKQKYSMPRISKRY

SEQ ID NO: 3 - protein, 429 aa, Setaria italica
MIGAGANPLAAPLLGPPSPAGRRLAPGPVSCALDPSPVLAPAAAEAPGQSDRAPPPRAPDDAASSSSSSSRALKASSQLSRWSRGRALRSGRRLRLVDRAAVSSAPPPPVTKPPPQPQQPPSLLYDDTPAVAPEDDDDVCEAERDAAAGKAIYLVSDGTGWTAEHSVNAALGQFEHCLVDSGCSVSTHLFSGLIKQAAKEGALVLYTLADPSMAETTKKACDFWGVPSTDVLRPTVEAIANHIGVAPSGIPRSSPSRQGKLTEDYFRRIDAIDFTIKQDDGAQPQNLNRADIVLVGVSRTGKTPLSIYLAQKGYKVANVPIVMGINLPKALFEINQDKIFGLTINPVVLQAIRKTRAKTLGFDGHKSNYAEMEHVRQELAHANQIFAQNPVWPVIVVTGKAVEETAAVVVRILHDKKQKCSMPRISKRY

SEQ ID NO: 4 - protein, 433 aa, Zea mays
MAAAFSSTVGAPASTPTRSSFLGKKLNKPQVSAAVTYHGKSSSSNSRFKAMAAKEVDETKQTDEDRWKGLAYDISDDQQDITRGKGLVDNLFQAPMGDGTHVAVLSSYDYISQGQKSYNFDNMMDGFYIAKGFMDKLVVHLSKNFMTLPNIKVPLILGIWGGKGQGKSFQCELVFAKMGITPIMMSAGELESGNAGEPAKLIRQRYREASDLIKKGKMSCLFINDLDAGAGRMGGTTQYTVNNQMVNATLMNIADNPTNVQLPGMYNKEDNPRVPIIVTGNDFSTLYAPLIRDGRMEKFYWAPTREDRIGVCKGIFRTDGVDEEHVVQLVDTFPGQSIDFFGALRARVYDDEVRRWVSETGVENIARKLVNSKEGPPTFEQPKITIEKLLEYGHMLVAEQENVKRVQLADKYLNEAALGEANEDAMKTGSFFK

SEQ ID NO: 5 - protein, 473 aa, Sorghum bicolor
MAAAFSSTSVVVAVPVTTSTAANSFLGSNNNNKQKLTVSSIRKKKQHQQGGRGLIIIRAMAAVNKQEVDETKQTEQDRWRGLAYDTSDDQQDITRGKGMIDSLFQAPMGDGTHVAVLSSYDYISQGLRQYNTMDGYYIAPAFMDKLVLHIAKNFMTLPNIKVPLILGIWGGKGQGKSFQCELVFAKMGINPIVMSAGELESGNAGEPAKLIRQRYREAADLISKGKMSCLFINDLDAGAGRMGGTTQYTVNNQMVNATLMNIADNPTNVQLPGMYSKVDNPRVPIIVTGNDFSTLYAPLIRDGRMDKFYWAPTRDDRIGVCKGIFRTDGVPDEHVVQLVDAFPGQSIDFFGALRARVYDDEVRRWVAETGVENIARRLVNSKEAPPTFEQPRMTLDKLMEYGRMLEEEQENVKRVQLADKYLTEAALGDANDFYGKAAQQVHVPVPVPEGCTDPRAGNFDPVARSDDGSCVYN

SEQ ID NO: 6 - protein, 431 aa, Panicum virgatum
MAAAFSSTVGAPASTPASFLGSKLSRKQATAAAVNYHGKSSGANRFRVMAKEVDESKQTDSDRWKGLAYDISDDQQDITRGKGLVDSLFQAPTGDGTHEAVLSSYEYLSQGLRDYSAWDNMKDGFYIAPAFMDKLVVHLSKNFMTLPNIKVPLILGIWGGKGQGKSFQCELVFAKMGITPIMMSAGELESGNAGEPAKLIRQRYREAADIIKKGKMCCLFINDLDAGAGRMGGTTQYTVNNQMVNATLMNIADNPTNVQLPGMYNKEENPRVPIVVTGNDFSTLYAPLIRDGRMEKFYWAPTREDRIGVCKGIFRTDGVPDEDVVKLVDTFPGQSIDFFGALRARVYDDEVRKWVAETGVENIGRKLVNSKEGPPKFEQPKITIAKLLEYGHMLVAEQENVKRVQLADKYLSEAALGEANEDAMKTGAFFK

SEQ ID NO: 7 - protein, 430 aa, Setaria italica
MAAAFSSTVGAPASTPASFLGKKLRTQATAAVNYHVKSSSANRFKVMAAKDVDETKQTDKDRWRGLVNDISDDQQDITRGKGFVDSLFQAPMGDGTHEAVLSSYEYLSQGLRDYTGWDNMKDGFYIAPAFMDKLVVHLSKNFMTLPNIKVPLILGIWGGKGQGKSFQCELVFAKMGITPIMMSAGELESGNAGEPAKLIRQRYREAADIIKKGKMCCLFINDLDAGAGRMGGTTQYTVNNQMVNATLMNIADNPTNVQLPGMYNKEENPRVPIVVTGNDFSTLYAPLIRDGRMEKFYWAPTREDRIGVCKGIFRTDGVPEADVVKLVDTFPGQSIDFFGALRARVYDDEVRKWVAETGIENIGKKLVNSKDGPPTFDQPKMTIEKLLEYGHMLVEEQENVKRVQLADKYLSEAALGEANEDAMKTGAFFK

SEQ ID NO: 8 - protein, 947 aa, Zea mays
MAASVSRAICVQKPGSKCTRDREATSFARRSVAAPRPPHAKAAGVIRSDSGAGRGQHCSPLRAVVDAAPIQTTKKRVFHFGKGKSEGNKTMKELLGGKGANLAEMASIGLSVPPGFTVSTEACQQYQDAGCALPAGLWAEIVDGLQWVEEYMGATLGDPQRPLLLSVRSGAAVSMPGMMDTVLNLGLNDEVAAGLAAKSGERFAYDSFRRFLDMFGNVVMDIPRSLFEEKLEHMKESKGLKNDTDLTASDLKELVGQYKEVYLSAKGEPFPSDPKKQLELAVLAVFNSWESPRAKKYRSINQITGLRGTAVNVQCMVFGNMGNTSGTGVLFTRNPNTGEKKLYGEFLVNAQGEDVVAGIRTPEDLDAMKNLMPQAYDELVENCNILESHYKEMQDIEFTVQENRLWMLQCRTGKRTGKSAVKIAVDMVNEGLVEPRSAIKMVEPGHLDQLLHPQFENPSAYKDQVIATGLPASPGAAVGQVVFTAEDAEAWHSQGKAAILVRAETSPEDVGGMHAAVGILTERGGMTSHAAVVARWWGKCCVSGCSGIRVNDAEKLVTIGSHVLREGEWLSLNGSTGEVILGKQPLSPPALSGDLGTFMAWVDDVRKLKVLANADTPDDALTARNNGAQGIGLCRTEHMFFASDERIKAVRQMIMAPTLELRQQALDRLLTYQRSDFEGIFRAMDGLPVTIRLLDHPSYEFLPEGNIEDIVSELCAETGANQEDALARIEKLSEVNPMLGFRGCRLGISYPELTEMQARAIFEAAIAMTNQGVQVFPEIMVPLVGTPQELGHQVTLIRQVAEKVFANVGKTIGYKVGTMIEIPRAALVADEIAEQAEFFSFGTNDLTQMTFGYSRDDVGKFIPVHLAQGILQHDPFEVLDQRGVGELVKFATERGRKARPNLKVGICGEHGGEPSSVAFFAKAGLDFVSCSPFRVPIARLAAAQVLV

SEQ ID NO: 9 - protein, 476 aa, Sorghum bicolor
MSPQTETKASVGFKAGVKDYKLTYYTPEYETKDTDILAAFRVTPQLGVPPEEAGAAVAAESSTGTWTTVWTDGLTSLDRYKGRCYHIEPVPGDPDQYICYVAYPLDLFEEGSVTNMFTSIVGNVFGFKALRALRLEDLRIPPAYVKTFQGPPHGIQVERDKLNKYGRPLLGCTIKPKLGLSAKNYGRACYECLRGGLDFTKDDENVNSQPFMRWRDRFVFCAEAIYKAQAETGEIKGHYLNATAGTCEEMIKRAVFAKELGVPIVMHDYLTGGFTANTTLSHYCRDNGLLLHIHRAMHAVIDRQKNHGMHFRVLAKALRMSGGDHIHSGTVVGKLEGEREITLGFVDLLRDDFIEKDRSRGIFFTQDWVSMPGVIPVASGGIHVWHMPALTEIFGDDSVLQFGGGTLGHPWGNAPGAAANRVALEACVQARNEGRDLAREGNEIIKAACKWSAELAAACEIWKEIKFDTFKAMDTL

SEQ ID NO: 10 - protein, 476 aa, Saccharum officinarum; identical to SEQ ID NO: 9

SEQ ID NO: 11 - protein, 476 aa, Miscanthus x giganteus; identical to SEQ ID NO: 9

SEQ ID NO: 12 - protein, 477 aa, Panicum virgatum
MSPQTETKASVGFKAGVKDYKLTYYTPEYETKDTDILAAFRVTPQPGVPPEEAGAAVAAESSTGTWTTVWTDGLTSLDRYKGRCYHIEPVPGEADQYICYIAYPLDLFEEGSVTNMFTSIVGNVFGFKALRALRLEDLRIPPAYSKTFQGPPHGIQVERDKLNKYGRPLLGCTIKPKLGLSAKNYGRACYECLRGGLDFTKDDENVNSQPFMRWRDRFVFCAEAIYKAQAETGEIKGHYLNATAATCEEMIKRAVFARELGVPIVMHDYITGGFTANTSLAHYCRDNGLLLHIHRAMHAVIDRQKNHGMHFRVLAKALRMSGGDHIHAGTVVGKLEGEREITLGFVDLLRDDFIEKDRSRGIFFTQDWVSMPGVIPVASGGIHVWHMPALTEIFGDDSVLQFGGGTLGHPWGNAPGAAANRVALEACVQARNEGRDLAREGNEIIKAACKWSPELAAACEVWKAITFDFAPVDTIDK

SEQ ID NO: 13 - protein, 476 aa, Setaria italica
MSPQTETKASVGFKAGVKDYKLTYYTPEYETKDTDILAAFRVTPQPGVPPEEAGAAVAAESSTGTWTTVWTDGLTSLDRYKGRCYHIEPVPGEADQYICYIAYPLDLFEEGSVTNMFTSIVGNVFGFKRSRALRLEDLRIPPAYAKTFQGPPHGIQVERDKLNKYGRPLLGCTIKPKLGLSAKNYGRACYECLRGGLDFTKDDENVNSQPFMRWRDRFVFCAEAIYKAQAETGEIKGHYLNATAGTCEEMIKRAAFARELGVPIVMHDYLTGGFTANTSLSYYCRDNGLLLHIHRAMHAVIDRQKNHGMHFRVLAKALRMSGGDHIHSGTVVGKLEGEREITLGFVDLLRDDFIEKDRSRGIFFTQDWASMPGVIPVASGGIHVWHMPALTEIFGDDSVLQFGGGTLGHPWGNAPGTAANRVALEACVQARNEGRDLAREGNEIIKAACKWSPELAAACEVWKEIKFEGSKAMDTL

SEQ ID NO: 14 - protein, 420 aa, Zea mays
MIGSTKPLAAPLHPPYPSGRRLAPPSCAPDSSPALTPAVERPGQSQSDGAPPPPRPDEVASSLALRASPQLNRWSRSRALRSNRRPGLESALSSSSAASVTKTSRPEDAAVAVEDGEDDDVCVETDAAGGKAIYIVSDGTGWTAEHSVNAALGQFEHCFVDRGCAVNTHLFSMIDNMDRLLEVIKQAAKEGALVLYTLADPSMAESTKKACDFWGVPSTDVLRPTVEAIASHMGVAPSGIPRSSPSRQGQLTEDYFRRIDAIDFTIKQDDGALPQNLNRADIVLVGVSRTGKTPLSIYLAQKGYKVANVPVVMGVDLPKTLFEINQDKVFGLTINPVVLQAIRKTRAKALGFGDGYQSNYAEMDHVRQELLHANQIFAQHPMWPVIAVTGRAIEETAAVVVRILQDRIQKYSMPRISKRY

SEQ ID NO: 15 - protein, 429 aa, Sorghum bicolor
MYLYTASASSMIGSAKPLAWAPLQARPPSTAGRRLAPSFCAPDTAPALQSQSDDGAPPPRPDETATSPALLRSSQLSRWSRSRGLRSGRRVGLDRAALSSASAPPVTKPSRPEDAAVAVEDGEDDDDDVCEAERDDAAGKAIYIVSDGTGWTAEHSVNAALGQFENCIADRGCAVNTHLIPLIDSMDQLLDVIKQAAKEGALVLYTLADPSMAEAAKKACDFWGVPSTDVLRPTVEAIASHIGVAPSGIPRSSPSRKGQLTEDYFQRIDAIDFTIKQDDGAQPQNLKRADIVLAGVSRTGKTPLSMYLAQKGYKVANVPIVMGVKLPKSLFEINQDKIFGLTINPVVLQAIRKTRAKALGFADGYQSNYAEMEHVRQDLAHANQIFAENPRWPVIAVTGKAIEETAAIIVRILQDRKEKCSMPRISKRY

SEQ ID NO: 16 - protein, 423 aa, Panicum hallii
MIGGAKPLAAPLLGATPPAGRRLATAACAPDPSPALATAAAQSPGQSDRAPPPRPPDESAASSTALRGTSQLSRWSRARALRSGRRLGLDRAAVSLAPPTMPPPTPSLVPDVAAGAAEDDDDDLCDAERDAVAGKAIYMVSDGTGWTAEHSVNAALGQFEHCLVDRECSVNTHLFSGIDDMDRLLEVIKQAAKEGALVLYTLADPSMAEATKKACDFWGVPCTDVLRPTVEAIAAHIGVAPSGIPRSSPSRKGQLTEDYFRRIEAIDFTIKQDDGAQPQNLNRADIVLVGVSRTGKTPLSIYLAQKGYKVANVPIVMGVNLPKALFEINQDKIFGLTINPVILQAIRKTRAKTLGFDGYTSNYAEMAHVRQELDHANQIFAQNPMWPVIGVTGKAIEETAAVVVRVYHDRKQKCSMPRISKRY

SEQ ID NO: 17 - protein, 454 aa, Panicum miliaceum
MIGGAKPLAAPLLGATPPAGRRLATAACAPDPSPALATAAAQNPGQSDRAPPPRPPDESAASSTALRGTSQLSRWSRARALRSGRRLGLDRAAVSSAPPVTRPPTMPTPTPSLVPEVAAGAAEDDDDDLCDAERDAVAGKAIYMVSDGTGWTAEHSVNAALGQFEHCLVDRECSVNTHLFSGIDDMDRLLEVIKQAAKEGALVLYTLADPSMAEATKKACDFWGVPCTDVLRPTVEAIAAHIGVAPSGIPRSSPSRKGQLTEDYFRRIEAIDFTIKQDDGAQPQNLNRADIVLVGVSRTGKTPLSIYLAQKGYKVANVPIVMGVNLPKALFEINQDKIFGLTINPVILQAIRKTRAKTLGFDGYTSNYAEMAHVRQELDHANQIFAQNPMWPVIGVTGKAIEETAAVVVRVYHDRKQKCSMPRISKRVAPVLVYDYRQVVNTHVEYQKVFAKEY

(19) United States
(12) Patent Application Publication
(10) Pub. No.: US 2024/0240196 A1
(43) Pub. Date: Jul. 18, 2024

(54) C4 PLANTS WITH INCREASED PHOTOSYNTHETIC EFFICIENCY

(71) Applicant: The Board of Trustees of the University of Illinois, Urbana, IL (US)

(72) Inventors: Stephen P. LONG, Urbana, IL (US); Yu WANG, Urbana, IL (US); Kher Xing CHAN, Urbana, IL (US)

(73) Assignee: The Board of Trustees of the University of Illinois, Urbana, IL (US)

(21) Appl. No.: 18/563,824

(22) PCT Filed: May 26, 2022
(86) PCT No.: PCT/US2022/031036

§ 371 Date: Nov. 22, 2023

Related U.S. Application Data
(60) Provisional application No. 63/193,566, filed on May 26, 2021.

(51) Int. Cl. C12N 15/82; C12N 9/12; C12N 9/88

(52) U.S. Cl. CPC C12N 15/8269; C12N 9/1294; C12N 9/88; C12N 15/8213; C12Y 207/09001; C12Y 401/01039

(57) ABSTRACT

Aspects of the present disclosure relate to genetically altered plants with increased activity of one or more of a PPDK regulatory protein (PDRP), a Rubisco activase (Rea) protein, or a Rubisco protein that have increased photosynthetic efficiency under fluctuating light conditions. Further, aspects of the present disclosure relate to methods of producing and cultivating the genetically altered plants of the present disclosure.

### CROSS-REFERENCE TO RELATED APPLICATIONS

This application claims the benefit of U.S. Provisional Application No. 63/193,566, filed May 26, 2021, which is hereby incorporated by reference in its entirety.

This invention was made with government support under Award Number DE-SC0018420 awarded by DOE Center for Advanced Bioenergy and Bioproducts Innovation (U.S. Department of Energy, Office of Science, Office of Biological and Environmental Research). The government has certain rights in the invention.

### STATEMENT REGARDING FEDERALLY-SPONSORED RESEARCH

### Submission of Sequence Listing as Ascii Text File

The content of the following submission on ASCII text file is incorporated herein by reference in its entirety: a computer readable form (CRF) of the Sequence Listing (file name: 794542001640SEQLIST.TXT, date recorded: May 23, 2022, size: 67,822 bytes).

### TECHNICAL FIELD

The present disclosure relates to genetically altered plants. In particular, the present disclosure relates to genetically altered plants with increased activity of one or more of a PPDK regulatory protein (PDRP), a Rubisco activase (Rca) protein, or a Rubisco protein that have increased photosynthetic efficiency under fluctuating light conditions. Further, the present disclosure relates to methods of producing and cultivating the genetically altered plants of the present disclosure.

### BACKGROUND

The yield potential of a given genotype at a given location is the product of the incident photosynthetically active radiation over the growing season, the efficiency of the crop in intercepting that radiation (εᵢ), the efficiency of conversion of intercepted radiation into plant mass (ε_{c}) and the efficiency of partitioning that mass into the harvested product (εₚ), also termed harvest index. Plant breeding has optimized s, and ep to points where there is little opportunity for further improvement in the major crops (Zhu, X.-G. et al. (2010) Improving photosynthetic efficiency for greater yield. Annual review of plant biology, 61, 235-261; Long, S. P., Burgess, S. and Causton, I. (2019) Redesigning crop photosynthesis. Sustaining Global Food Security: The Nexus of Science and Policy, 128). By contrast, the third factor, & (also known as light use efficiency) of C3 and C4 crops, governed by photosynthesis, falls well below its theoretical maximum (Zhu, X.-G., Long, S. P. and Ort, D. R. (2008) What is the maximum efficiency with which photosynthesis can convert solar energy into biomass? Current Opinion in Biotechnology, 19, 153-159). Multiple theoretical analysis and genetic engineering studies have shown that there is considerable potential to increase photosynthetic efficiency in both C3 and C4 crops (Murchie, E. et al. (2009) Agriculture and the new challenges for photosynthesis research. New Phytologist, 181, 532-552; Kromdijk, J. et al. (2016) Improving photosynthesis and crop productivity by accelerating recovery from photoprotection. Science, 354, 857-861; Salesse-Smith, C. E. et al. (2018) Overexpression of Rubisco subunits with RAF1 increases Rubisco content in maize. Nature Plants, 4, 802-810; Long, S. P. et al. (2019) Redesigning crop photosynthesis. Sustaining Global Food Security: The Nexus of Science and Policy, 128; South, P. F. et al. (2019) Synthetic glycolate metabolism pathways stimulate crop growth and productivity in the field. Science, 363; López-Calcagno, P. E. et al. (2020) Stimulating photosynthetic processes increases productivity and water-use efficiency in the field. Nature Plants, 6, 1054-1063; Yoon, D.-K. et al. (2020) Transgenic rice overproducing Rubisco exhibits increased yields with improved nitrogen-use efficiency in an experimental paddy field. Nature Food, 1, 134-139). While increasing stress tolerance of crops is another important route to increasing productivity, experience shows that increasing genetic yield potential, can address both by increasing average achieved yields under both optimal and stress conditions (Wu, J. R et al. (2019) Overexpression of zmm28 increases maize grain yield in the field. Proceedings of the National Academy of Sciences U.S.A, 116, 23850-23858). For example, a detailed analysis of progressive gains in yield potential through soybean breeding showed that these have resulted in achieved yield increases in years with both good and sub-optimal production conditions (Koester, R P. et al. (2014) Historical gains in soybean (Glycine max Merr.) seed yield are driven by linear increases in light interception, energy conversion, and partitioning efficiencies. Journal of Experimental Botany, 65, 3311-3321).

Photosynthesis is studied predominantly at steady-state in saturating light. In the field, however, leaves are rarely in steady-state, and are subject to frequent fluctuations in light intensity. While light may change in a second, adjustment of photosynthesis may take several minutes, leading to inefficiencies. A great deal of progress has been made towards understanding the dynamic response to light in C3 plants, and primary factors affecting the non-steady-state photosynthesis rate of C3 plants have been identified (although the major limitations vary with species).

For C4 plants, energy use efficiency limitations of C4 photosynthesis under steady-state conditions have been analyzed via a number of empirical analyses and biochemical models (Laisk, A. and Edwards, G. E. (2000) A mathematical model of C4 photosynthesis: the mechanism of concentrating CO2 in NADP-malic enzyme type species. Photosynthesis Research, 66, 199-224; Bellasio, C. and Griffiths, H. (2014) The operation of two decarboxylases, transamination, and partitioning of C4 metabolic processes between mesophyll and bundle sheath cells allows light capture to be balanced for the maize C4 pathway. Plant Physiology, 164, 466-480; Wang, Y. et al. (2014) Three distinct biochemical subtypes of C4 photosynthesis? A modelling analysis. Journal of Experimental Botany, 65, 3567-3578; Wang, Y. et al. (2014) Elements Required for an Efficient NADP-Malic Enzyme Type C4 Photosynthesis. Plant Physiology, 164, 2231-2246; Yin, X. and Struik, P. C. (2018) The energy budget in C4 photosynthesis: insights from a cell-type-specific electron transport model. New Phytologist, 218, 986-998; Yin, X. and Struik, P. C. (2021) Exploiting differences in the energy budget among C4 subtypes to improve crop productivity. New Phytologist, 229, 2400-2409). However, no mechanistic modeling studies have analyzed limitations of non-steady-state C4 photosynthesis. Major food and fiber C4 crops, such as maize, sorghum, sugarcane, and Miscanthus, predominantly utilize the NADP-ME form of C4 photosynthesis. Despite high productivities, these crops fall well short of the theoretical maximum solar conversion efficiency of 6%. Understanding the basis of these inefficiencies is key to achieving bioengineering and breeding strategies to increase sustainable productivity of these C4 crops.

There exists a clear need to identify the primary factors affecting the non-steady-state photosynthesis rate of C4 plants. These factors can be used in bioengineering and breeding strategies to improve the efficiency of non-steady-state photosynthesis C4 crop plants.

### BRIEF SUMMARY

In order to meet these needs, the present disclosure provides a dynamic model, which was developed to predict the potential limitations within C4 photosynthesis in fluctuating light, and to suggest feasible targets to improve energy use efficiency of C4 crops. The model output results provide the major factors limiting photosynthesis during dark-to-high-light transitions for these crops, namely Rubisco activase (Rca), PPDK regulatory protein, and stomatal conductance. Bioengineering and/or breeding C4 crops to address these limitations will improve the photosynthetic efficiency of these crops.

An aspect of the disclosure includes a genetically altered plant or plant part including one or more first genetic alterations that increase activity of a PPDK regulatory protein (PDRP), as compared to a wild type plant or plant part grown under the same conditions, wherein the genetically altered plant is a C4 plant.

A further aspect of the disclosure includes a genetically altered plant or plant part including one or more first genetic alterations that increase activity of a Rubisco activase (Rca) protein and/or a Rubisco protein, as compared to a wild type plant or plant part grown under the same conditions, wherein the genetically altered plant is a C4 plant.

An additional embodiment of this aspect, which may be combined with any of the preceding embodiments that has one or more first genetic alterations that increase activity of a PPDK regulatory protein (PDRP), further includes one or more second genetic alterations that increase activity of a Rubisco activase (Rca) protein and/or a Rubisco protein, as compared to a wild type plant or plant part grown under the same conditions. Still another embodiment of this aspect includes one or more first genetic alterations that increase activity of the PDRP protein, as compared to the wild type plant or plant part grown under the same conditions, and further includes one or more second genetic alterations that increase activity of the Rca protein, as compared to the wild type plant or plant part grown under the same conditions.

Yet another embodiment of any of the preceding aspects, which may be combined with any of the preceding embodiments, further includes one or more third genetic alterations that increase a speed of stomatal opening and closing, as compared to a wild type plant or plant part grown under the same conditions. A further embodiment of any of the preceding aspects, which may be combined with any of the preceding embodiments, further includes one or more fourth genetic alterations that increase a number of stomatal complexes and one or more fifth genetic alterations that decrease a size of stomatal complexes, as compared to a wild type plant or plant part grown under the same conditions. In an additional embodiment of any of the preceding aspects, which may be combined with any of the preceding embodiments, the one or more first genetic alterations, one or more second genetic alterations, one or more third genetic alterations, one or more fourth genetic alterations, and one or more fifth genetic alterations that increase activity include overexpression. In still another embodiment of any of the preceding aspects, the overexpression is due to a transgene overexpressing a protein with the activity being increased and/or the overexpression is due to genetic alterations in a promoter of an endogenous gene for the protein with the activity being increased. In a further embodiment of any of the preceding aspects, which may be combined with any of the preceding embodiments, the growth conditions include non-steady light, optionally field conditions or fluctuating light. In yet another embodiment of any of the preceding aspects, which may be combined with any of the preceding embodiments, the genetically altered plant or plant part has increased photosynthetic efficiency, yield, and/or water use efficiency as compared to a wild type plant or plant part grown under the same conditions. A further embodiment of any of the preceding aspects, which may be combined with any of the preceding embodiments, includes the plant being Zea mays, Saccharum officinarum, or Sorghum bicolor. Still another embodiment of any of the preceding aspects, which may be combined with any of the preceding embodiments, further includes one or more sixth genetic alterations that increase activity of PEPC, as compared to a wild type plant or plant part grown under the same conditions.

An additional aspect of this disclosure includes methods of producing the genetically altered plant or plant part of any of the preceding embodiments, including: (a) introducing the one or more first genetic alterations that increase activity of the PDRP protein, the one or more second genetic alterations that increase activity of the Rca protein and/or the Rubisco protein, or both the one or more first genetic alterations that increase activity of the PDRP protein and the one or more second genetic alterations that increase activity of the Rca protein and/or the Rubisco protein into a plant cell, tissue, or other explant of a C4 plant; (b) regenerating the plant cell, tissue, or other explant into a genetically altered C4 plantlet; and (c) growing the genetically altered C4 plantlet into a genetically altered C4 plant with the one or more genetic alterations that increase activity of the PDRP protein, the one or more genetic alterations that increase activity of the Rca protein and/or the Rubisco protein, or both the one or more genetic alterations that increase activity of the PDRP protein and the one or more genetic alterations that increase activity of the Rca protein and/or the Rubisco protein. In a further embodiment of this aspect, introducing the one or more genetic alterations that increase activity of the PDRP protein includes transforming a plant cell, tissue, or other explant of a C4 plant with a first vector including a first nucleic acid sequence encoding the PDRP protein operably linked to a second nucleic acid sequence encoding a first promoter, and/or wherein introducing the one or more genetic alterations that increase activity of the Rca protein and/or the Rubisco protein includes transforming a plant cell, tissue, or other explant of a C4 plant with a second vector including a second nucleic acid sequence encoding the Rca protein operably linked to a second promoter and/or a third vector including a third nucleic acid sequence encoding the Rubisco protein operably linked to a third promoter, optionally wherein the first vector, the second vector, and/or the third vector are introduced as a single nucleic acid construct or the first vector, the second vector, and/or the third vector are introduced separately, optionally wherein the separate introduction is into different C4 plants or C4 plant parts and the first vector, the second vector, and/or the third vector are combined through crossing the different C4 plants. In yet another embodiment of this aspect, the first promoter, the second promoter, and the third promoter, are selected from the group of a constitutive promoter, an inducible promoter, a tissue or cell type specific promoter, and an inducible, tissue or cell type specific promoter. In still another embodiment of this aspect, which may be combined with any of the preceding embodiments, introducing the one or more genetic alterations that increase activity of the PDRP protein includes transforming a plant cell, tissue, or other explant of a C4 plant with one or more first gene editing components that target a nuclear genome sequence operably linked to an endogenous PDRP protein, and/or wherein introducing the one or more genetic alterations that increase activity of the Rca protein and the Rubisco protein includes transforming a plant cell, tissue, or other explant of a C4 plant with one or more second gene editing components that target a nuclear genome sequence operably linked to an endogenous Rca protein and one or more third gene editing components that target a nuclear genome sequence operably linked to an endogenous Rubisco protein. In an additional embodiment of this aspect, the one or more first gene editing components, the one or more second gene editing components, and the one or more third gene editing components, include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. Yet another embodiment of this aspect, which may be combined with any of the preceding embodiments, further includes introducing one or more third genetic alterations that increase a speed of stomatal opening and closing, as compared to a wild type plant or plant part grown under the same conditions; introducing one or more fourth genetic alterations that increase a number of stomatal complexes and one or more fifth genetic alterations that decrease a size of stomatal complexes, as compared to a wild type plant or plant part grown under the same conditions; and/or introducing one or more sixth genetic alterations that increase activity of a PEPC protein, as compared to a wild type plant or plant part grown under the same conditions. A further embodiment of this aspect includes the plant being Zea mays, Saccharum oficinarum, or Sorghum bicolor.

An additional aspect of this disclosure includes a genetically altered plant produced by the method of any of the preceding embodiments, wherein the genetically altered plant has increased photosynthetic efficiency, increased yield potential, and/or increased water use efficiency as compared to a wild type plant or plant part grown under the same conditions.

A further aspect of this disclosure includes methods of cultivating a genetically altered plant with increased photosynthetic efficiency, including the steps of: (a) providing the genetically altered plant, wherein the plant or a part thereof includes one or more genetic alterations, and wherein the plant is a C4 plant; and (b) cultivating the genetically altered plant under conditions wherein the one or more genetic alterations increase activity of a PPDK regulatory protein (PDRP), as compared to a wild type plant grown under the same conditions, increase activity of a Rubisco activase (Rca) protein and/or a Rubisco protein, as compared to a wild type plant grown under the same conditions, or increase activity of a PDRP protein and a Rca protein and/or a Rubisco protein, as compared to a wild type plant grown under the same conditions, and wherein the increased activity of the PDRP protein, the Rca protein, and/or the Rubisco protein increases photosynthetic efficiency in the genetically altered plant as compared to the wild type plant grown under the same conditions. In an additional embodiment of this aspect, the conditions include non-steady light, optionally field conditions or fluctuating light. In yet another embodiment of this aspect, which may be combined with any of the preceding embodiments, the genetically altered plant further includes increased yield as compared to the wild type plant grown under the same conditions.

An additional aspect of this disclosure includes an isolated DNA molecule including the first vector, the second vector, and/or the third vector of any of the preceding embodiments that has a first vector, a second vector and/or a third vector; the one or more first gene editing components, the one or more second gene editing components, or the one or more third gene editing components of any of the preceding embodiments that has first gene editing components, second gene editing components, or third gene editing components; or the vector of any of the preceding embodiments that has first gene editing components, second gene editing components.

### ENUMERATED EMBODIMENTS

1. A genetically altered plant or plant part comprising one or more first genetic alterations that increase activity of a PPDK regulatory protein (PDRP), as compared to a wild type plant or plant part grown under the same conditions, wherein the genetically altered plant is a C4 plant.

2. A genetically altered plant or plant part comprising one or more first genetic alterations that increase activity of a Rubisco activase (Rca) protein and/or a Rubisco protein, as compared to a wild type plant or plant part grown under the same conditions, wherein the genetically altered plant is a C4 plant.

3. The genetically altered plant or plant part of embodiment 1, further comprising one or more second genetic alterations that increase activity of a Rubisco activase (Rca) protein and/or a Rubisco protein, as compared to a wild type plant or plant part grown under the same conditions.

4. The genetically altered plant or plant part of embodiment 3, comprising one or more first genetic alterations that increase activity of the PDRP protein, as compared to the wild type plant or plant part grown under the same conditions, and further comprising one or more second genetic alterations that increase activity of the Rca protein, as compared to the wild type plant or plant part grown under the same conditions.

5. The genetically altered plant or plant part of any one of embodiments 1-4, wherein the wherein the PDRP protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 1, SEQ ID NO: 2, SEQ ID NO: 3, SEQ ID NO: 14, SEQ ID NO: 15, SEQ ID NO: 16, or SEQ ID NO: 17; and/or the Rca protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 4, SEQ ID NO: 5, SEQ ID NO: 6, or SEQ ID NO: 7; and/or the Rubisco protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 8, SEQ ID NO: 9, SEQ ID NO: 10, SEQ ID NO: 11, SEQ ID NO: 12, or SEQ ID NO: 13.

6. The genetically altered plant or plant part of any one of embodiments 1-5, further comprising one or more third genetic alterations that increase a speed of stomatal opening and closing, as compared to a wild type plant or plant part grown under the same conditions.

7. The genetically altered plant or plant part of any one of embodiments 1-6, further comprising one or more fourth genetic alterations that increase a number of stomatal complexes and one or more fifth genetic alterations that decrease a size of stomatal complexes, as compared to a wild type plant or plant part grown under the same conditions.

8. The genetically altered plant or plant part of any one of embodiments 1-7, wherein the one or more first genetic alterations, one or more second genetic alterations, one or more third genetic alterations, one or more fourth genetic alterations, and one or more fifth genetic alterations that increase activity comprise overexpression.

9. The genetically altered plant or plant part of embodiment 8, wherein the overexpression is due to a transgene overexpressing a protein with the activity being increased and/or the overexpression is due to genetic alterations in a promoter of an endogenous gene for the protein with the activity being increased.

10. The genetically altered plant or plant part of any one of embodiments 1-9, wherein the growth conditions comprise non-steady light, optionally field conditions or fluctuating light.

11. The genetically altered plant or plant part of any one of embodiments 1-10, wherein the genetically altered plant or plant part has increased photosynthetic efficiency, yield, and/or water use efficiency as compared to a wild type plant or plant part grown under the same conditions.

12. The genetically altered plant or plant part of any one of embodiments 1-11, wherein the plant is Zea mays, Saccharum officinarum, or Sorghum bicolor.

13. The genetically altered plant or plant part of any one of embodiments 1-12, further comprising one or more sixth genetic alterations that increase activity of PEPC, as compared to a wild type plant or plant part grown under the same conditions.

14. A method of producing the genetically altered plant or plant part of any one of embodiments 1-13, comprising:
a) introducing the one or more first genetic alterations that increase activity of the PDRP protein, the one or more second genetic alterations that increase activity of the Rca protein and/or the Rubisco protein, or both the one or more first genetic alterations that increase activity of the PDRP protein and the one or more second genetic alterations that increase activity of the Rca protein and/or the Rubisco protein into a plant cell, tissue, or other explant of a C4 plant;
b) regenerating the plant cell, tissue, or other explant into a genetically altered C4 plantlet; and
c) growing the genetically altered C4 plantlet into a genetically altered C4 plant with the one or more genetic alterations that increase activity of the PDRP protein, the one or more genetic alterations that increase activity of the Rca protein and/or the Rubisco protein, or both the one or more genetic alterations that increase activity of the PDRP protein and the one or more genetic alterations that increase activity of the Rca protein and/or the Rubisco protein.

15. The method of embodiment 14, wherein introducing the one or more genetic alterations that increase activity of the PDRP protein comprises transforming a plant cell, tissue, or other explant of a C4 plant with a first vector comprising a first nucleic acid sequence encoding the PDRP protein operably linked to a second nucleic acid sequence encoding a first promoter, and/or wherein introducing the one or more genetic alterations that increase activity of the Rca protein and/or the Rubisco protein comprises transforming a plant cell, tissue, or other explant of a C4 plant with a second vector comprising a second nucleic acid sequence encoding the Rca protein operably linked to a second promoter and/or a third vector comprising a third nucleic acid sequence encoding the Rubisco protein operably linked to a third promoter, optionally wherein the first vector, the second vector, and/or the third vector are introduced as a single nucleic acid construct or the first vector, the second vector, and/or the third vector are introduced separately, optionally wherein the separate introduction is into different C4 plants or C4 plant parts and the first vector, the second vector, and/or the third vector are combined through crossing the different C4 plants.

16. The method of embodiment 15, wherein the first promoter, the second promoter, and the third promoter, are selected from the group of a constitutive promoter, an inducible promoter, a tissue or cell type specific promoter, and an inducible, tissue or cell type specific promoter.

17. The method of any one of embodiments 14-16, wherein introducing the one or more genetic alterations that increase activity of the PDRP protein comprises transforming a plant cell, tissue, or other explant of a C4 plant with one or more first gene editing components that target a nuclear genome sequence operably linked to an endogenous PDRP protein, and/or wherein introducing the one or more genetic alterations that increase activity of the Rca protein and the Rubisco protein comprises transforming a plant cell, tissue, or other explant of a C4 plant with one or more second gene editing components that target a nuclear genome sequence operably linked to an endogenous Rca protein and one or more third gene editing components that target a nuclear genome sequence operably linked to an endogenous Rubisco protein.

18. The method of embodiment 17, wherein the one or more first gene editing components, the one or more second gene editing components, and the one or more third gene editing components, comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

19. The method of any one of embodiments 14-18, further comprising introducing one or more third genetic alterations that increase a speed of stomatal opening and closing, as compared to a wild type plant or plant part grown under the same conditions; introducing one or more fourth genetic alterations that increase a number of stomatal complexes and one or more fifth genetic alterations that decrease a size of stomatal complexes, as compared to a wild type plant or plant part grown under the same conditions; and/or introducing one or more sixth genetic alterations that increase activity of a PEPC protein, as compared to a wild type plant or plant part grown under the same conditions.

20. The method of any one of embodiments 14-19, wherein the plant is Zea mays, Saccharum oficinarum, or Sorghum bicolor.

21. A genetically altered plant produced by the method of any one of embodiments 14-20, wherein the genetically altered plant has increased photosynthetic efficiency, increased yield potential, and/or increased water use efficiency as compared to a wild type plant or plant part grown under the same conditions.

22. A method of cultivating a genetically altered plant with increased photosynthetic efficiency, comprising the steps of:
a) providing the genetically altered plant, wherein the plant or a part thereof comprises one or more genetic alterations, and wherein the plant is a C4 plant; and
b) cultivating the genetically altered plant under conditions wherein the one or more genetic alterations increase activity of a PPDK regulatory protein (PDRP), as compared to a wild type plant grown under the same conditions, increase activity of a Rubisco activase (Rca) protein and/or a Rubisco protein, as compared to a wild type plant grown under the same conditions, or increase activity of a PDRP protein and a Rca protein and/or a Rubisco protein, as compared to a wild type plant grown under the same conditions, and wherein the increased activity of the PDRP protein, the Rca protein, and/or the Rubisco protein increases photosynthetic efficiency in the genetically altered plant as compared to the wild type plant grown under the same conditions.

23. The method of embodiment 22, wherein the PDRP protein comprises the amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% Yo sequence identity, or 100% sequence identity to one of SEQ ID NO: 1, SEQ ID NO; 2, SEQ ID NO; 3, SEQ ID NO; 14, SEQ ID NO: 15, SEQ ID NO: 16, or SEQ ID NO: 17; and/or the Rca protein comprises the amino acid sequence having at least 70% sequence identity, at least 75% sequence identity, at least 80% sequence identity, at least 85% sequence identity, at least 900% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 4, SEQ ID NO: 5, SEQ ID NO: 6, or SEQ ID NO: 7; and/or the Rubisco protein comprises the amino acid sequence having at least⁷⁰⁰/sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99%/6 sequence identity, or 100% sequence identity to one of SEQ ID NO: 8, SEQ ID NO: 9, SEQ ID NO: 10, SEQ ID NO: 11, SEQ ID NO: 12, or SEQ ID NO: 13.

24. The method of embodiment 22 or embodiment 23, wherein the conditions comprise non-steady light, optionally field conditions or fluctuating light.

25. The method of any one of embodiments 22-24, wherein the genetically altered plant further comprises increased yield as compared to the wild type plant grown under the same conditions.

26. An isolated DNA molecule comprising the first vector, the second vector, and/or the third vector of embodiment 15 or embodiment 16; the one or more first gene editing components, the one or more second gene editing components, or the one or more third gene editing components of embodiment 17 or embodiment 18; or the vector of embodiment 18.

27. A genetically altered plant or plant part comprising one or more first genetic alterations that increase activity of a PPDK regulatory protein (PDRP) as compared to a wild type plant or plant part grown under the same conditions, wherein the genetically altered plant is a C4 plant, and optionally further comprising one or more second genetic alterations that increase activity of a Rubisco activase (Rca) protein and/or a Rubisco protein, as compared to a wild type plant or plant part grown under the same conditions.

28. A genetically altered plant or plant part comprising one or more first genetic alterations that increase activity of a Rubisco activase (Rca) protein and/or a Rubisco protein as compared to a wild type plant or plant part grown under the same conditions, wherein the genetically altered plant is a C4 plant, and optionally further comprising one or more first genetic alterations that increase activity of the PDRP protein, as compared to the wild type plant or plant part grown under the same conditions, and further comprising one or more second genetic alterations that increase activity of the Rca protein, as compared to the wild type plant or plant part grown under the same conditions.

29. The genetically altered plant or plant part of embodiment 27 or embodiment 28, wherein the PDRP protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 1, SEQ ID NO: 2, SEQ ID NO: 3, SEQ ID NO: 14, SEQ ID NO: 15, SEQ ID NO: 16, or SEQ ID NO: 17; and/or the Rca protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 4, SEQ ID NO: 5, SEQ ID NO: 6, or SEQ ID NO: 7; and/or the Rubisco protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 8, SEQ ID NO: 9, SEQ ID NO: 10, SEQ ID NO: 11, SEQ ID NO: 12, or SEQ ID NO: 13.

30. The genetically altered plant or plant part of any one of embodiments 27-29, further comprising one or more third genetic alterations that increase a speed of stomatal opening and closing, as compared to a wild type plant or plant part grown under the same conditions.

31. The genetically altered plant or plant part of any one of embodiments 27-30, further comprising one or more fourth genetic alterations that increase a number of stomatal complexes and one or more fifth genetic alterations that decrease a size of stomatal complexes, as compared to a wild type plant or plant part grown under the same conditions.

32. The genetically altered plant or plant part of any one of embodiments 27-31, wherein the one or more first genetic alterations, one or more second genetic alterations, one or more third genetic alterations, one or more fourth genetic alterations, and one or more fifth genetic alterations that increase activity comprise overexpression, and wherein the overexpression is due to a transgene overexpressing a protein with the activity being increased and/or the overexpression is due to genetic alterations in a promoter of an endogenous gene for the protein with the activity being increased.

33. The genetically altered plant or plant part of any one of embodiments 27-32, wherein the growth conditions comprise non-steady light, optionally field conditions or fluctuating light, and wherein the genetically altered plant or plant part has increased photosynthetic efficiency, yield, and/or water use efficiency as compared to a wild type plant or plant part grown under the same conditions.

34. The genetically altered plant or plant part of any one of embodiments 27-33, wherein the plant is Zea mays, Saccharum officinarum, or Sorghum bicolor.

35. The genetically altered plant or plant part of any one of embodiments 27-34, further comprising one or more sixth genetic alterations that increase activity of PEPC, as compared to a wild type plant or plant part grown under the same conditions.

36. A method of producing the genetically altered plant or plant part of any one of embodiments 27-35, comprising:
a) introducing the one or more first genetic alterations that increase activity of the PDRP protein, the one or more second genetic alterations that increase activity of the Rca protein and/or the Rubisco protein, or both the one or more first genetic alterations that increase activity of the PDRP protein and the one or more second genetic alterations that increase activity of the Rca protein and/or the Rubisco protein into a plant cell, tissue, or other explant of a C4 plant;
b) regenerating the plant cell, tissue, or other explant into a genetically altered C4 plantlet; and
c) growing the genetically altered C4 plantlet into a genetically altered C4 plant with the one or more genetic alterations that increase activity of the PDRP protein, the one or more genetic alterations that increase activity of the Rca protein and/or the Rubisco protein, or both the one or more genetic alterations that increase activity of the PDRP protein and the one or more genetic alterations that increase activity of the Rca protein and/or the Rubisco protein.

37. The method of embodiment 36, wherein introducing the one or more genetic alterations that increase activity of the PDRP protein comprises transforming a plant cell, tissue, or other explant of a C4 plant with a first vector comprising a first nucleic acid sequence encoding the PDRP protein operably linked to a second nucleic acid sequence encoding a first promoter, and/or wherein introducing the one or more genetic alterations that increase activity of the Rca protein and/or the Rubisco protein comprises transforming a plant cell, tissue, or other explant of a C4 plant with a second vector comprising a second nucleic acid sequence encoding the Rca protein operably linked to a second promoter and/or a third vector comprising a third nucleic acid sequence encoding the Rubisco protein operably linked to a third promoter, optionally wherein the first vector, the second vector, and/or the third vector are introduced as a single nucleic acid construct or the first vector, the second vector, and/or the third vector are introduced separately, optionally wherein the separate introduction is into different C4 plants or C4 plant parts and the first vector, the second vector, and/or the third vector are combined through crossing the different C4 plants.

38. The method of embodiment 37, wherein the first promoter, the second promoter, and the third promoter, are selected from the group of a constitutive promoter, an inducible promoter, a tissue or cell type specific promoter, and an inducible, tissue or cell type specific promoter.

39. The method of any one of embodiments 36-38, wherein introducing the one or more genetic alterations that increase activity of the PDRP protein comprises transforming a plant cell, tissue, or other explant of a C4 plant with one or more first gene editing components that target a nuclear genome sequence operably linked to an endogenous PDRP protein, and/or wherein introducing the one or more genetic alterations that increase activity of the Rca protein and the Rubisco protein comprises transforming a plant cell, tissue, or other explant of a C4 plant with one or more second gene editing components that target a nuclear genome sequence operably linked to an endogenous Rca protein and one or more third gene editing components that target a nuclear genome sequence operably linked to an endogenous Rubisco protein.

40. The method of embodiment 39, wherein the one or more first gene editing components, the one or more second gene editing components, and the one or more third gene editing components, comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

41. The method of any one of embodiments 36-40, further comprising introducing one or more third genetic alterations that increase a speed of stomatal opening and closing, as compared to a wild type plant or plant part grown under the same conditions; introducing one or more fourth genetic alterations that increase a number of stomatal complexes and one or more fifth genetic alterations that decrease a size of stomatal complexes, as compared to a wild type plant or plant part grown under the same conditions; and/or introducing one or more sixth genetic alterations that increase activity of a PEPC protein, as compared to a wild type plant or plant part grown under the same conditions.

42. The method of any one of embodiments 36-41, wherein the plant is Zea mays, Saccharum oqficinarum, or Sorghum bicolor.

43. A genetically altered plant produced by the method of any one of embodiments 36-42, wherein the genetically altered plant has increased photosynthetic efficiency, increased yield potential, and/or increased water use efficiency as compared to a wild type plant or plant part grown under the same conditions.

44. A method of cultivating a genetically altered plant with increased photosynthetic efficiency, comprising the steps of:
a) providing the genetically altered plant, wherein the plant or a part thereof comprises one or more genetic alterations, and wherein the plant is a C4 plant; and
b) cultivating the genetically altered plant under conditions wherein the one or more genetic alterations increase activity of a PPDK regulatory protein (PDRP), as compared to a wild type plant grown under the same conditions, increase activity of a Rubisco activase (Rca) protein and/or a Rubisco protein comprising, as compared to a wild type plant grown under the same conditions, or increase activity of a PDRP protein and a Rca protein and/or a Rubisco protein, as compared to a wild type plant grown under the same conditions, and wherein the increased activity of the PDRP protein, the Rca protein, and/or the Rubisco protein increases photosynthetic efficiency in the genetically altered plant as compared to the wild type plant grown under the same conditions.

45. The method of embodiment 44, wherein the PDRP protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99⁰/% sequence identity, or 100/0 sequence identity to one of SEQ ID NO: 1, SEQ ID NO: 2, SEQ ID NO: 3, SEQ ID NO: 14, SEQ ID NO: 15, SEQ ID NO: 16, or SEQ ID NO: 17; and/or the Rca protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 4, SEQ ID NO: 5, SEQ ID NO: 6, or SEQ ID NO: 7; and/or the Rubisco protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 8, SEQ ID NO: 9, SEQ ID NO: 10, SEQ ID NO: 11, SEQ ID NO: 12, or SEQ ID NO: 13.

46. The method of embodiment 44 or embodiment 45, wherein the conditions comprise non-steady light, optionally field conditions or fluctuating light.

47. The method of any one of embodiments 44-46, wherein the genetically altered plant further comprises increased yield as compared to the wild type plant grown under the same conditions.

48. An isolated DNA molecule comprising the first vector, the second vector, and/or the third vector of embodiment 37; the one or more first gene editing components, the one or more second gene editing components, or the one or more third gene editing components of embodiment 39; or the vector of embodiment 40.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a metabolic model schematic of C4 photosynthesis. The model includes all metabolites and enzymes of photosynthetic carbon metabolism as detailed previously (Wang, Y. et al. (2014) Three distinct biochemical subtypes of C4 photosynthesis? A modelling analysis. Journal of Experimental Botany, 65, 3567-3578; Wang, Y. et al. (2014b) Elements Required for an Efficient NADP-Malic Enzyme Type C4 Photosynthesis. Plant Physiology, 164, 2231-2246). Here only the enzymes that are light modulated and therefore modified in this new dynamic model are indicated. Rectangles are driving environmental variables affecting enzyme activities (shaded ovals in boxes) and stomatal conductance. Blocks are state variables calculated from leaf energy balance for T_{leaf}, dynamic stomatal response model for stomatal conductance (gₛ), and from the external [CO₂], gₛ and predicted leaf CO₂ uptake rate for Cᵢ.

FIGS. 2A-2B show simulated induction of leaf CO₂ uptake (A) and bundle-sheath leakiness (ϕ) with various dynamic regulating settings. FIG. 2A shows simulated induction of leaf CO₂ uptake (A). FIG. 2B shows simulated induction of bundle-sheath leakiness (ϕ). In FIGS. 2A-2B, scenario (1) uses the original metabolic model (Wang, Y. et al. (2014) Three distinct biochemical subtypes of C4 photosynthesis? A modelling analysis. Journal of Experimental Botany, 65, 3567-3578; Wang, Y. et al. (2014b) Elements Required for an Efficient NADP-Malic Enzyme Type C4 Photosynthesis. Plant Physiology, 164, 2231-2246), which assumes steady-state enzyme activity and stomatal conductance from time zero, i.e. throughout. Scenarios (2) DyPPDK and (3) DyRubisco add to the steady-state model (1) the induction responses of PPDK and Rubisco, respectively, regulated by the action of the PPDK regulatory protein (PDRP) and by the action of Rubisco activase (Rca) for Rubisco. Scenario (4) combines these two and (5) includes all light regulated enzymes. Scenario (6) superimposes stomatal opening on scenario (5). The induction simulates transfer from darkness to full sunlight, 1800 μmol m⁻² s⁻¹. The input parameters are those of Table 2, column “Original values”.

FIGS. 3A-3C show simulated dynamic leaf CO₂ uptake (A) from dark to high light with variation in PPDK regulatory protein (PDRP), τ_{Rubisco}, and stomata opening speed (g_{s_ki}). FIG. 3A shows simulated dynamic leaf CO₂ uptake (A) from dark to high light with variation in PPDK regulatory protein (PDRP). FIG. 3B shows simulated dynamic leaf CO₂ uptake (A) from dark to high light with variation in τ_{Rubisco}, FIG. 3C shows simulated dynamic leaf CO₂ uptake (A) from dark to high light with variation in stomata opening speed (g_{s_ki}). In FIGS. 3A-3C, τ_{Rubisco} is the time constant of Rubisco activation reaction catalyzed by Rubisco activase; [PDRP] is the concentration of PPDK regulatory protein; kᵢ is the rate constant of stomata conductance increase. After dark adaptation, light intensity was set as 1800 μmol m⁻² s⁻¹. The input parameters are those of Table 2, column “Original values”.

FIGS. 4A-4E show measured gas exchange parameters in dark to high light (1800 μmol m⁻² s⁻¹) transition. FIG. 4A shows leaf CO₂ uptake rate (A). FIG. 4B shows intercellular CO₂ concentration (Cᵢ). FIG. 4C shows stomata conductance (gₛ). FIG. 4D shows non photochemical quenching (NPQ). FIG. 4E shows intrinsic water use efficiency (iWUE). In FIGS. 4A-4E, bars represent standard error of the mean for six plants.

FIGS. 5A-5F show simulated dynamic photosynthesis (A) and stomata conductance (gₛ) under fluctuating light. The simulation uses the parameters of non-steady-state photosynthesis of scenario 6 in FIGS. 2A-2B, but calibrated to the measured steady-state photosynthesis of FIGS. 4A-4E. FIG. 5A shows leaf CO₂ uptake (A) of Zea mays (maize) B73. FIG. 5B shows stomata conductance (gₛ) of Zea mays (maize) B73. FIG. 5C shows leaf CO₂ uptake (A) of Sorghum bicolor (sorghum) Tx430. FIG. 5D shows stomata conductance (gₛ) of Sorghum bicolor (sorghum) Tx430. FIG. 5E shows leaf CO₂ uptake (A) of Saccharum officinarum (sugarcane) CP88-1762. FIG. 5F stomata conductance (gₛ) of Saccharum officinarum (sugarcane) CP88-1762. In FIGS. 5A-5F, leaf CO₂ uptake (A) of the youngest fully expanded leaf was measured on 30 day-old maize B73, sugarcane CP88-1762 and sorghum Tx430 plants with a gas exchange system (LI-6800; LI-COR, Lincoln, NE, USA). The measurements were made on six replicate plants. Lines are the simulated results, and dots are measured data. Leaves were first dark adapted for 30 min (not shown). After dark adaptation, the leaves undergo three light change steps, light intensity was set as 1800 μmol m⁻² s⁻¹, 200 μmol m⁻²s⁻¹ and 1800 μmol m⁻²s⁻¹ for each 1800 s step. The input parameters are those of Table 2, columns “Maize, Sorghum and Sugarcane” respectively.

FIGS. 6A-6C show simulated changes of sensitivity coefficients of key parameters through photosynthetic induction. FIG. 6A shows simulated changes for Zea mays (maize) B73. FIG. 6B shows simulated changes for Sorghum bicolor (sorghum) Tx430. FIG. 6C shows simulated changes for Saccharum officinarum (sugarcane) CP88-1762. In FIGS. 6A-6C, After dark adaptation, PAR was set as 1800 μmol m⁻²s⁻¹. To determine which steps in the system, exert the strongest control on dynamic photosynthesis rate, a sensitivity analysis was performed by varying each parameter+/−1%. Sensitivity coefficients are calculated as the ratio of change in the value of the parameter divided by change in leaf CO₂ uptake rate (A), individually. If a 1% change in parameter x results in a 1% change in A the sensitivity coefficient is 1; while if the change in A is zero, then the sensitivity coefficient is 0, meaning that no effect is exerted by that parameter. k_{i_gs} is the time constant of stomata opening; τ_{Rubisco} is the time constant of Rubisco activation; [PDRP] is the concentration of PPDK regulatory protein.

FIGS. 7A-7F show the control coefficient of the maximum activity of photosynthetic enzymes (Vmax) during induction. FIG. 7A shows the predicted control coefficients of C4 cycle enzymes for Zea mays (maize) B73, with ME shown at the top, PPDK shown second from the top, PEPC shown in the middle, MDH shown second from the bottom, and Mutase & Enolase shown at the bottom at time 1200. FIG. 7B shows the predicted control coefficients of Calvin-Benson cycle enzymes for Zea mays (maize) B73, with Rubisco shown at the top, SBPase shown second from the top, PRK shown in the middle, DAPDH shown second from the bottom, and FBPase shown at the bottom at time 300. FIG. 7C shows the predicted control coefficients of C4 cycle enzymes for Sorghum bicolor (sorghum) Tx430, with ME shown at the top, PPDK shown second from the top, PEPC shown in the middle, Mutase & Enolase shown second from the bottom, and MDH shown at the bottom at time 1200. FIG. 7D shows the predicted control coefficients of Calvin-Benson cycle enzymes for Sorghum bicolor (sorghum) Tx430, with Rubisco shown at the top, SBPase shown second from the top, PRK shown in the middle, DAPDH shown second from the bottom, and FBPase shown at the bottom between time 0 and 300. FIG. 7E shows the predicted control coefficients of C4 cycle enzymes for Saccharum oficinarum (sugarcane) CP88-1762, with PEPC shown at the top, ME shown second from the top, MDH shown in the middle (overlapping with ME), Mutase & Enolase shown second from the bottom, and PPDK shown at the bottom at time 1200. FIG. 7F shows the predicted control coefficients of Calvin-Benson cycle enzymes for Saccharum officinarum (sugarcane) CP88-1762, with Rubisco shown at the top, SBPase shown second from the top, PRK shown in the middle, DAPDH shown second from the bottom, and FBPase shown at the bottom at time 300. In FIGS. 7A-7F, after dark adaptation, light intensity was set as 1800 μmol m⁻²s⁻¹. The photosynthetic enzymes shown include: PEPC, phosphoenolpyruvate carboxylase; PPDK, pyruvate, phosphate dikinase; MDH, malate dehydrogenase (NADP+); ME, NADP-malic enzyme; Mutase and Enolase; Rubisco, ribulose-bisphosphate carboxylase; GAPDH, glyceraldehyde-3-phosphate dehydrogenase (NADP+); SBPase, sedoheptulose-bisphosphatase; FBPase, fructose-bisphosphatase; PRK, Phosphoribulokinase.

FIG. 8 shows simulated CO₂ leakiness (p) dynamics for Zea mays (maize) B73, Sorghum bicolor (sorghum) Tx430, and Saccharum oficinarum (sugarcane) CP88-1762 during photosynthetic induction following 30 minutes of dark adaptation. Light was set as 1800 μmol m² s⁻¹. The input parameters are those of Table 2, columns “Maize, Sorghum and Sugarcane” respectively.

FIGS. 9A-9D show simulated photosynthetic induction using metabolic model without posttranslational regulation of enzymes and delay of stomata conductance. FIG. 9A shows net photosynthesis rate. FIG. 9B shows leakiness. FIG. 9C shows relative concentrations of C4 cycle metabolites, with OAA shown at the top, PEP shown second from the top, PYR shown third from the top, and MAL shown at the bottom between 300 and 600 seconds. FIG. 9D shows relative concentration of Calvin-Benson cycle metabolites, with SBP shown at the top, PGA shown second from the top, T3P shown in the middle, FBP shown second from the bottom, and HexP shown at the bottom at 600 seconds.

FIGS. 10A-10B show estimated influence of mutase and enolase on photosynthetic induction using metabolic model without posttranslational regulation of enzymes and delay of stomata conductance. FIG. 10A shows A (pmol m⁻²s⁻¹), with the lines in the same order from top to bottom as in the figure inset (i.e., 3.0 μmol at the top and 0.33 μmol at the bottom) between 0 and 300 seconds. FIG. 10B shows leakiness, the lines in the same order from top to bottom as in the figure inset (i.e., 3.0 μmol at the top and 0.33 μmol at the bottom) at 600 seconds.

FIGS. 11A-11B show estimation of ƒ_{vPEPC} and Γ_{vRubisco} using least squares method.

FIG. 11A shows the slope of measured A-Ci curve, which was used to estimate ƒ_{vPEPC}. FIG. 11B shows the plateau of A-Cᵢ curve, which was used for ƒ_{vRubisco}.

FIG. 12 shows a semilogarithmic plot of the difference between the photosynthesis (A) and maximum photosynthesis (Ar) as a function of time. Time courses for photosynthesis were measured following a change in PPFD from 0 to 1800 μmol m² s⁻¹. Data between 3-7 min of the measured curves was used to estimate the τ_{Rubisco} (Table 2)

FIG. 13 shows estimation of PPDK regulatory protein concentration ([PDRP]) using measured photosynthetic induction curves. PDRP concentration was estimated using least squares method, minimized the sum of square of the difference between dynamic model estimated and measured CO₂ uptake rate in the beginning of the photosynthetic induction (1-3 min).

FIGS. 14A-14B show measured CO₂ response curves and light response curves of Zea mays (maize) B73, SOrghum bicolor (sorghum) Tx430, and Saccharum officinarum (sugarcane) CP88-1762. FIG. 14A shows measured CO₂ response curves, with Sorghum Tx430 as the top line, Maize B73 as the middle line, and Sugarcane CP88-1762 as the bottom line, all following the same trend. FIG. 14B shows measured light response curves, with Sorghum Tx430 as the top line, Maize B73 as the middle line, and Sugarcane CP88-1762 as the bottom line, all following the same trend. In FIGS. 14A-14B, error bars represent standard errors, six replicates were measured for each species.

FIGS. 15A-15C show calculated Ball-Berry slope and intercept using gas exchange data from light response curves. FIG. 15A shows the calculated Ball-Berry slope and intercept using gas exchange data from light response curves of Zea mays (maize) B73. For P1, the trend line was y=0.042+0.0936; for P2, the trend line was y=0.0425x+0.0258; for P3, the trend line was y=0.0537x−0.0085; for P4, the trend line was y=0.0459x+0.007; and for P5, the trend line was y=0.0663x+0.171. FIG. 15B shows the calculated Ball-Berry slope and intercept using gas exchange data from light response curves of Sorghum bicolor (sorghum) Tx430. For P1, the trend line was y=0.0447x+0.0531; for P2, the trend line was y=0.0469x+0.053; for P3, the trend line was y=0.0567x−0.0128; for P4, the trend line was y=0.0456x-0.0125; for P5, the trend line was y=0.0476x−0.0069; and for P6, the trend line was y=0.0539x−0.0035. FIG. 15C shows the calculated Ball-Berry slope and intercept using gas exchange data from light response curves of Saccharum oficinarum (sugarcane) CP88-1762. For P1, the trend line was y=0.044x+0.0224; for P2, the trend line was y=0.0473x+0.0485; for P3, the trend line was y=0.0515x+0.0152; for P4, the trend line was y=0.0492x+0.0106; for P5, the trend line was y=0.0542x+0.0376; and for P6, the trend line was y=0.0503x+0.0316. In FIGS. 15A-15C, different shapes and shading represent each individual measurement.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Genetically Altered Plants and Seeds

An aspect of the disclosure includes a genetically altered plant or plant part including one or more first genetic alterations that increase activity of a PPDK regulatory protein (PDRP), as compared to a wild type plant or plant part grown under the same conditions, wherein the genetically altered plant is a C4 plant. The wild type plant is also a C4 plant.

A further aspect of the disclosure includes a genetically altered plant or plant part including one or more first genetic alterations that increase activity of a Rubisco activase (Rca) protein and/or a Rubisco protein, as compared to a wild type plant or plant part grown under the same conditions, wherein the genetically altered plant is a C4 plant. The wild type plant is also a C4 plant.

An additional embodiment of this aspect, which may be combined with any of the preceding embodiments that has one or more first genetic alterations that increase activity of a PPDK regulatory protein (PDRP), further includes one or more second genetic alterations that increase activity of a Rubisco activase (Rca) protein and/or a Rubisco protein, as compared to a wild type plant or plant part grown under the same conditions. Still another embodiment of this aspect includes one or more first genetic alterations that increase activity of the PDRP protein, as compared to the wild type plant or plant part grown under the same conditions, and further includes one or more second genetic alterations that increase activity of the Rca protein, as compared to the wild type plant or plant part grown under the same conditions. Without wishing to be bound by theory, increasing activity of PDRP and Rca in concert balances the C3 and C4 cycles.

Yet another embodiment of any of the preceding aspects, which may be combined with any of the preceding embodiments, further includes one or more third genetic alterations that increase a speed of stomatal opening and closing, as compared to a wild type plant or plant part grown under the same conditions. Combined thermal and modulated fluorescence techniques may provide a potential high-throughput means to screen germplasm for this trait (Vialet-Chabrand, S. and Lawson, T. (2019) Dynamic leaf energy balance: deriving stomatal conductance from thermal imaging in a dynamic environment. Journal of experimental botany, 70, 2839-2855; Vialet-Chabrand, S. and Lawson, T. (2020) Thermography methods to assess stomatal behaviour in a dynamic environment. Journal of experimental botany, 71, 2329-2338). A further embodiment of any of the preceding aspects, which may be combined with any of the preceding embodiments, further includes one or more fourth genetic alterations that increase a number of stomatal complexes and one or more fifth genetic alterations that decrease a size of stomatal complexes, as compared to a wild type plant or plant part grown under the same conditions. In an additional embodiment of any of the preceding aspects, which may be combined with any of the preceding embodiments, the one or more first genetic alterations, one or more second genetic alterations, one or more third genetic alterations, one or more fourth genetic alterations, and one or more fifth genetic alterations that increase activity include overexpression. In still another embodiment of any of the preceding aspects, the overexpression is due to a transgene overexpressing a protein with the activity being increased and/or the overexpression is due to genetic alterations in a promoter of an endogenous gene for the protein with the activity being increased. In yet another embodiment of any of the preceding aspects, which may be combined with any of the preceding embodiments, the concentration of PDRP protein is increased, as compared to a wild type plant or plant part grown under the same conditions. In an additional embodiment of any of the preceding aspects, which may be combined with any of the preceding embodiments, the speed of Rubisco activation is increased as compared to a wild type plant or plant part grown under the same conditions. In still another embodiment of any of the preceding aspects, which may be combined with any of the preceding embodiments, both the PDRP protein concentration and the speed of Rubisco activation are increased, and optionally, the increase in the speed of Rubisco activation is larger. In a further embodiment of any of the preceding aspects, which may be combined with any of the preceding embodiments, the growth conditions include non-steady light, optionally field conditions or fluctuating light. The growth conditions may be the fluctuating light of a crop canopy. In yet another embodiment of any of the preceding aspects, which may be combined with any of the preceding embodiments, the genetically altered plant or plant part has increased photosynthetic efficiency, yield, and/or water use efficiency as compared to a wild type plant or plant part grown under the same conditions. A further embodiment of any of the preceding aspects, which may be combined with any of the preceding embodiments, includes the plant being Zea mays (e.g., maize, corn), Saccharum oficinarum (e.g., sugarcane, Saccharum spp., Saccharum hybrids), or Sorghum bicolor (e.g., sorghum). Still another embodiment of any of the preceding aspects, which may be combined with any of the preceding embodiments, further includes one or more sixth genetic alterations that increase activity of PEPC, as compared to a wild type plant or plant part grown under the same conditions.

### Methods of Producing and Cultivating Genetically Altered Plants

An additional aspect of this disclosure includes methods of producing the genetically altered plant or plant part of any of the preceding embodiments, including. (a) introducing the one or more first genetic alterations that increase activity of the PDRP protein, the one or more second genetic alterations that increase activity of the Rca protein and/or the Rubisco protein, or both the one or more first genetic alterations that increase activity of the PDRP protein and the one or more second genetic alterations that increase activity of the Rca protein and/or the Rubisco protein into a plant cell, tissue, or other explant of a C4 plant; (b) regenerating the plant cell, tissue, or other explant into a genetically altered C4 plantlet; and (c) growing the genetically altered C4 plantlet into a genetically altered C4 plant with the one or more genetic alterations that increase activity of the PDRP protein, the one or more genetic alterations that increase activity of the Rca protein and/or the Rubisco protein, or both the one or more genetic alterations that increase activity of the PDRP protein and the one or more genetic alterations that increase activity of the Rca protein and/or the Rubisco protein. In a further embodiment of this aspect, introducing the one or more genetic alterations that increase activity of the PDRP protein includes transforming a plant cell, tissue, or other explant of a C4 plant with a first vector including a first nucleic acid sequence encoding the PDRP protein operably linked to a second nucleic acid sequence encoding a first promoter, and/or wherein introducing the one or more genetic alterations that increase activity of the Rca protein and/or the Rubisco protein includes transforming a plant cell, tissue, or other explant of a C4 plant with a second vector including a second nucleic acid sequence encoding the Rca protein operably linked to a second promoter and/or a third vector including a third nucleic acid sequence encoding the Rubisco protein operably linked to a third promoter, optionally wherein the first vector, the second vector, and/or the third vector are introduced as a single nucleic acid construct or the first vector, the second vector, and/or the third vector are introduced separately, optionally wherein the separate introduction is into different C4 plants or C4 plant parts and the first vector, the second vector, and/or the third vector are combined through crossing the different C4 plants. In yet another embodiment of this aspect, the first promoter, the second promoter, and the third promoter, are selected from the group of a constitutive promoter, an inducible promoter, a tissue or cell type specific promoter, and an inducible, tissue or cell type specific promoter. In still another embodiment of this aspect, which may be combined with any of the preceding embodiments, introducing the one or more genetic alterations that increase activity of the PDRP protein includes transforming a plant cell, tissue, or other explant of a C4 plant with one or more first gene editing components that target a nuclear genome sequence operably linked to an endogenous PDRP protein, and/or wherein introducing the one or more genetic alterations that increase activity of the Rca protein and the Rubisco protein includes transforming a plant cell, tissue, or other explant of a C4 plant with one or more second gene editing components that target a nuclear genome sequence operably linked to an endogenous Rca protein and one or more third gene editing components that target a nuclear genome sequence operably linked to an endogenous Rubisco protein. In an additional embodiment of this aspect, the one or more first gene editing components, the one or more second gene editing components, and the one or more third gene editing components, include a ribonucleoprotein complex that targets the nuclear genome sequence; a vector including a TALEN protein encoding sequence, wherein the TALEN protein targets the nuclear genome sequence; a vector including a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence. In an additional embodiment of this aspect, which may be combined with any of the preceding embodiments, the genetic alterations that increase activity include overexpression. Yet another embodiment of this aspect, which may be combined with any of the preceding embodiments, further includes introducing one or more third genetic alterations that increase a speed of stomatal opening and closing, as compared to a wild type plant or plant part grown under the same conditions; introducing one or more fourth genetic alterations that increase a number of stomatal complexes and one or more fifth genetic alterations that decrease a size of stomatal complexes, as compared to a wild type plant or plant part grown under the same conditions; and/or introducing one or more sixth genetic alterations that increase activity of a PEPC protein, as compared to a wild type plant or plant part grown under the same conditions. A further embodiment of this aspect includes the plant being Zea mays (e.g., maize, corn), Saccharum officinarum (e.g., sugarcane, Saccharum spp., Saccharum hybrids), or Sorghum bicolor (e.g., sorghum). In yet another embodiment of this aspect, which may be combined with any of the preceding embodiments, the growth conditions include non-steady light, optionally field conditions or fluctuating light. The growth conditions may be the fluctuating light of a crop canopy.

An additional aspect of this disclosure includes a genetically altered plant produced by the method of any of the preceding embodiments, wherein the genetically altered plant has increased photosynthetic efficiency, increased yield potential, and/or increased water use efficiency as compared to a wild type plant or plant part grown under the same conditions. The genetically altered plant and the wild type plant are C4 plants. In still another embodiment of this aspect, which may be combined with any of the preceding embodiments, the genetically altered plant includes increased activity of the PDRP protein and increased activity of the Rca protein, as compared to a wild type plant grown under the same conditions.

A further aspect of this disclosure includes methods of cultivating a genetically altered plant with increased photosynthetic efficiency, including the steps of: (a) providing the genetically altered plant, wherein the plant or a part thereof includes one or more genetic alterations, and wherein the plant is a C4 plant; and (b) cultivating the genetically altered plant under conditions wherein the one or more genetic alterations increase activity of a PPDK regulatory protein (PDRP), as compared to a wild type plant grown under the same conditions, increase activity of a Rubisco activase (Rca) protein and/or a Rubisco protein, as compared to a wild type plant grown under the same conditions, or increase activity of a PDRP protein and a Rca protein and/or a Rubisco protein, as compared to a wild type plant grown under the same conditions, and wherein the increased activity of the PDRP protein, the Rca protein, and/or the Rubisco protein increases photosynthetic efficiency in the genetically altered plant as compared to the wild type plant grown under the same conditions. In still another embodiment of this aspect, which may be combined with any of the preceding embodiments, the genetically altered plant includes increased activity of the PDRP protein and increased activity of the Rca protein, as compared to a wild type plant grown under the same conditions. In an additional embodiment of this aspect, the conditions include non-steady light, optionally field conditions or fluctuating light. The growth conditions may be the fluctuating light of a crop canopy. In yet another embodiment of this aspect, which may be combined with any of the preceding embodiments, the genetically altered plant further includes increased yield as compared to the wild type plant grown under the same conditions.

### Molecular Biological Methods to Produce Genetically Altered Plants, Plant Parts, and Plant Cells

One aspect of the present disclosure provides genetically altered plants, plant parts, or plant cells with increased activity of one or more of a PPDK regulatory protein (PDRP), a Rubisco activase (Rca) protein, or a Rubisco protein that have increased photosynthetic efficiency under fluctuating light conditions. In addition, the present disclosure provides isolated DNA molecules of vectors and gene editing components used to produce genetically altered plants of the present disclosure.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); Wang, et al. Acta Hort. 461:401408 (1998), and Broothaerts, et al. Nature 433:629-633 (2005). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the compositions, methods, and processes disclosed herein. As an example, the CRISPR/Cas-9 system and related systems (e.g., TALEN, ZFN, ODN, etc.) may be used to insert a heterologous gene to a targeted site in the genomic DNA or substantially edit an endogenous gene to express the heterologous gene or to modify the promoter to increase or otherwise alter expression of an endogenous gene through, for example, removal of repressor binding sites or introduction of enhancer binding sites. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833-839); Gordon-Kamm et al., The Plant Cell, (1990) 2, 603-618), rice (Shimamoto et al., Nature, (1989) 338, 274-276; Datta et al., Bio/Technology, (1990) 8, 736-740), and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present disclosure can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal DNA or as modifications to an endogenous gene or promoter. Plants including the genetic alteration(s) in accordance with this disclosure include plants including, or derived from, root stocks of plants including the genetic alteration(s) of this disclosure, e.g., fruit trees or ornamental plants. Hence, any non-transgenic grafted plant parts inserted on a transformed plant or plant part are included in this disclosure.

Genetic alterations of the disclosure, including in an expression vector or expression cassette, which result in the expression of an introduced gene or altered expression of an endogenous gene will typically utilize a plant-expressible promoter. A ‘plant-expressible promoter’ as used herein refers to a promoter that ensures expression of the genetic alteration(s) of this disclosure in a plant cell. Examples of constitutive promoters that are often used in plant cells are the cauliflower mosaic (CaMV) 35S promoter (KAY et al. Science, 236, 4805, 1987), the minimal CaMV 35S promoter (Benfey & Chua, Science, (1990) 250, 959-966), various other derivatives of the CaMV 35S promoter, the figwort mosaic virus (FMV) promoter (Richins, et al., Nucleic Acids Res. (1987) 15:8451-8466)the maize ubiquitin promoter (CHRISTENSEN & QUAIL, Transgenic Res, 5, 213-8, 1996), the trefoil promoter (Ljubql, MAEKAWA et al. Mol Plant Microbe Interact. 21, 375-82, 2008), the vein mosaic cassava virus promoter (International Application WO 97/48819), and the Arabidopsis UBQ10 promoter, Norris et al. Plant Mol. Biol. 21, 895-906, 1993).

Additional examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871-2887), CabbB S(Franck et al., Cell (1980) 21, 285-294) and CabbB JI (Hull and Howell, Virology, (1987) 86, 482-493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the figwort mosaic virus (FMV) (Richins, et al., Nucleic Acids Res. (1987) 15:8451-8466), promoters of the Cassava vein mosaic virus (WO 97/48819; Verdaguer et al., Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S(GenBank accession numbers X04049, X00581), and the TR1′ promoter and the TR2′ promoter (the “TR1′ promoter” and “TR2′ promoter”, respectively) which drive the expression of the 1′ and 2′ genes, respectively, of the T DNA (Velten et al., EMBO J, (1984) 3, 2723-2730).

Alternatively, a plant-expressible promoter can be a tissue-specific promoter, i.e., a promoter directing a higher level of expression in some cells or tissues of the plant, e.g., in green tissues (such as the promoter of the chlorophyll a/b binding protein (Cab)). The plant Cab promoter (Mitra et al., Planta, (2009) 5: 1015-1022) has been described to be a strong bidirectional promoter for expression in green tissue (e.g., leaves and stems) and is useful in one embodiment of the current disclosure. These plant-expressible promoters can be combined with enhancer elements, they can be combined with minimal promoter elements, or can include repeated elements to ensure the expression profile desired.

Additional non-limiting examples of tissue-specific promoters include the maize allothioneine promoter (DE FRAMOND et al, FEBS 290, 103-106, 1991; Application EP 452269), the chitinase promoter (SAMAC et al. Plant Physiol 93, 907-914, 1990), the maize ZRP2 promoter (U.S. Pat. No. 5,633,363), the tomato LeExtl promoter (Bucher et al. Plant Physiol. 128, 911-923, 2002), the glutamine synthetase soybean root promoter (HIREL et al. Plant Mol. Biol. 20, 207-218, 1992), the RCC3 promoter (PCT Application WO 2009/016104), the rice antiquitine promoter (PCT Application WO 2007/076115), the LRR receptor kinase promoter (PCT application WO 02/46439), and the Arabidopsis pCO2 promoter (HEIDSTRA et al, Genes Dev. 18, 1964-1969, 2004). Further non-limiting examples of tissue-specific promoters include the RbcS2B promoter, RbcS1B promoter, RbcS3B promoter, LHB1B1 promoter, LHB1B2 promoter, cab1 promoter, and other promoters described in Engler et al., ACS Synthetic Biology, DOI: 10.1021/sb4001504, 2014. These plant promoters can be combined with enhancer elements, they can be combined with minimal promoter elements, or can include repeated elements to ensure the expression profile desired.

In some embodiments, further genetic alterations to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present disclosure can be inserted in host cell DNA so that the inserted gene part is upstream (i.e., 5′) of suitable 3′ end transcription regulation signals (i.e., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835-845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981-6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (i.e., detectable mRNA transcript or protein is produced) throughout subsequent plant generations. Stable integration into the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the term “overexpression” refers to increased expression (e.g., of mRNA, polypeptides, etc.) relative to expression in a wild type organism (e.g., plant) as a result of genetic modification and can refer to expression of heterologous genes at a sufficient level to achieve the desired result such as increased yield. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is upregulated. In some embodiments, an exogenous gene is upregulated by virtue of being expressed. Upregulation of a gene in plants can be achieved through any known method in the art, including but not limited to, the use of constitutive promoters with inducible response elements added, inducible promoters, high expression promoters (e.g., PsaD promoter) with inducible response elements added, enhancers, transcriptional and/or translational regulatory sequences, codon optimization, modified transcription factors, and/or mutant or modified genes that control expression of the gene to be upregulated in response to a stimulus such as cytokinin signaling.

Where a recombinant nucleic acid is intended for expression, cloning, or replication of a particular sequence, DNA constructs prepared for introduction into a host cell will typically include a replication system (e.g., vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present disclosure. One of skill in the art will recognize that any relevant markers available can be utilized in practicing the compositions, methods, and processes disclosed herein.

Screening and molecular analysis of recombinant strains of the present disclosure can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to this disclosure. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. See, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present disclosure can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the BLASTN, BLASTP, and BLASTX, programs of Altschul et al. (1990) J. Mol. Biol. 215:402-410. BLAST nucleotide searches are performed with the BLASTN program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (BLASTN and BLASTX) are used. See www.ncbi.nih.gov. One of skill in the art can readily determine in a sequence of interest where a position corresponding to amino acid or nucleic acid in a reference sequence occurs by aligning the sequence of interest with the reference sequence using the suitable BLAST program with the default settings (e.g., for BLASTP: Gap opening penalty: 11, Gap extension penalty: 1, Expectation value: 10, Word size: 3, Max scores; 25, Max alignments: 15, and Matrix: blosum62; and for BLASTN: Gap opening penalty: 5, Gap extension penalty:2, Nucleic match: 1, Nucleic mismatch-3, Expectation value: 10, Word size: 11, Max scores: 25, and Max alignments: 15).

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present disclosure can be introduced by any means known to the art which is appropriate for the particular type of cell, including without limitation, transformation, lipofection, electroporation or any other methodology known by those skilled in the art.

“Isolated”, “isolated DNA molecule” or an equivalent term or phrase is intended to mean that the DNA molecule or other moiety is one that is present alone or in combination with other compositions, but altered from or not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” from its natural setting within the scope of this disclosure so long as the element is not within the genome of the organism in which it is naturally found, the element is altered from its natural form, or the element is not at the location within the genome in which it is naturally found. Similarly, a nucleotide sequence encoding a protein or any naturally occurring variant of that protein would be an isolated nucleotide sequence so long as the nucleotide sequence was not within the DNA of the organism from which the sequence encoding the protein is naturally found in its natural location or if that nucleotide sequence was altered from its natural form. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant, alga, fungus, or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

Having generally described the compositions, methods, and processes of this disclosure, the same will be better understood by reference to certain specific examples, which are included herein to further illustrate the disclosure and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following example is offered to illustrate, but not to limit the claimed disclosure.

### Example 1: Development of a Dynamic Systems Model of C4 Photosynthesis

The following example describes the development of a dynamic systems model of C4 photosynthesis. This dynamic model was developed to capture the key factors affecting non-steady-state photosynthesis during transitions from low-light to high-light and vice-versa. Specifically, an existing C4 metabolic model for maize (for steady-state photosynthesis) was extended to include post-translational regulation of key photosynthetic enzymes, temperature responses of the enzyme activities, dynamic stomatal conductance, and leaf energy balance.

### Model Development

A generic dynamic systems model of C4 photosynthesis was developed from the previous NADP-ME metabolic model for maize (Wang, Y., et al. (2014) Elements Required for an Efficient NADP-Malic Enzyme Type C4 Photosynthesis. Plant Physiology, 164, 2231-2246; Wang, Y. et al. (2014) Three distinct biochemical subtypes of C4 photosynthesis? A modelling analysis. Journal of Experimental Botany, 65, 3567-3578). The NADP-ME metabolic model was an ordinary differential equation model including all individual steps in C4 photosynthetic carbon metabolism. This model was extended to include post-translational regulation and temperature response of enzyme activities, together with the dynamics of stomatal conductance and leaf energy balance. The model was implemented in MATLAB (The Mathworks, Inc*). Table 1, below, provides information regarding the parameters.

TABLE 1
Parameters

| Parameter | Full name | Unit |
|---|---|---|
| ϕ | CO₂ leakiness | Dimensionless |
| A | Net CO₂ uptake rate | μmol m⁻² s⁻¹ |
| Cᵢ | Intercellular CO₂ concentration | μbar |
| E | Transpiration rate | mol m⁻² s⁻¹ |
| f_{VmRubisco} | The ratio between measured V_{cmax} and the maximal Rubisco | Unitless |
| | activities in the model | |
| f_{VmPEPC} | The slope of the linear relationship between measured Vₚₘₐₓ and | Unitless |
| | the maximal PEPC activities in the model | |
| gₛ | Stomatal conductance | |
| gₛ_{_{—}}_{Kd} | Rate constant of Stomatal conductance decreasing | min⁻¹ |
| gₛ_{_{—}}_{Ki} | Rate constant of Stomatal conductance increasing | min⁻¹ |
| Jₘₐₓ | Maximum electron transport capacity | μmol m⁻² s⁻¹ |
| Kₒ | Michaelis-Menten constant of Rubisco for O₂ | mbar |
| K_{c} | Michaelis-Menten constant of Rubisco for CO₂ | μbar |
| PAR | Photosynthetically active photon flux | μmol m⁻² s⁻¹ |
| PEPC | PEP carboxylase | |
| PDRP | PPDK regulatory protein | |
| PPDK | Pyruvate, phosphate dikinase | |
| Rca | Rubisco activase | |
| R_{d} | Mitochondria respiration | μmol m⁻² s⁻¹ |
| τ_{Rubisco} | Time constant of Rubisco activation | min |
| V_{cmax} | Maximum rubisco activity estimated from measured A-CI curve | μmol m⁻² s⁻¹ |
| Vₚₘₐₓ | Maximum PEPC activity estimated from measured A-CI curve | μmol m⁻² s⁻¹ |
| Vₘₐₓ | Maximum activity of enzyme | μmol m⁻² s⁻¹ |
| WUE | water use efficiency (A/E) | mmol m⁻² |
| | | s⁻¹CO₂/mol m⁻² |
| | | s⁻¹ H₂O |

Post-translational regulation of enzyme activity; PPDK activation state

The pyruvate phosphate dikinase (PPDK) activity is regulated by the PPDK regulatory protein (PDRP) which is affected by the level of incident light via ADP concentration (Ashton, A. et al. (1984) Regulation of C4 photosynthesis: inactivation of pyruvate, Pi dikinase by ADP-dependent phosphorylation and activation by phosphorolysis. Archives of Biochemistry and Biophysics, 230, 492-503; Burnell, J. and Hatch, M. (1983) Dark-light regulation of pyruvate, Pi dikinase in C4 plants: evidence that the same protein catalyses activation and inactivation. Biochemical and biophysical research communications, 111, 288-293; Chastain, C. J. (2010) Structure, function, and post-translational regulation of C 4 pyruvate orthophosphate dikinase. In C4 Photosynthesis and Related CO2 Concentrating Mechanisms (Raghavendra, A. S. and Sage R. F., eds.) Dordrect: Springer, pp. 301-315). PDRP is a bifunctional protein kinase/protein phosphatase, catalyzing reversible phosphorylation of PPDK. The inactivation rate (V_{PDRP_I1}) and activation rate (V_{PDRP_A}) were calculated by the following equations:

$$\begin{matrix}{V}_{PDRP \_ I} = \frac{{[ PDRP ]}_{Mchl} \cdot {k}_{cat \_ PDRP \_ I} \cdot {[ E ]}_{Mcjl} \cdot {[ ADP ]}_{Mchl}}{( {[ E ]}_{Mchl} + {K}_{m \_ PPDK \_ PDRP \_ I} ) ( {[ ADP ]}_{Mchl} + {K}_{m \_ ADP \_ PDRP \_ I} ( 1 + \frac{{[ Pyr ]}_{Mchl}}{{K}_{i \_ Pyr \_ PDRP \_ I}} ) )} & ( 1 ) \\ {V}_{PDRP\_A} = \frac{{[ PDRP ]}_{Mchl} \cdot {k}_{cat \_ PDRP \_ A} \cdot {[ EP ]}_{Mcjl} \cdot {[ Pi ]}_{Mchl}}{( {[ EP ]}_{Mchl} + {K}_{m \_ PPDK \_ PDRP \_ A} ) \cdot ( ( 1 + \frac{{[ ADP ]}_{Mchl}}{{K}_{i \_ ADP \_ PDRP \_ A}} ) ) ( {[ Pi ]}_{Mchl} + {K}_{m \_ Pi \_ PDRP \_ A} )} & ( 2 )\end{matrix}$$

Where [PDRP]_{Mchl} is the PDRP concentration in the mesophyll cell chloroplasts, and k_{cat_PDRP_l} and k_{cat_PDRP_A} are the turnover number of PDRP for the inactivation and activation reaction respectively. [E]_{Mchl} is the concentration of active PPDK in the mesophyll chloroplasts, and [EP]_{Mchl} is the concentration of inactive PPDK in the mesophyll chloroplasts.

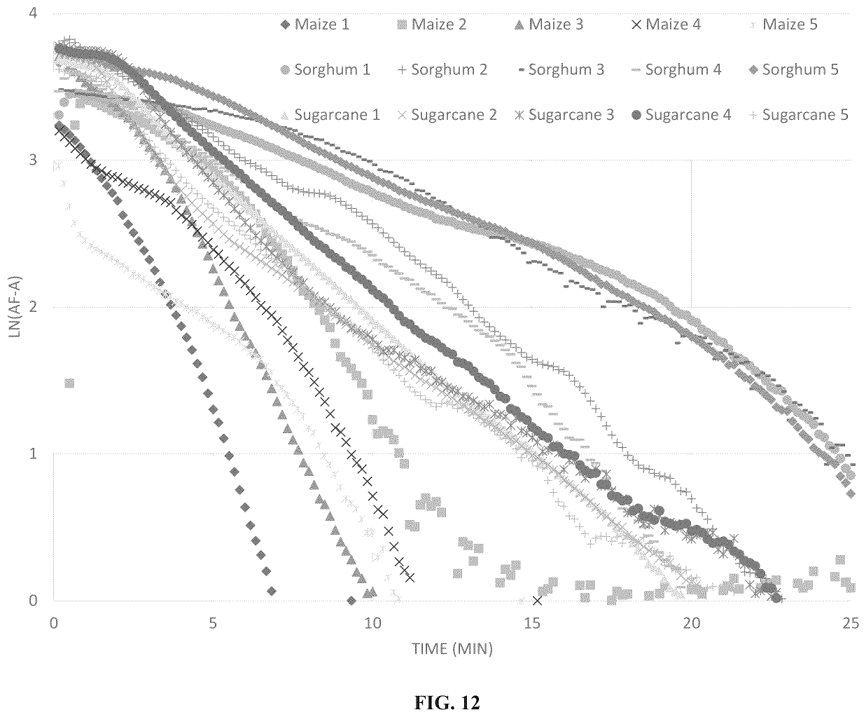

Post-translational regulation of enzyme activity; Rubisco activation state:
The time constant of Rubisco activation was determined from the measured kinetics of photosynthetic gas exchange (see Example 2) following transitions from dark to high light, using the method given by Woodrow and Mott (Woodrow, I. and Mott, K. (1989) Rate limitation of non-steady-state photosynthesis by ribulose-1, 5-bisphosphate carboxylase in spinach. Functional Plant Biology, 16, 487-500) (Eq. 27, FIG. 12). The differential equations of the transient maximal Rubisco activity is:

$$\frac{{dV}_{max \_ Rubisco \_ i}}{dt} = \frac{1}{{τ}_{Rubisco}} ( {V}_{max \_ Rubisco \_ s} - {V}_{max \_ Rubisco \_ i} ) \qquad ( 3 )$$

Where τ_{Rubisco} is the rate constant of Rubisco activation catalyzed by Rubisco activase. V_{max_Rubisco_i} is the transient maximal Rubisco activity; V_{max_Rubisco_s} is the steady-state maximal Rubisco activity which is related to the Rubisco activase concentration ([Rca]) (Mott, K. A. and Woodrow, I. E. (2000) Modelling the role of Rubisco activase in limiting non-steady-state photosynthesis. Journal of Experimental Botany, 51, 399-406). The total Rubisco activase concentration ([Rca]) is calculated using measured τ_{Rubisco} (Table 2, Eq. 25)

$$[ Rca ] = \frac{k}{{τ}_{Rubisco}} \qquad ( 4 )$$

Where k is a constant, which is 216.9 min mg m⁻²(Mott, K. A. and Woodrow, I. E. (2000) Modelling the role of Rubisco activase in limiting non-steady-state photosynthesis. Journal of Experimental Botany, 51, 399-406).

Steady-state maximal Rubisco activity is calculated with the following equations

$$\begin{matrix}{V}_{max \_ Rubisco \_ s} = \frac{{V}_{max \_ Rubisco} \cdot {[ Rca ]}_{A}}{{K}_{activase} + {[ Rca ]}_{A}} & ( 5 ) \\ {[ Rca ]}_{A} = [ Rca ] * {a}_{Rca \_ s} & ( 6 )\end{matrix}$$

where V_{max_Rubisco} is the theoretical maximum activity of Rubisco. [Rca]_{A} is the concentration of active Rubisco activase, which is regulated by light intensity (Section 1.3). K_{activase} is a constant, which equals 12.3 mg m⁻²(Mott, K. A. and Woodrow, I. E. (2000) Modelling the role of Rubisco activase in limiting non-steady-state photosynthesis. Journal of Experimental Botany, 51, 399-406).
Post-translational regulation of enzyme activity; Activation of enzymes regulated via light intensity

The model used a simplified equation for light regulation of ATP synthase (ATPase), sedoheptulose-1:7-bisphosphatase (SBPase), fructose-1:6-bisphosphatase (FBPase), phosphoribulose kinase (PRK), glyceraldehyde-3-phosphate dehydrogenase (GAPDH), and rubisco activase (Rca)

$$\begin{matrix}\frac{{dV}_{max \_ E \_ i}}{dt} = \frac{1}{{τ}_{E}} ( {V}_{max \_ E \_ s} - {V}_{max \_ E \_ i} ) & ( 7 ) \\ {a}_{E \_ s} = min ( ( {k}_{E \_ A} \cdot I + {c}_{E \_ A} ) , 1 ) & ( 8 ) \\ {V}_{max \_ E \_ s} = {V}_{max \_ E} \cdot {a}_{E \_ s} & ( 9 )\end{matrix}$$

Where V_{max_E_i} is the transient maximal enzyme activity, τ_{E} is the rate constant of the activation of each enzyme, and V_{max_E_s} is the steady-state maximal enzyme activity, as affected by light intensity (I). k_{E_A} and C_{E_A} are two constants, i.e. the slope and intercept of the linear relationship of the proportion of activated enzyme (a_{E_s}) as a function of I. V_{max_E} is the activity of the enzyme when fully activated.

Although activation of PEPC is regulated by light via phosphorylation, the whole pathway and parameters of this regulation have not been quantitatively measured. Thus, the dynamics of PEPC activity were as described by Eq. 7-9.

### Temperature Response of Enzymes

In order to simulate the effects of fluctuating leaf temperature with fluctuations in light, the Arrhenius equation (Johnson, F. H. et al. (1942) The nature of enzyme inhibitions in bacterial luminescence: sulfanilamide, urethane, temperature and pressure. Journal of Cellular and Comparative Physiology, 20, 247-268) and Q₁₀, function were used to adjust the enzymatic parameters to the actual leaf temperature (T_{leaf}). The formula used for each parameter was determined based on the availability of experimental data.

Temperature response of the maximum activity of carbonic anhydrase (CA) and PEP carboxylase (PEPC) (V_{max_CA} and V_{max_PEPC}) were incorporated into the model using a peaked Arrhenius Function (Johnson, F. H. et al., 1942).

$${V}_{max \_ Enz 1} = {V}_{max \_ Enz 1 \_ 25} \cdot {e}^{\frac{{E}_{a} \cdot ( {T}_{leaf} - 25 )}{298.15 \cdot R \cdot ( {T}_{leaf} + 273.15 )}} \cdot \frac{1 + {e}^{\frac{( 298.15 \cdot Δ S - {H}_{d} )}{298.15 \cdot R}}}{q + {e}^{\frac{( ( {T}_{leaf} + 273.15 ) \cdot Δ S - {H}_{d} )}{( {T}_{leaf} + 273.15 ) \cdot R}}} \qquad ( 10 )$$

Where Eₐ is the exponential rate of rise, H_{d} describes the rate of decrease at supra-optimal temperatures, and ΔS is the entropy factor.

Temperature response of enzymatic parameters of pyruvate phosphate dikinase (V_{max_PPDK}), electron transport capacity (Jₘₐₓ) and Rubisco (V_{max_Rubisco_Co2}, V_{max_Rabisco_O2}/V_{max_Rubisco_CO2}, Kₒ and K_{c}) were incorporated into the model using an Arrhenius Function.

$${V}_{max \_ Enz 2} = {V}_{max \_ Enz 2 \_ 25} \cdot {e}^{\frac{{E}_{a} \cdot ( {T}_{leaf} - 25 )}{298.15 \cdot R \cdot ( {T}_{leaf} + 273.15 )}} \qquad ( 11 )$$

For other enzymes, a Q₁₀ function was used to estimate the temperature response of the maximum activity, as described previously (Woodrow, I. E. and Berry, J. (1988) Enzymatic regulation of photosynthetic CO₂, fixation in C3 plants. Annual Review of Plant Physiology and Plant Molecular Biology, 39, 533-594). Q₁₀ was set as 2.

$${V}_{max \_ Enz 3} = {V}_{max \_ Enz 3 \_ 25} \cdot {{Q}_{10 \_ Enz}}^{\frac{( {T}_{leaf} - 25 )}{10}} \qquad ( 12 )$$

### Dynamic Stomatal Response

Ball-Berry model parameters for predicting steady-state stomatal conductance (Ball, J. et al. (1987) A model predicting stomatal conductance and its contribution to the control of photosynthesis under different environmental conditions. In Progress in photosynthesis research (Biggins, J. ed: Springer, Dordrech, pp. 221-224) were obtained from light response curves measured for each C4 crop evaluated in this disclosure. In the Ball-Berry model, stomatal conductance was with a function of A, relative humidity (RH) and CO₂ concentration at the leaf surface (C):

$${g}_{s \_ steady} = {Slope}_{BB} \frac{A \cdot RH}{{C}_{a}} + {Intercept}_{BB} \qquad ( 13 )$$

Where Slope_{BB} is the slope of the relationship between g_{s_steady} and A*RHs/Ca. Intercept_{BB} is the residual stomatal conductance. Slope_{BB} and Intercept_{BB} were estimated by linear regression of

$$\frac{A \cdot RH}{{C}_{a}}$$

and g_{s_steady} from light response curve (A-Q curve) measurement. Dynamic stomatal conductance (gₛ) was estimated by the following equation:

$$\frac{{dg}_{s}}{dt} = k ( {g}_{s \_ steady} - {g}_{s} ) \qquad ( 14 )$$

where g_{s_steady} is the steady-state stomatal conductance calculated by the Ball-Berry model (Eq.13) (Ball J. et al., 1987),k(kᵢ or k_{d}) is the rate constant of stomata conductance response calculated from measured stomata dynamics of the three C4 crops, kᵢ and k_{d} represent the rate constant of stomata conductance increasing and decreasing, respectively (Eq. 26). Table 2, below, provides the input parameters for the model. The values used were either collected from the literature or calculated from gas exchange measurements (see Example 2).

TABLE 2
Input parameters for the dynamic C4 photosynthesis model.

| | Original | | | | | Method of |
|---|---|---|---|---|---|---|
| Parameters | value | Reference | Maize | Sorghum | Sugarcane | measurement |
| Slope_{BB} | 4.53 | Miner et al., 2017 | 5.183 ± 0.419 | 4.843 ± 0.211 | 4.941 ± 0.177 | A-Q curve |
| Intercept_{BB} | 0.020 | Miner et al., 2017 | 0.036 ± 0.020 | 0.019 ± 0.013 | 0.027 ± 0.007 | A-Q curve |
| gₛ_{_{—}}ₖᵢ | 0.227 | McAusland et al., | 0.127 ± 0.016 | 0.257 ± 0.063 | 0.204 ± 0.031 | Shade-light |
| (min⁻¹) | | 2016 | | | | dynamics |
| gₛ_{_{—}}_{kd} | 0.071 | McAusland et al., | 0.123 ± 0.026 | 0.377 ± 0.055 | 0.221 ± 0.023 | Shade-light |
| (min⁻¹) | | 2016 | | | | dynamics |
| Vₚₘₐₓ (μmol | 120 | Von Caemmerer | 124.128 ± 9.253 | 133.626 ± 5.678 | 83.840 ± 4.140 | A-Ci curve |
| m⁻² s⁻¹) | | 2000 | | | | |
| V_{cmax} (μmol | 60 | Von Caemmerer | 49.919 ± 1.847 | 51.082 ± 2.001 | 52.783 ± 1.975 | A-Ci curve |
| m⁻² s⁻¹) | | 2000 | | | | |
| τ_{Rubisco} | 5 | Woodrow and | 3.881 ± 1.117 | 9.714 ± 2.338 | 4.776 ± 0.316 | Dark-light |
| (min) | | Mott 1989 | | | | dynamics |
| f_{VmPEPC} | 1 | assumed | 0.72 | 0.68 | 0.92 | A-Ci curve |
| f_{VmRubisco} | 0.85 | assumed | 0.67 | 0.56 | 0.67 | A-Ci curve |
| [PDRP] | 0.04 | assumed | 0.058 | 0.038 | 0.037 | Dark-light |
| (μmol) | | | | | | dynamics |
| Rd(μmol | 1 | Von Caemmerer | 2.282 | 0.979 | 1.446 | Dark-light |
| m⁻² s⁻¹) | | 2000 | | | | dynamics |

### Dynamic Leaf Energy Balance

For leaf energy balance, the equations used in the model were based on the method of Nikolov et al. (Nikolov, N. T. et al. (1995) Coupling biochemical and biophysical processes at the leaf level: an equilibrium photosynthesis model for leaves of C3 plants. Ecological Modelling, 80, 205-235). According to this model, leaf energy balance takes account of intercepted short- and long-wave radiation, radiative energy loss from the leaf, convection, and latent heat loss in transpiration. The net photosynthesis rate (A), stomatal conductance and leaf temperature are interdependent. For example, A affects stomatal conductance, stomatal conductance affects leaf temperature and leaf temperature affects A. Instead of solving these steady-state circular connections iteratively (Nikolov, N. T. et al., 1995), differential equation describes leaf temperature (T_{leaf}) change (Eq. 15)

$$\frac{{dT}_{leaf}}{dt} = \frac{{PAR}_{abs} + NIR + LR - ( H + LE + E + Me )}{{C}_{p} \cdot {m}_{leaf}} \qquad ( 15 )$$

$$H = 2 {C}_{{p}_{- air}} {g}_{bh} ( {T}_{leaf} - {T}_{air} ) \qquad ( 16 )$$

$$LE = \frac{{C}_{lv} {g}_{l}}{{P}_{a}} ( {E}_{sat} - {E}_{air} ) \qquad ( 17 )$$

$$E = 2 ϵσ {T}_{leafK}^{4} \qquad ( 18 )$$

$${M}_{e} = 0.506 A \qquad ( 19 )$$

Where PAR_{abs} is absorbed photosynthetic active radiation, assuming 85% of PAR absorbed by leaf, NIR is absorbed near-infrared radiation, and LR is absorbed long wave radiation. Both NIR and LR were set to zero. C, is specific heat capacity of leaf, here it was assumed that it is the same as the specific heat capacity of water (4.184 J g⁻¹° C.⁻¹). m_{leaf} is the specific leaf fresh weight (g m⁻²), here it was set as 198 g m⁻² for all species based on measured value of maize leaves (197.9±4.5 g m²). Humidity in the leaf internal air space was assumed to be saturated at the temperature of the leaf. H and LE are the sensible and latent heat fluxes, respectively. E is the emitted long wave radiation, and Me is the energy consumed in photosynthesis (Nikolov, N. T. et al., 1995). The boundary layer conductance to heat is calculated as g_{bh}=0.924_{gb} (Nikolov, N. T. et al., 1995). C_{p_air} is the specific heat capacity of air (29.3 J mol⁻¹° C.⁻¹), Cₗᵥ is the latent heat of vaporization of water (44000 J mol-¹), gᵢ is the total conductance of the stomata and the boundary layer, ϵ is the leaf emissivity of long wave radiation, and σ is the Boltzman constant.

### Boundary Layer Conductance

Boundary layer conductance was calculated following Nikolov et al. (1995), both free and forced convection was considered in determining the boundary layer conductance of leaf. The leaf boundary layer conductance to vapor transport is the maximum of g_{bf} and g_{br}

$${g}_{b} = max ( {g}_{bf} , {g}_{br} ) \qquad ( 20 )$$

Forced-convective and free-convective leaf boundary layer conductance is computed as

$${g}_{bf} = {c}_{f} {{{T}_{airk}}^{0.56} [ ( {T}_{airk} + 120 ) \frac{u}{{d}_{o} {P}_{a}} ]}^{0.5} \qquad ( 21 )$$

$${g}_{br} = {c}_{e} {{T}_{leafk}^{0.56} ( \frac{{T}_{leafK} + 120}{{P}_{a}} )}^{0.5} {( Δ T )}^{0.25} \qquad ( 22 )$$

where dₒ is the characteristic dimension of a leaf (leaf width), ΔT is the temperature difference between leaf and the local air (Monteith and Unsworth, 1990), u is the wind velocity, and c_{ƒ} and cₑ are two constants.

### Model Prediction

### CO₂ Uptake Rate (A) and Leakiness (φ) Calculation

During the simulation, metabolite concentrations and reaction rates were extracted from the model. The velocity of CO₂ flowing into the leaf via stomata was used to represent A. Leakiness (ϕ) describes the proportion of carbon fixed by PEP carboxylase (PEPC) that subsequently leaks out of the bundle sheath cells. Thus, ϕ was calculated as:

$$ϕ = \frac{{v}_{CO 2 \_ leak}}{{v}_{PEPC}} = \frac{{P}_{CO 2\_ pd} ( {[ CO 2 ]}_{BSC} - {[ CO 2 ]}_{MC} )}{{v}_{PEPC}} \qquad ( 30 )$$

Where the CO₂ leak rate (ν_{CO2_leak}) is determined by the permeability of CO₂ through plasmodesmata (P_{CO2_pd}) and the concentration gradient of CO₂ between bundle sheath cytosol and mesophyll cytosol ([CO2]BSC−[CO2],_{MC}), and ν_{PEPC} is the velocity of carbon fixation by PEPC.

### Sensitivity Analysis

Sensitivity coefficient (SCₚ) gives the relative fractional change in simulated result with fractional change in input variable (p), SCₚ is the partial derivative used to describe how the output estimate varies with changes in the values of the input parameter (p), the output in this disclosure is estimated leaf CO2 uptake rate (A):

$${SC}_{p} = \frac{\partial A}{\partial p} \frac{p}{A} \approx \frac{{A}^{+} - {A}^{-}}{0.02 \cdot A} \qquad ( 31 )$$

where the variable (p) was both increased and decreased by 1% individually in the model to calculate the new A (A⁺ and A⁻) to identify the parameters influencing A.

Flux control coefficient of each enzyme (FCC) was also estimated by Eq. 31, using the maximal activity (V_{max_E}) of the enzyme as the variable (p).

### Example 2: Gas Exchange Measurement and Parameter Estimation

The following example describes gas exchange measurements of the three C4 crops maize, sugarcane, and sorghum. The gas exchange data obtained was used to parameterize the model of Example 1.

### Materials and Methods

Gas exchange measurements of Zea mays (maize) B73, Saccharum oficinarum (sugarcane) CP88-1762, and Sorghum bicolor (sorghum) Tx430 were used to calculate the values of following photosynthetic parameters: maximum Rubisco activity, maximum PEP carboxylase activity, the rate constants for stomatal conductance during opening and closing, time constants for Rubisco activation, mitochondrial respiration, concentration of PPDK regulatory protein, and the Ball-Berry slope and intercept (Table 2).

### Plant Material and Growth Conditions

Maize B73, sugarcane CP88-1762, and sorghum Tx430 were grown in a controlled environment greenhouse at 28° C. (day)/24° C. (night). Maize and sorghum were grown from seed, and sugarcane CP88-176 was grown from stem cuttings. Plant positions in the greenhouse were re-randomized every week to avoid the influence of environmental variations within the greenhouse. From July 25 to Aug. 8, 2019, six biological replicates were measured in a randomized experimental design for each species in each measurement.

### Steady-State Gas Exchange Measurements and Parameter Estimation

Leaf gas exchange of the youngest fully expanded leaf was measured on 30 to 35 day-old plants with a gas exchange system (LI-6800; LI-COR, Lincoln, NE, USA). The leaf chamber temperature was set a 28° C., a water vapor pressure deficit of 1.32 KPa and the flow rate at 500 μmol s⁻¹ for all the gas exchange measurements.

For the response of A to intracellular CO₂ concentration curves (A-C, curves), the leaf was acclimated to a light intensity of 1800 μmol m²s¹ and a CO₂ concentration of 400 μmol mol⁻¹. After both A and gₛ reached steady-state, the CO₂ concentration of the influent gas was varied in the following sequence: 400, 300, 200, 120, 70, 40, 20, 10, 400, 400, 400, 600, 800, 1200 and 1500 μmol mol⁻¹.

The maximum Rubisco activity (V_{cmax}) and maximum PEP carboxylase activity (Vₚₘₐₓ) were estimated from the A-Cᵢ curves using the equations from Von Caemmerer (Von Caemmerer, S. (2000) Biochemical models of leaf photosynthesis: Csiro publishing.). In order to obtain the relationship between estimated Vₚₘₐₓ and the theoretical maximal PEPC activity (V_{max_PEPC}) in the model, similarly, the relationship between V_{cmax} and the theoretical maximal Rubisco activity (V_{max_Rubisco}), two variables (ƒᵥₚₘₐₓ and ƒ_{vcmax}) were introduced into the simulation:

$${V}_{max \_ PEPC} = \frac{1}{{f}_{vpmax}} {V}_{pmax} \qquad ( 23 )$$

$${V}_{max \_ Rubisco} = \frac{1}{{f}_{vcmax}} {V}_{cmax} \qquad ( 24 )$$

ƒᵥₚₘₐₓ and ƒ_{vcmax} were estimated by minimizing the sum (S_{fvPEPC} and S_{fvcmax}) of squared difference between the dynamic model estimated A (A_{e_ci}) and measured A (A_{m_ci}) response to intercellular CO₂ (A-Cᵢ curve) using least squares method for each species.

$${S}_{fvPEPC} = {( {s}_{Ae \_ Ci} ( {f}_{vPEPC} ) - {s}_{Am \_ Ci} )}^{2} \qquad ( 25 )$$

$${S}_{fvcmax} = \sum {( {A}_{e \_ Ci} ( {f}_{vcmax} ) - {A}_{m \_ Ci} )}^{2} \qquad ( 26 )$$

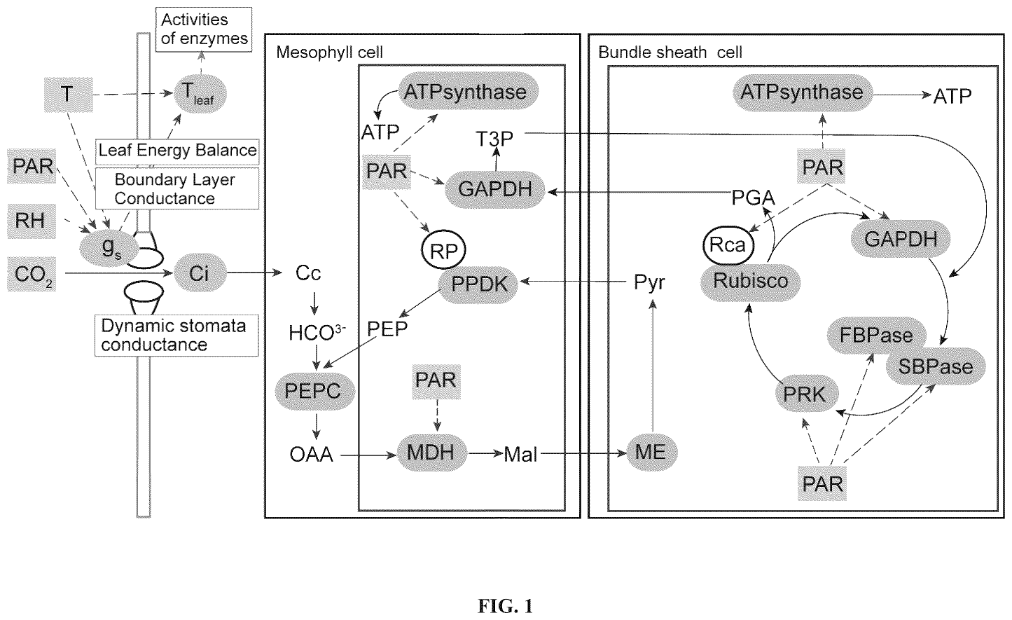

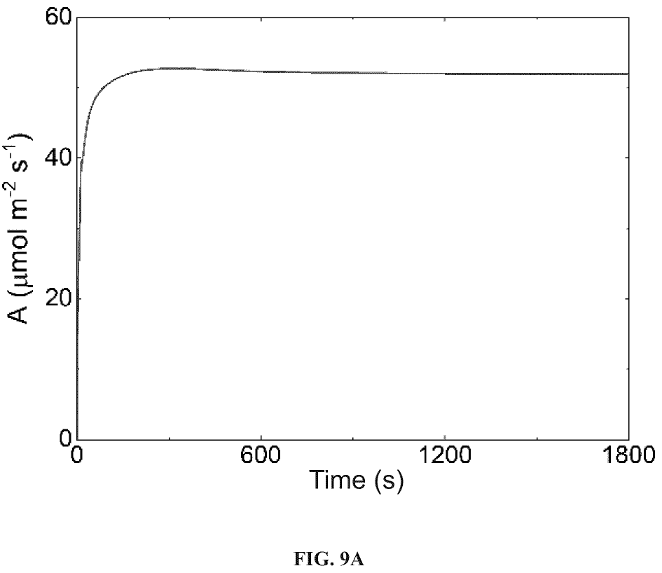

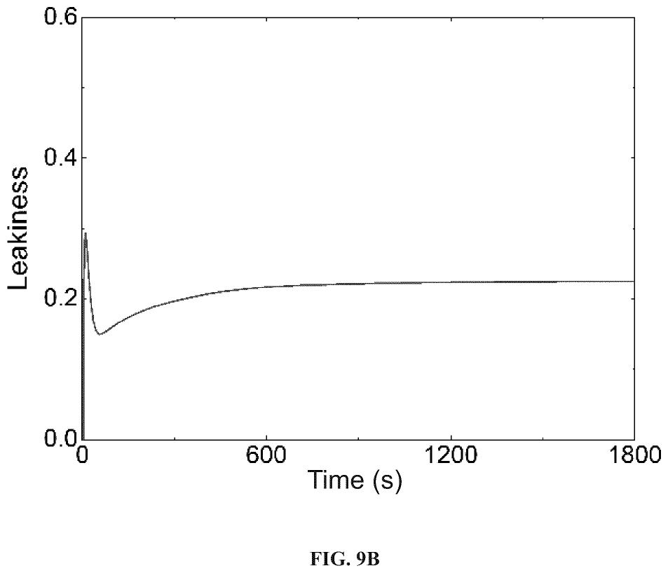

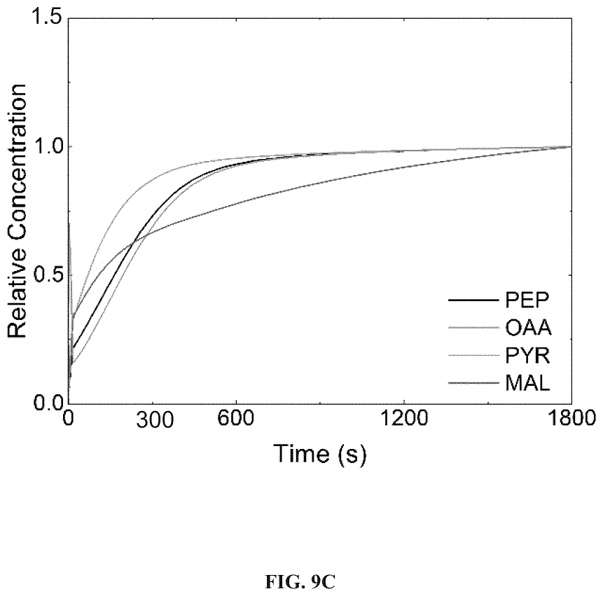

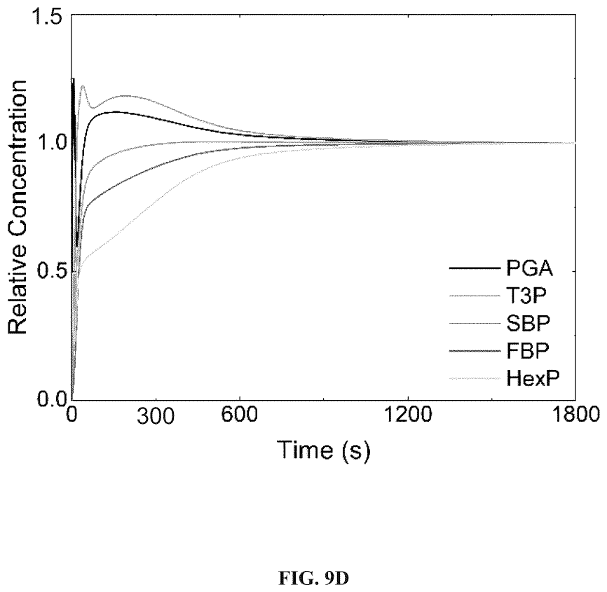

ƒ_{vPEPC} was estimated using the initial slope (S_{Am_Ci}) of measured A-Cᵢ curve (CO₂ air=120, 70, 40, 20, 10 μmol mol⁻¹); the ƒ_{vcmax} was estimated using CO₂ saturated A_{m_ci} (CO₂ air=800, 1200 and 1500 μmol mol⁻¹) (FIG. 9). The steady-state Vₘₐₓ of the other enzymes of C4 and C3 metabolism of FIG. 1 were scaled for each species with ƒᵥₚₘₐₓ and ƒ_{vcmax}, respectively.

To define the response of A to light intensity (A-Q curves), the leaf was acclimated to a light intensity of 1800 μmol m⁻²s⁻¹ and a CO₂ concentration of 400 μmol mol⁻¹. After leaf gas exchange reached steady-state, the light intensity in the chamber was changed in the following sequence: 2000, 1500, 1000, 500, 300, 200, 100 and 50 μmol m² s- The gas exchange data were logged after 5 min to ensure there was enough time for the transpiration, and therefore stomatal conductance, to reach steady state at each light level. Ball-Berry model parameters (Ball, J., Woodrow, I. and Berry, J. (1987) A model predicting stomatal conductance and its contribution to the control of photosynthesis under different environmental conditions. In Progress in photosynthesis research Biggins, J. ed: Springer, Dordrech, pp. 221-224) were estimated by linear regression of

$$\frac{A \cdot RH}{{C}_{a}}$$

and g_{s_steady} from data of A-Q curves, including to predict steady state stomatal conductance (g_{s_steady}) for each species (Eq.13).

### Dynamic Gas Exchange Measurements and Parameter Estimation

Gas exchange during photosynthetic induction was measured in the transition from darkness to high light (1800 μmol m⁻²s⁻¹) to determine the kinetics of Rubisco activation in these C4 crops (τ_{Rubisco}). The leaf was first acclimated to darkness for 30 min, with CO₂ concentration of 400 μmol mol⁻¹, the light intensity was then changed to 1800 smol m⁻² s⁻¹ for 30 min, which was more than sufficient time for leaf CO₂ uptake and stomatal conductance to reach steady-state. Leaf gas exchange was logged before the light was turned on, and then logged every 10 s for the following 30 min. The time constant of Rubisco activation (τ_{Rubisco}) was estimated from the linear portion of the semi-logarithmic plot of photosynthesis with time (Woodrow, I. and Mott, K. (1989) Rate limitation of non-steady-state photosynthesis by ribulose-1, 5-bisphosphate carboxylase in spinach. Functional Plant Biology, 16, 487-500; Woodrow, I. E. and Mott, K. A. (1993) Modelling C3 photosynthesis: A sensitivity analysis of the photosynthetic carbon-reduction cycle. Planta, 191, 421-432), FIG. 12). The slope of this portion was determined by linear regression of the data between 3 and 7 min. The value of τ_{Rubisco} was calculated as:

$${τ}_{Rubisco} = - \frac{1}{slope} \qquad ( 27 )$$

Calculated values of the three C4 species are listed in Table 2.

To further evaluate the response of gas exchange in C4 plants under fluctuating light, following this 30 min of induction, responses to the transition from high to low and back to high light (i.e. relaxation curves followed by induction curves) were measured. This involved decreasing light to 200 μmol m-²s−1 PPFD for 30 minutes then returning to 1800 μmol m⁻²s⁻¹ PPFD for an additional 30 minutes. Gas exchange was recorded every 10 s.

Rate constants were calculated for gₛ increase on transfer from low light (200 μmol m⁻²s⁻¹ PPFD) to high light (kᵢ), and again for the decrease in gₛ on return to low light (k_{d}). Measured time series for stomatal conductance changes were fit to the following equation (Vialet-Chabrand, S. R. et al. (2017) Temporal dynamics of stomatal behavior: modeling and implications for photosynthesis and water use. Plant physiology, 174, 603-613):

$${g}_{s} = ( {g}_{max} - {g}_{0} ) {e}^{- kt} + {g}_{0} \qquad ( 28 )$$

where gₘₐₓ is the maximum stomata conductance, g₀ is the minimum stomata conductance, t is time, and k(kᵢ or k_{d}) is the rate constant of gₛ, gₘₐₓ, g₀ and k were estimated using Eq. 28 by the curve fitting function (fit) in MATLAB™ (The Mathworks, Inc).

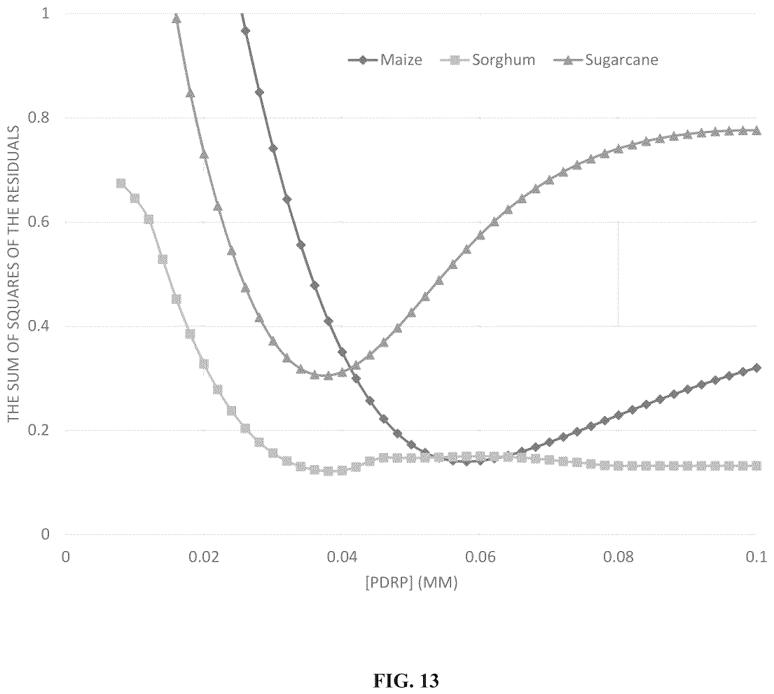

Mitochondrial respiration (R_{d}) was estimated from the measured CO₂ efflux after 30-minute dark adaptation. The PPDK regulatory protein (PDRP) concentration was estimated by minimizing difference between dynamic model estimated A (A_{e_t}) and measured A(A_{m_t}) in the beginning of the induction using least squares method, which minimizes the sum (S_{PDRP}) of squared the difference between estimated and measured A in the beginning of the photosynthetic induction (FIG. 13), data points between 1-3 minute of the induction was used

$${S}_{PDRP} = \sum {( {A}_{e \_ t} ( [ PDRP ] ) - {A}_{e \_ t} )}^{2} \qquad ( 29 )$$

### Model Parameterization

The model took the following 11 photosynthetic parameters estimated from measured gas exchange data as input variables: maximum Rubisco activity (V_{cmax} and ƒ_{vcmax}), maximum PEP carboxylase activity (Vₚₘₐₓ and ƒᵥₚₘₐₓ), the rate constant of stomata conductance increase and decrease (kᵢ, k_{d}), time constant of rubisco activation (τ_{Rubisco}), mitochondrial respiration (R_{d}), concentration of PPDK regulatory protein ([PDRP]), the Ball-Berry slope (Slope_{BB}) and intercept (Intercept_{BB}) (Table 2). The estimation methods of the input variables were described in Example 2 (Gas exchange measurement and parameter estimation).

### Results

### Measured Photosynthetic Induction of Maize, Sorghum, and Sugarcane

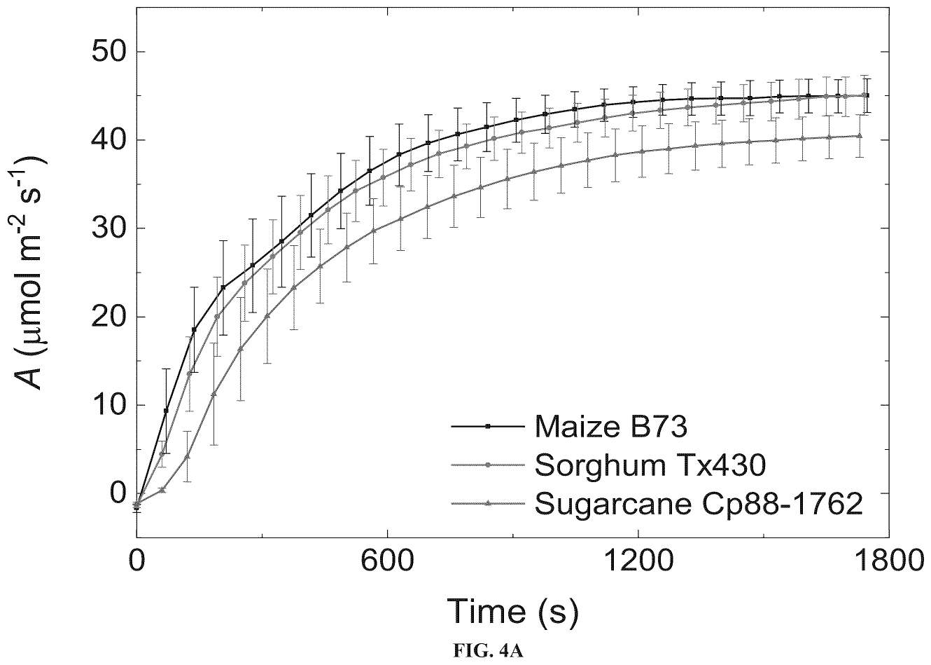

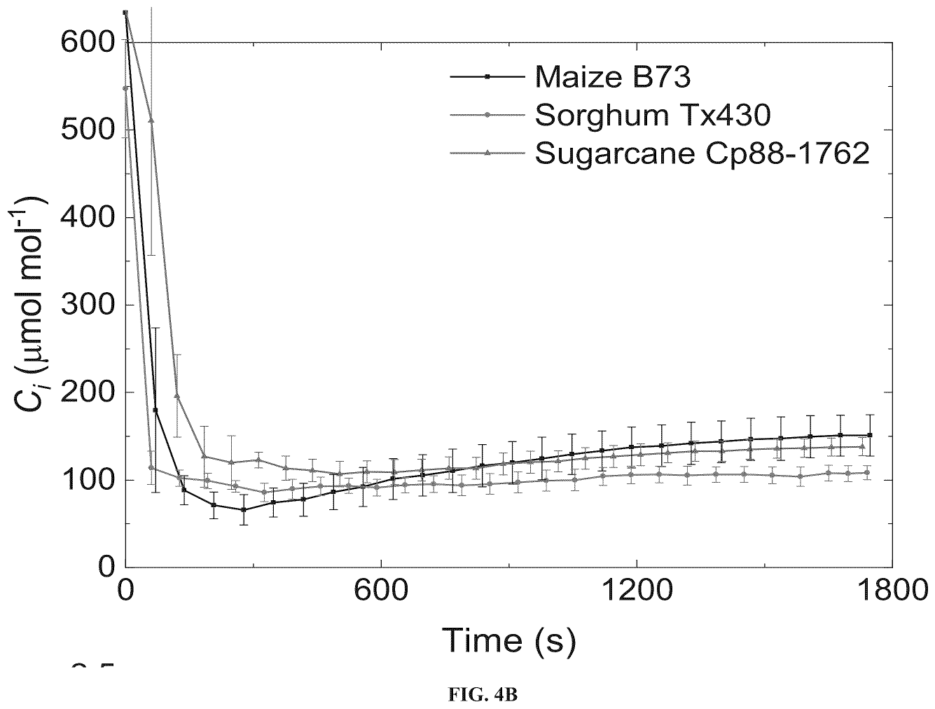

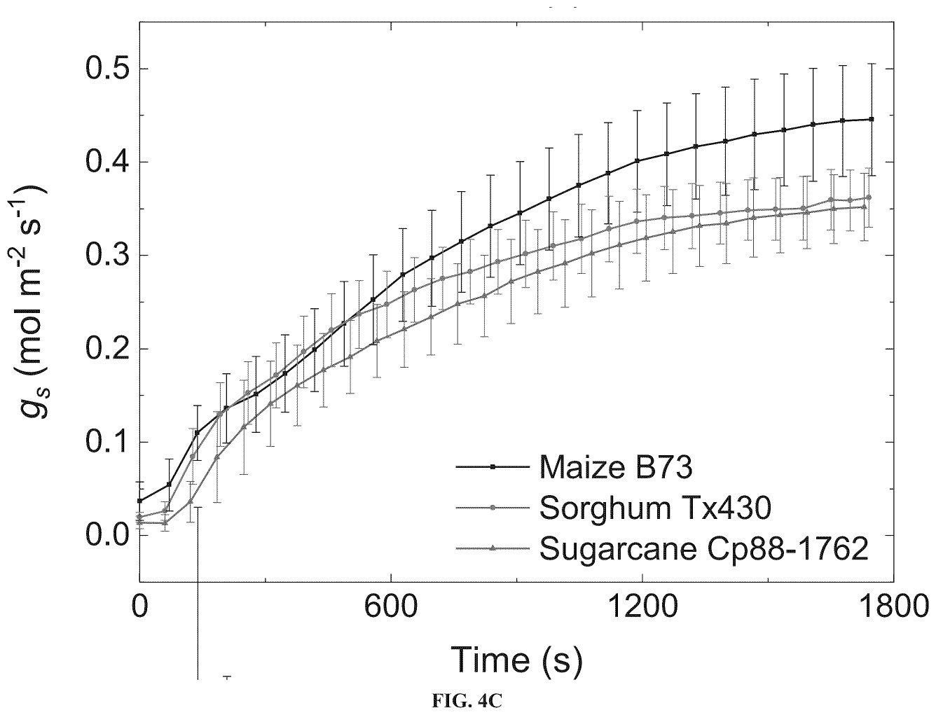

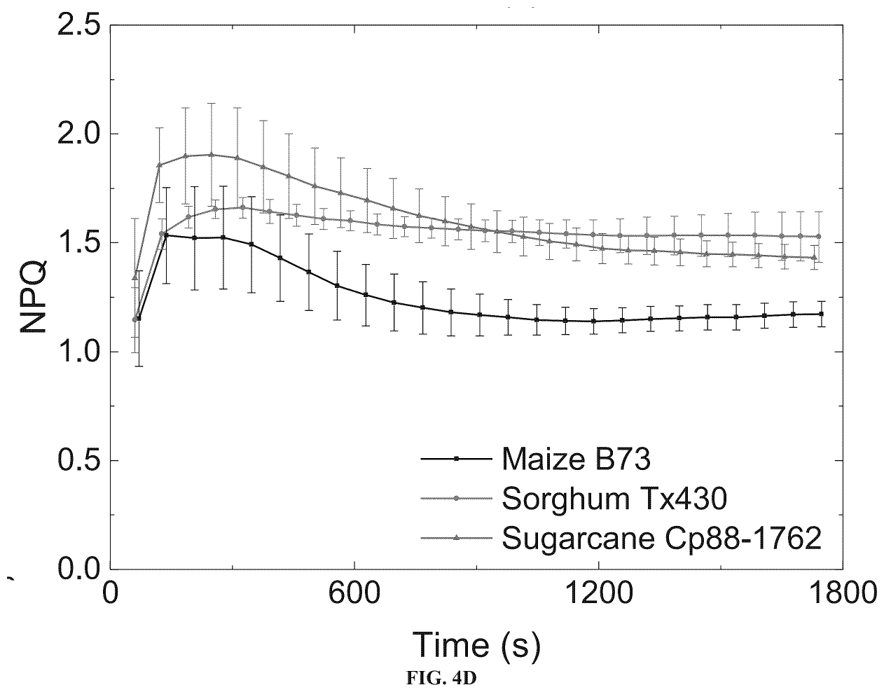

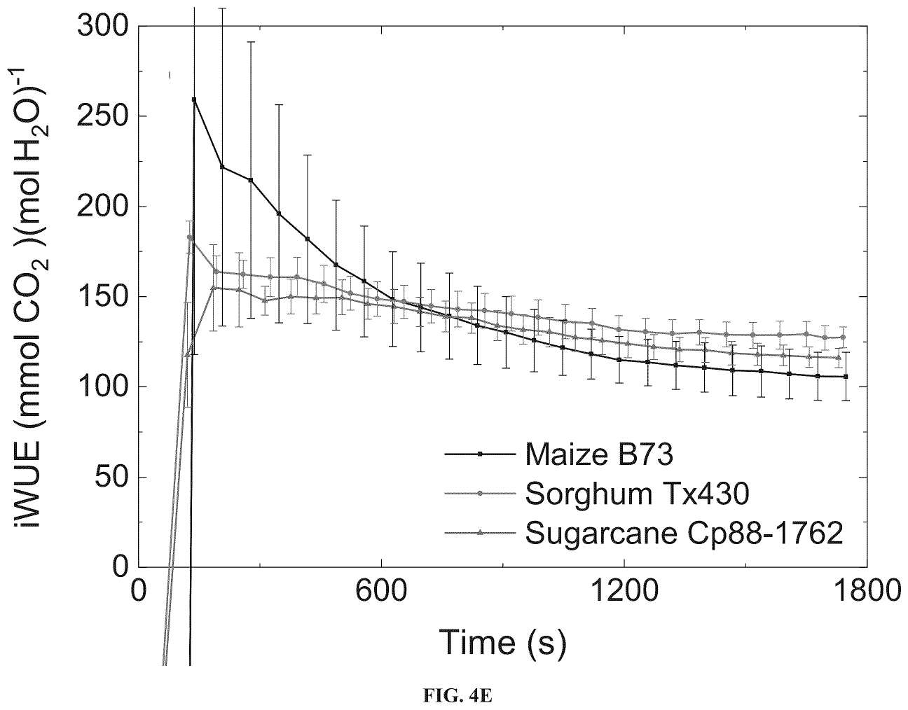

To further analyze the limitations for different C4 crop species, steady-state and dynamic gas exchange of three major C4 crops were measured, using one widely grown or well-studied genotype for each species, i.e., maize B73, sugarcane CP88-1762, and sorghum Tx430. During the dark to high light transition, the CO₂ assimilation rate of maize rose the fastest, followed by sorghum, and then sugarcane (FIG. 4A). The time required to reach 50% of the steady-state rate (IT50) for the three crops was 196 s, 237 s and 316 s respectively. The average CO₂ assimilation rate in the 30-minutes induction reduced by 17.7%, 20.6% and 24.2% in maize, sorghum and sugarcane, respectively, compared to the steady-state CO₂ assimilation rate. However, maize had a slower rate of increase in gₛ compared to sorghum and sugarcane (FIG. 4C and Table 2). Intercellular CO₂ concentration (C) dropped rapidly in the first ca. 100 s, then slowly increased to the steady-state level. The lowest C, was 66 μmol mol⁻¹, 86 μmol mol⁻¹ and 107 μmol mol⁻¹ in maize, sorghum and sugarcane respectively, which are 53%, 21% and 22% lower compared to their steady-state CG values (FIG. 4B). The low Cᵢ values would appear insufficient to fully saturate photosynthesis from about 180 s to 600 s after illumination began. Maize showed the highest intrinsic water use efficiency (iWUE) in the first 600 s, whereas, sorghum had highest iWUE after 600 s (FIG. 4E). iWUE is the ratio of the rate of CO2 assimilation (A) to the stomatal conductance (gₛ). Non-photochemical quenching (NPQ) of the three species rose to a peak at ca. 60s and then declined to a steady-state at ca. 600s largely in concert with assimilation (FIG. 4D).

### Model Parameterization and Validation

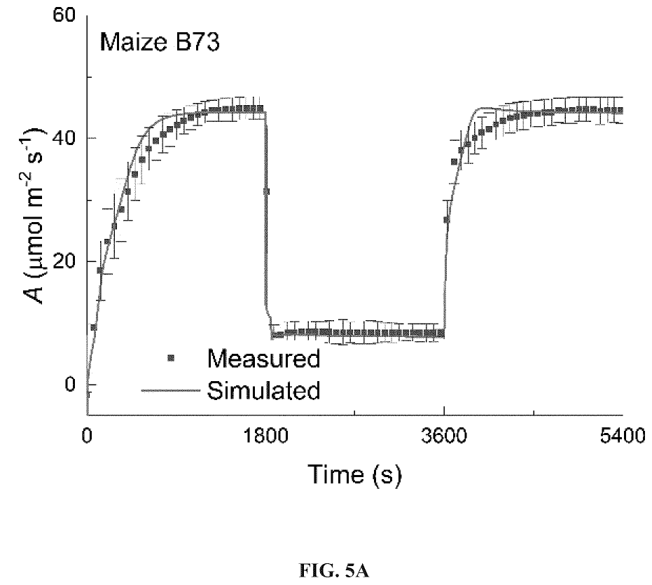

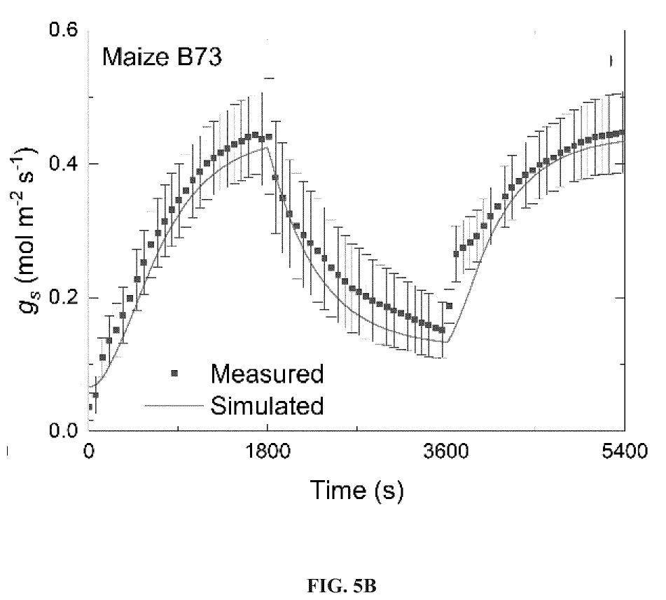

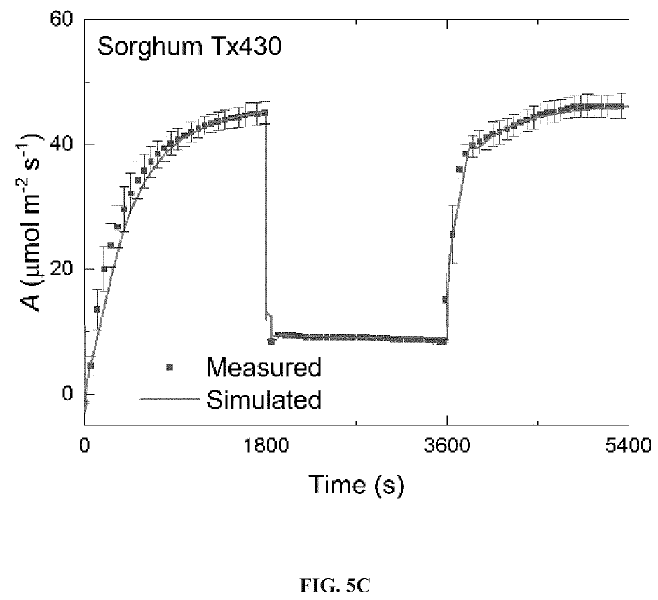

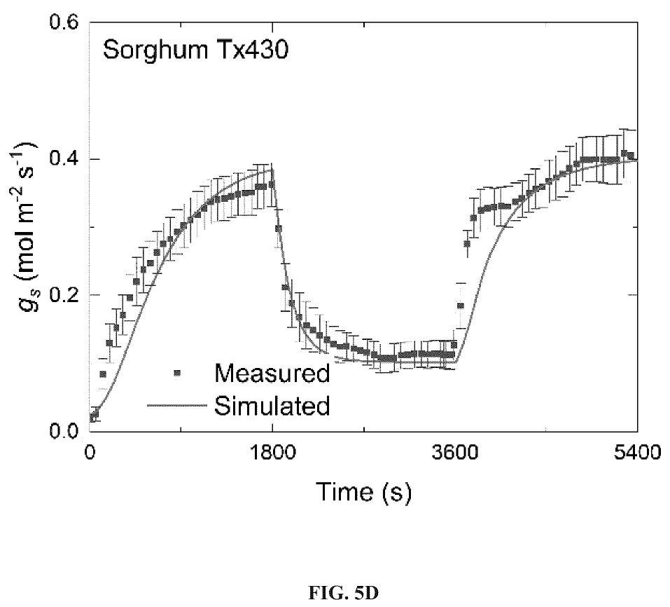

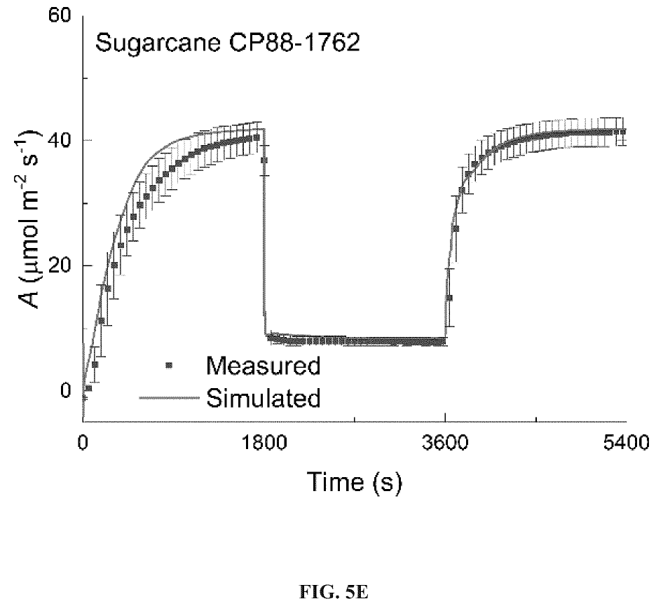

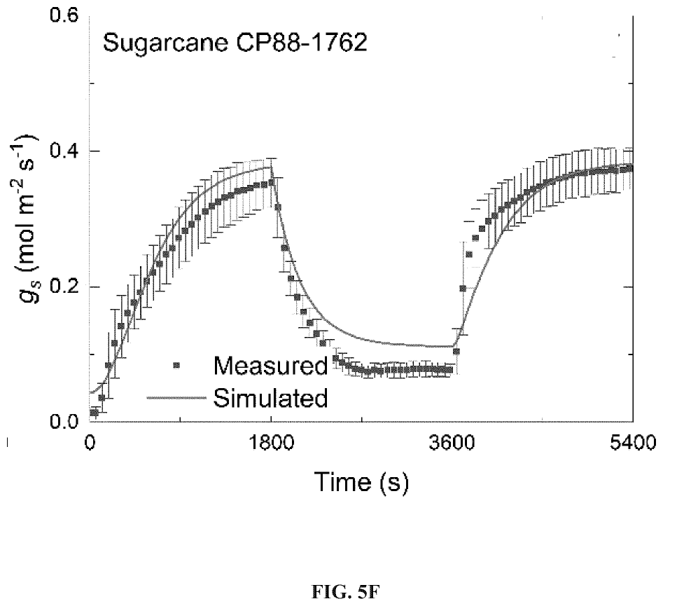

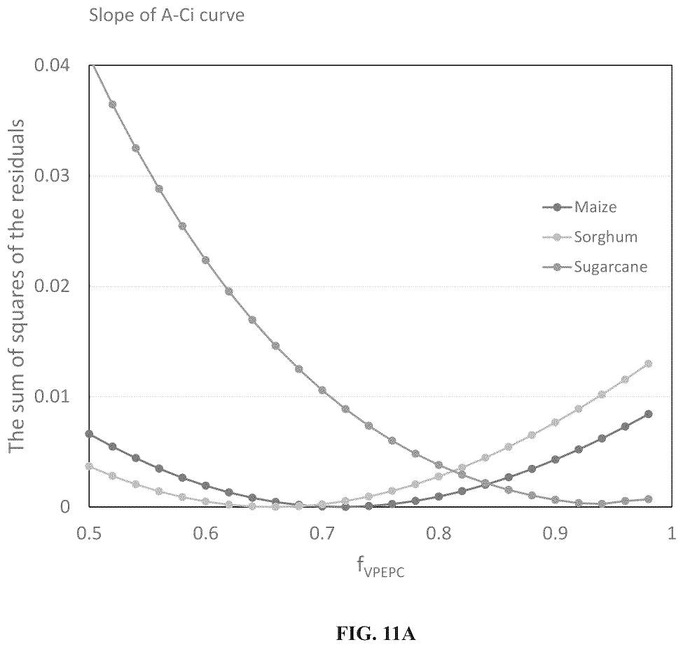

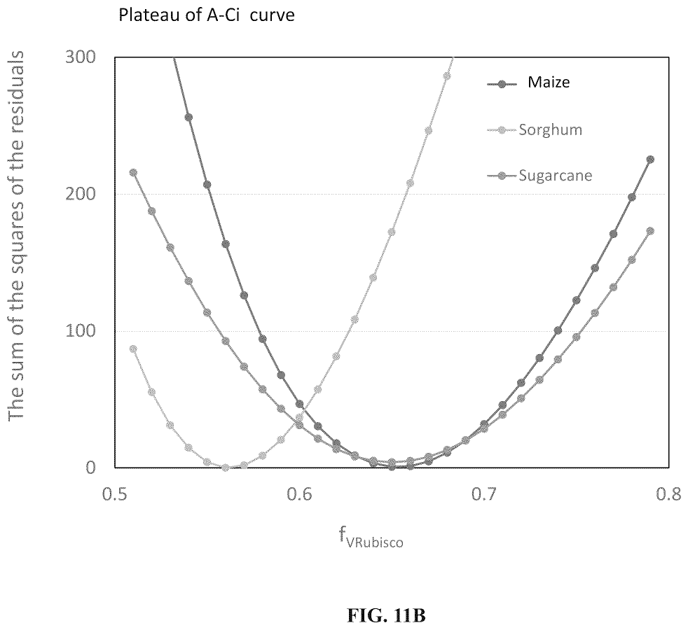

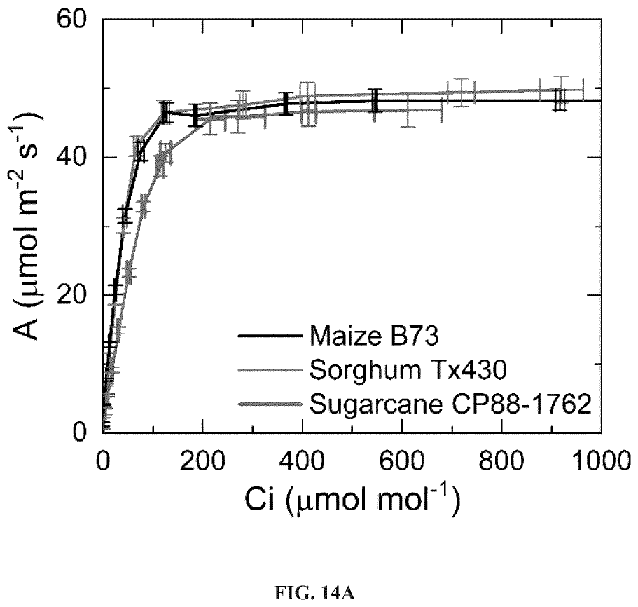

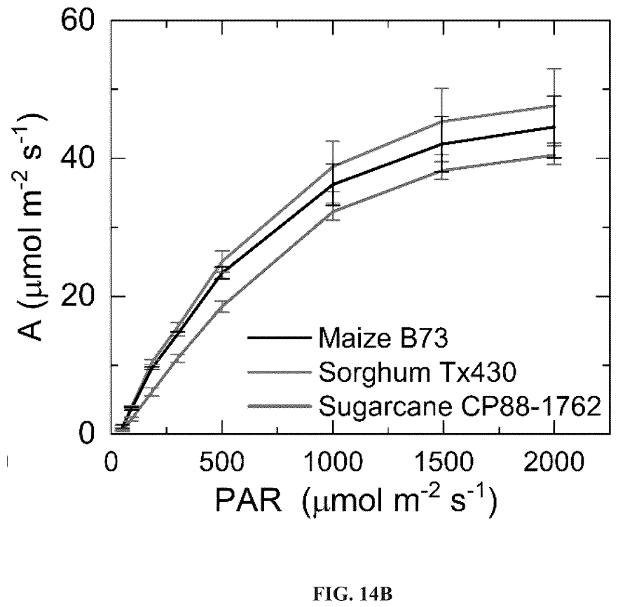

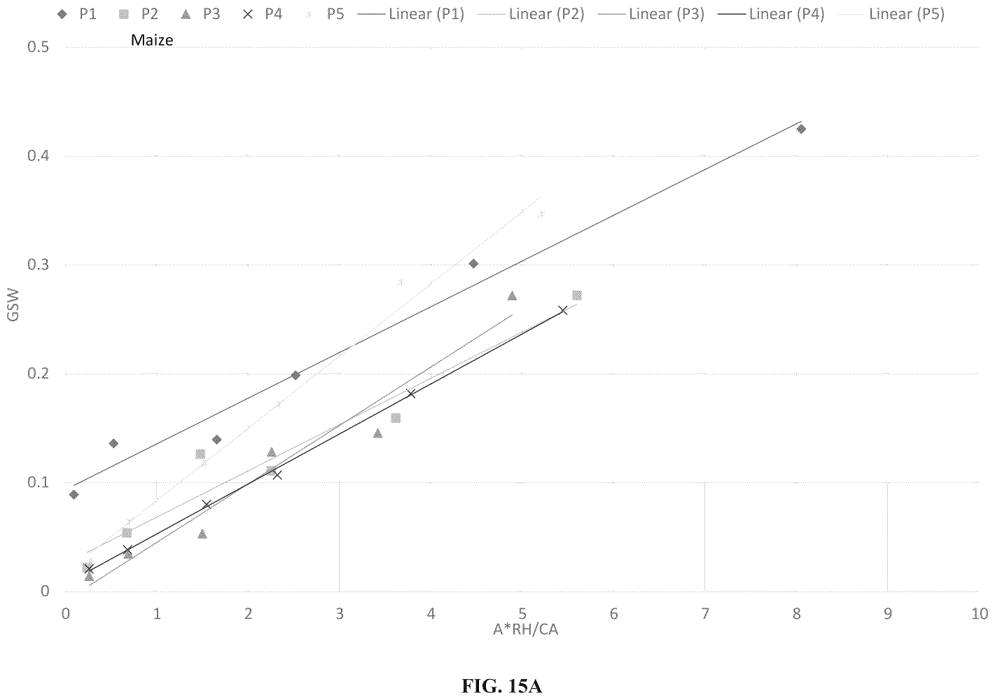

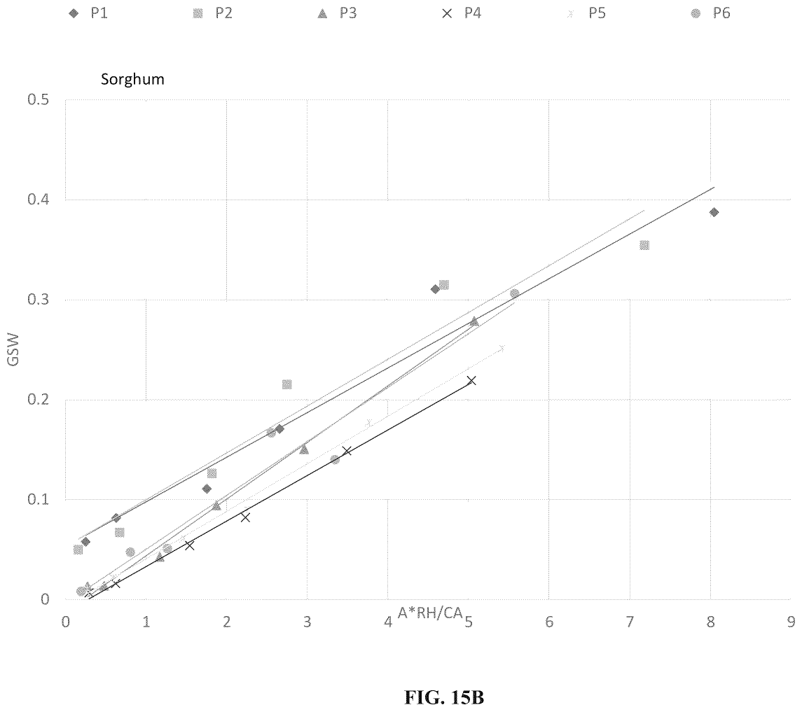

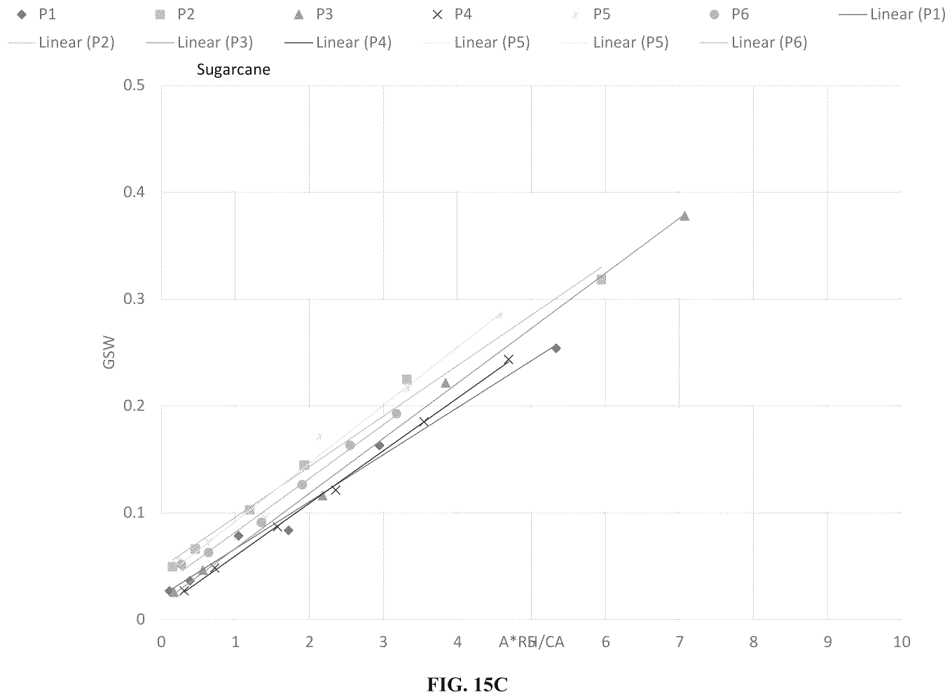

Using the measured steady-state and dynamic gas exchange data (FIGS. 14A-14B, 4A-4E, and 5A-5F), the following photosynthetic parameters were estimated: maximum Rubisco activity (V_{cmax}), maximum PEP carboxylase activity (Vₚₘₐₓ), the rate constants for stomatal conductance on dark-light and light-dark transitions, respectively (kᵢ, k_{d}), time constant of Rubisco activation (τ_{Rubisco}), mitochondrial respiration (R_{d}), concentration of PPDK regulatory protein ([PDRP]), and the Ball et al. (1987) model slope and intercept (Table 2; FIGS. 11A-11B, 12, 13, 14A-14B, and 15A-15C). With these species-specific parameters alone, the model was able to closely replicate the measured dynamics of A and gₛ in all three C4 crops under fluctuating light (FIGS. 5A-5F). This consisted of 30-min dark adaptation, followed by 30-minute intervals of high light, low light and high light again (FIGS. 5A-5F).

### Example 3: Use of the Dynamic Systems Model of C4 Photosynthesis to Identify Limitations to C4 Photosynthesis in Fluctuating Light

The following example describes the results obtained from using the model developed in Example 1 for simulations. Initially, values from the literature were used for simulations, and subsequently, the model was parameterized with experimental values for simulations (see Example 2). Further, this example provides discussion of these results.

### Results

### Factors Influencing Induction of C4 Photosynthesis on Dark-High Light Transitions

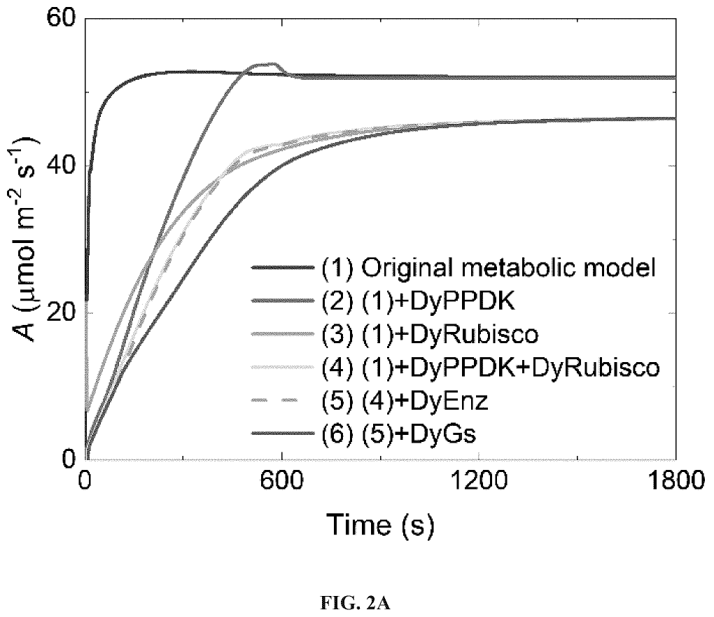

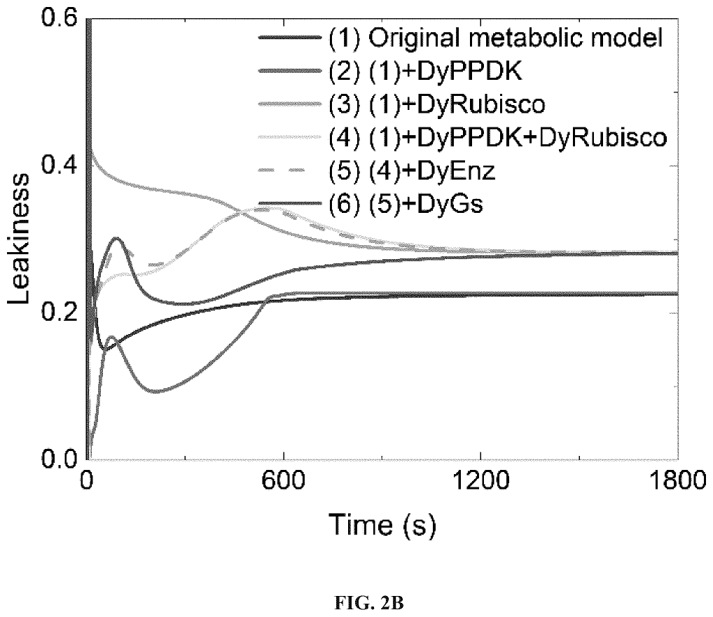

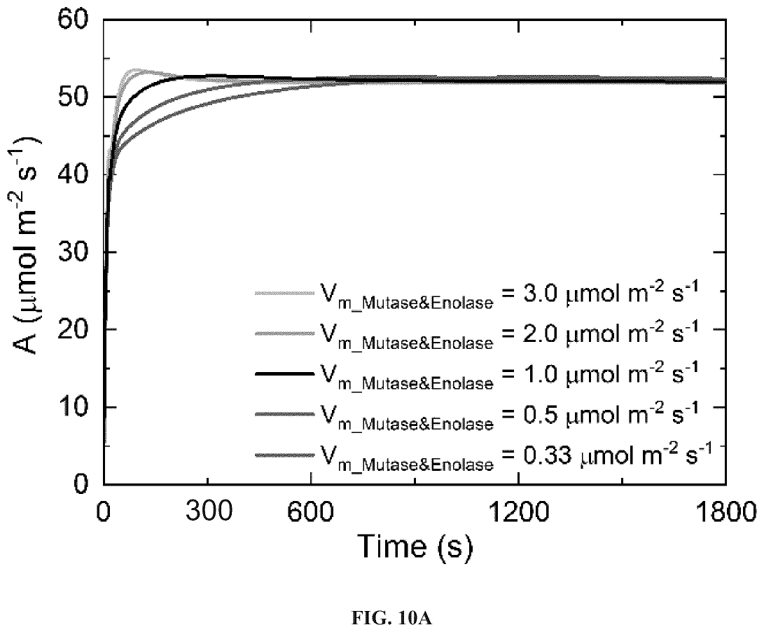

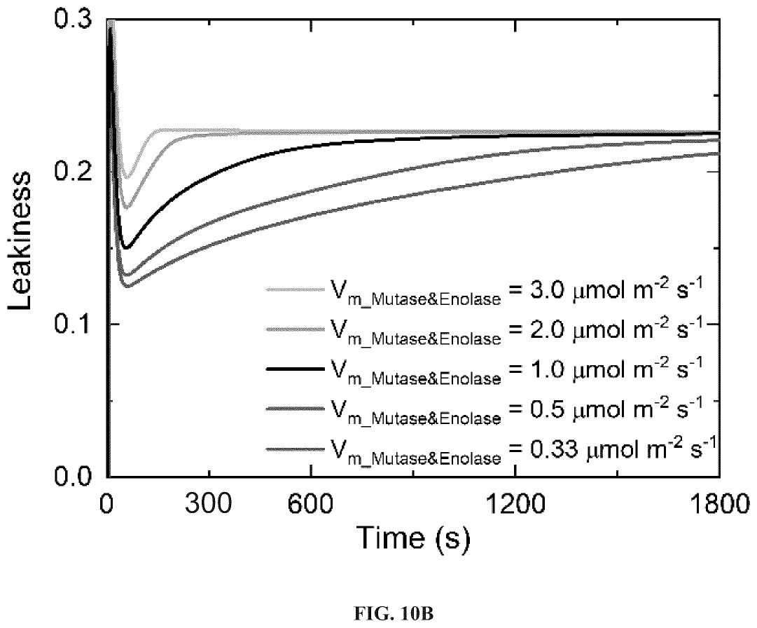

The new dynamic model of Example 1 extended a C4 metabolic model (Wang, Y. et al. (2014) Elements Required for an Efficient NADP-Malic Enzyme Type C4 Photosynthesis. Plant Physiology, 164, 2231-2246) to include post-translational regulation and temperature responses of enzymes, dynamic stomatal conductance and leaf energy balance (FIG. 1). The model was built by superimposing dynamic regulation of enzyme activation and stomatal conductance on the metabolic NADP-ME C4 leaf photosynthesis model of Wang et al. (2014). This was initially parameterized from the literature (Table 2). During induction some C4 metabolic pools, in particular malate in the bundle sheath cytoplasm rise to very high concentrations (Leegood, R. C. (1997) The regulation of C-4 photosynthesis. In Advances in Botanical Research 26, 251-316). To assess the role of this accumulation of photosynthetic metabolites during induction, the model was first run assuming that all enzymes were fully activated and the stomata open. Termed Scenario 1, this resulted in a rapid induction to near-steady-state within 120s (FIG. 2A). The major limitation over this period was the time taken for C4 metabolites to accumulate and approach steady-state, lagging C3 metabolites (FIGS. 9A-9D). Leakiness (ϕ), i.e. the proportion of CO₂ released by decarboxylation in the bundle sheath that diffused back to the mesophyll, reached a minimum at 30 s, gradually climbing to steady-state value of ca. 0.22 at ca. 600 s, indicating that the flux through the C4 cycle continued to limit photosynthesis (FIG. 2B). This limitation was affected by the activity of mutase and enolase, the enzymes that convert PGA to PEP. Increasing the maximum activity of mutase and enolase accelerated induction in Scenario 1 (FIGS. 10A-10B).

In Scenario 2, regulation of PPDK by its regulatory protein (PDRP) substantially slowed the rate of induction (dA/dt) of A (FIG. 2A) by limiting PEP synthesis, thus lowering the predicted ϕ(FIG. 2B). In Scenario 3, Rubisco regulation alone is added and resulted in a similar decrease in the rate of induction of (dA/dt), to that of Scenario 2. It reduced the final steady-state A, since a greater proportion of Rubisco now remained inactive (FIG. 2A). As would be expected in contrast to Scenario 2, leakiness is high in Scenario 3 throughout induction, since the C4 cycle is delivering CO₂ to the bundle sheath, but Rubisco is not fully activated and so less able to utilize the CO₂ being released by malate decarboxylation (FIG. 2B). Combining both activation of PPDK and Rubisco to give Scenario 4 results in a yet slower rate of induction (FIG. 2A), but the closer co-ordination of activation of the two enzymes results in less bundle-sheath leakiness during induction. The simulated leakiness increases for the first 600 s and then declines to a steady-state value of ca. 0.28 by 1200 s, reflecting a predicted faster activation of PPDK than Rubisco (FIG. 2B). The addition of the dynamic control of the other light activated enzymes of photosynthetic carbon metabolism (FIG. 1) in Scenario 5 produces dynamic responses of A and ϕ almost identical to that of Scenario 4 (FIGS. 2A-2B). Finally, superimposing the response of stomatal conductance in Scenario 6 on the dynamics of A and Cᵢ further slows the speed of induction, but dampens the bundle sheath leakage that would otherwise occur (FIGS. 2A-2B).

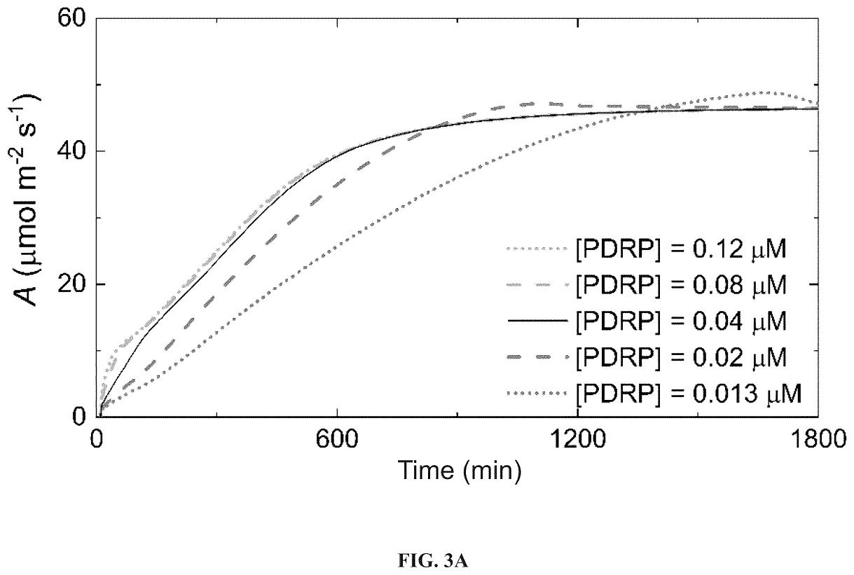

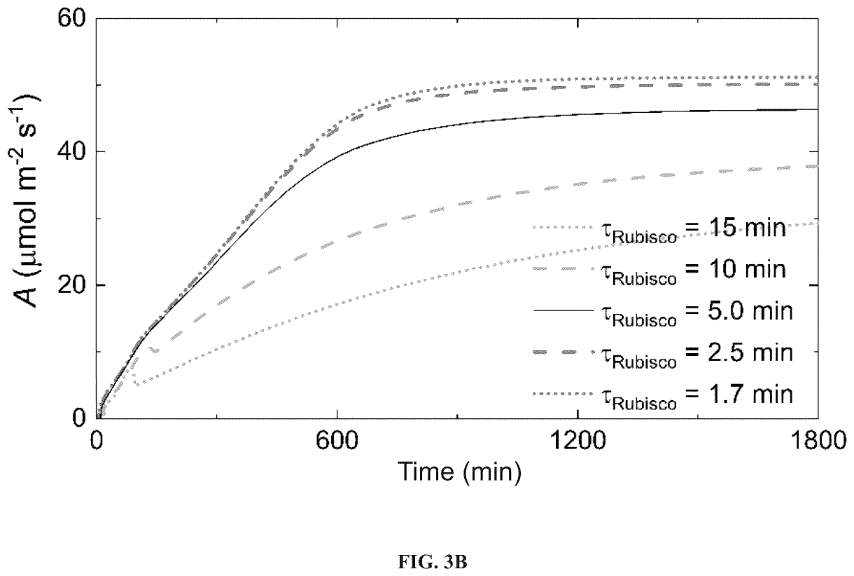

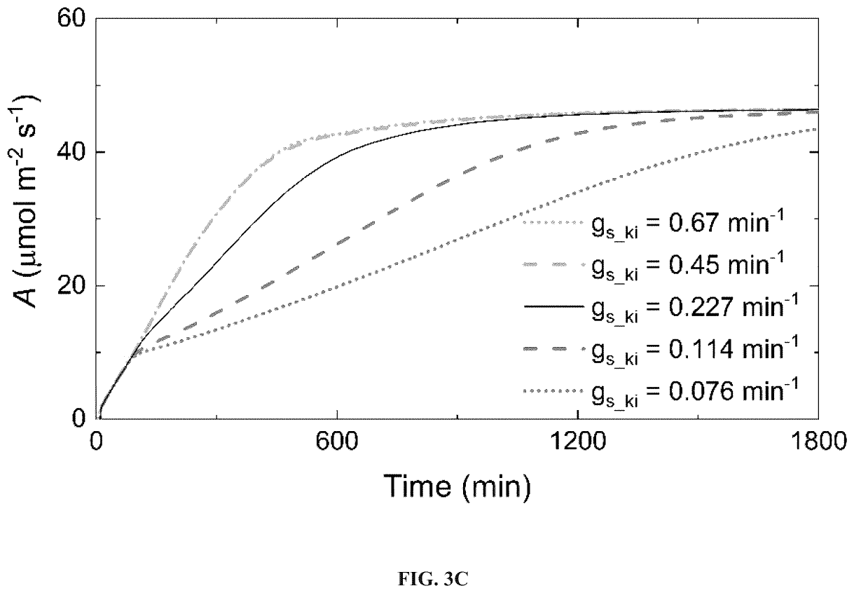

The model is shown to predict typical dynamic responses of A and gₛ, both with respect to pattern and magnitude during induction. The simulation predicts PPDK activation, Rubisco activation, and stomatal dynamics as the major limitations, while activation of other enzyme of carbon metabolism and metabolic pool size adjustment had little effect (FIGS. 3A-3C). The concentration of PDRP regulates the initial phase of the photosynthetic induction curve (FIG. 3A); while the speed of Rubisco activation affects the later phase of the induction (FIG. 3B). During the mid-phase of induction, gₛ is shown to limit A (FIG. 3C).

### Model Parameterization and Validation Using Experimental Values

Model parameterization and validation using experimental values was done as described in Example 2.

### Factors Limiting the Speed of Photosynthetic Induction

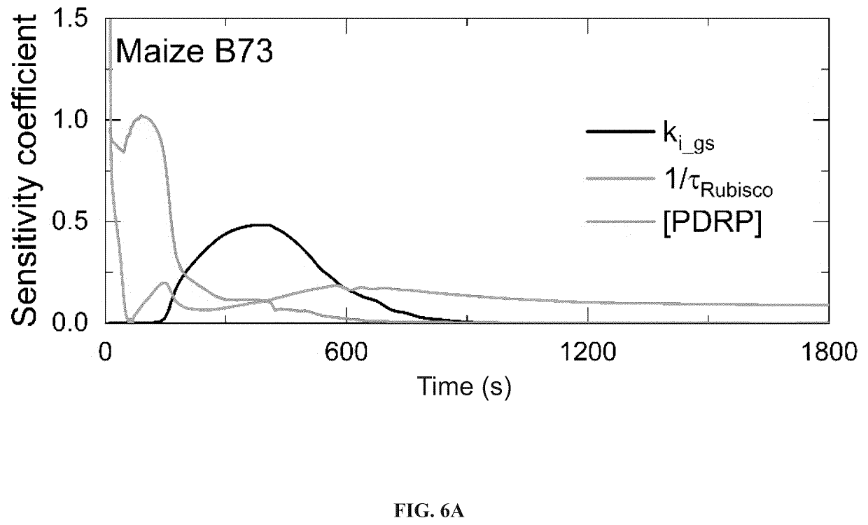

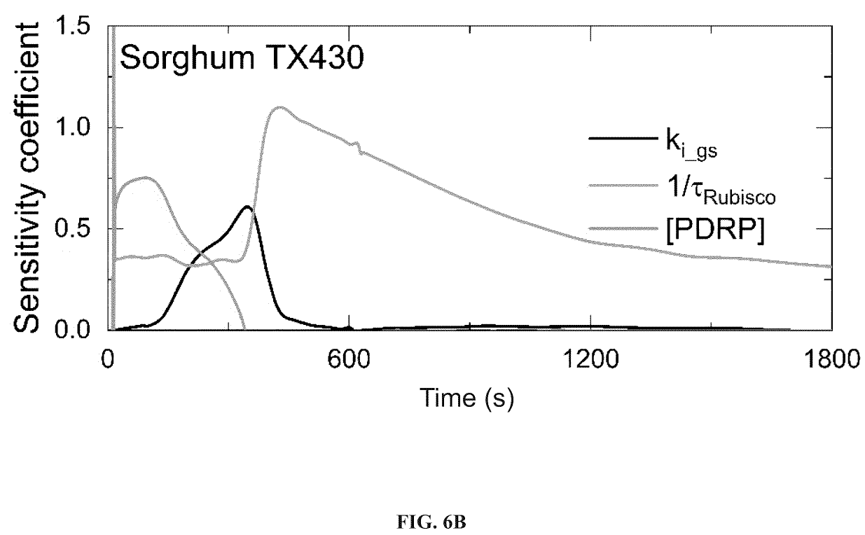

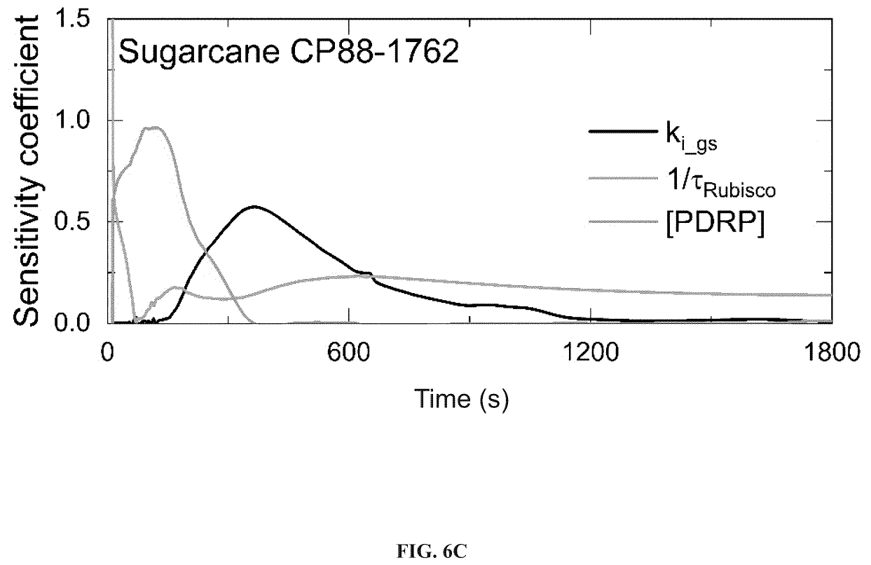

Sensitivity analysis of PDRP and Rca concentrations, as well as the speed of stomatal response indicated that all three limit the rate of induction on dark to light transition in the three C4 crops (FIGS. 6A-6C). However, the strength of each limitations varied between species and time into induction. In maize B73, sensitivity analysis suggested that PDRP exerted the highest limitation in the first 200 s of induction, followed by stomatal opening over the next 400 s and then Rca slightly limited the remaining phase of induction (FIG. 6A). In sorghum Tx430 and sugarcane CP88-1762, the concentration of PDRP limited the rate of induction in the first 240 s, a little longer than that of maize (FIGS. 6B-6C). Rca exerted more influence in sorghum with a peak at around 420 s (FIG. 6B), while the Rca limitation in maize and sugarcane remained approximately constant over this time period (FIGS. 6A and 6C). Stomatal limitation was greater in maize and sugarcane compared to sorghum (FIGS. 6A-6C).

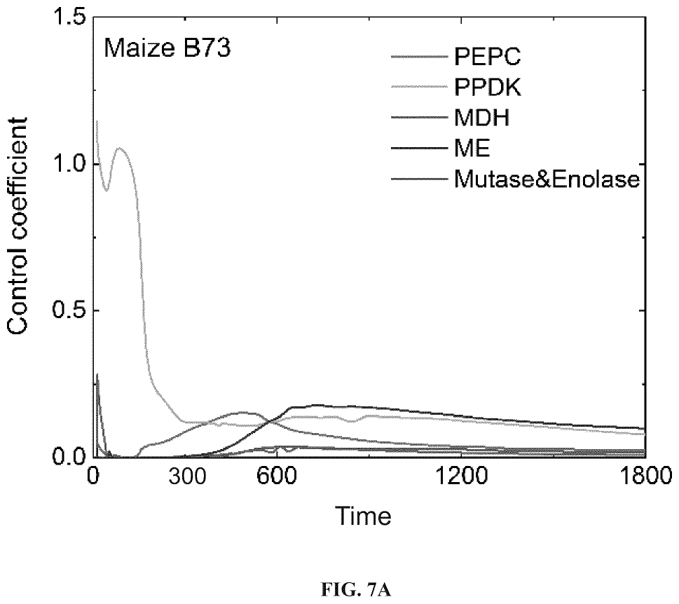

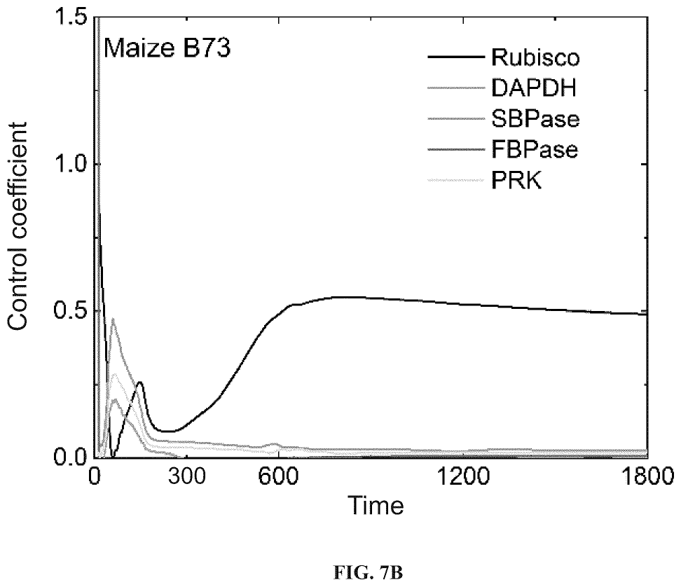

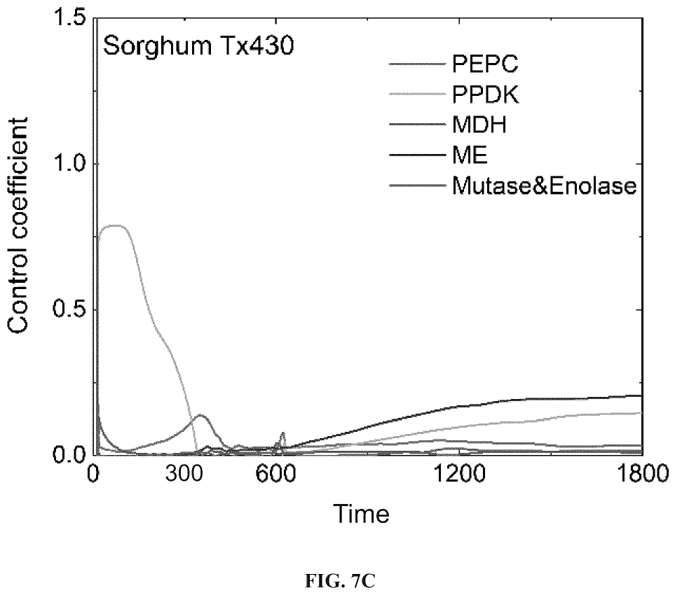

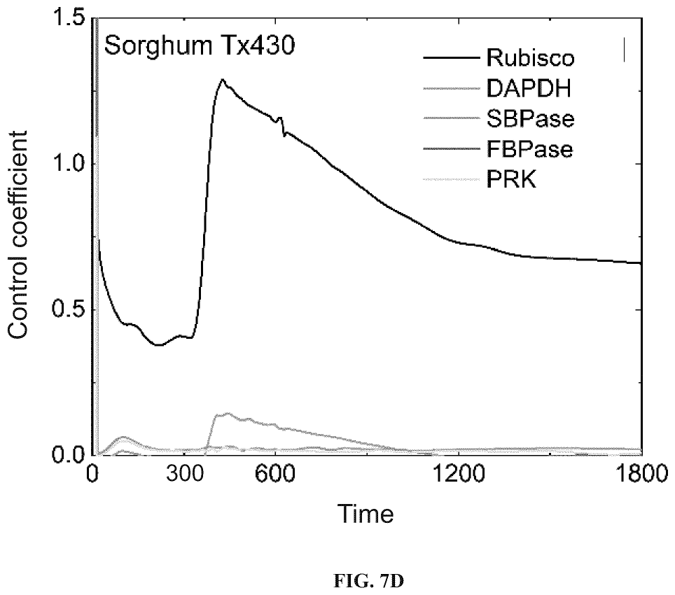

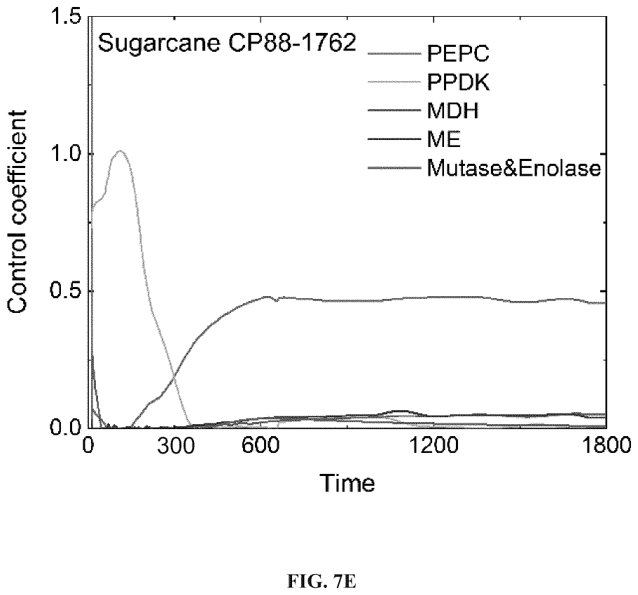

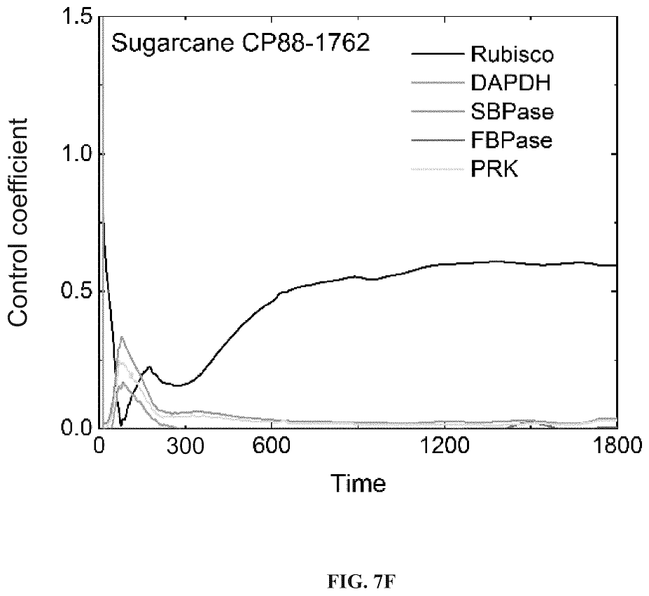

In general, PPDK and Rubisco had high control coefficients in the first few minutes; while that of PPDK then declined, Rubisco continued to exert control through the mid and final stages of the induction. PEPC also had a high control coefficient from the middle of the induction in sugarcane (FIG. 7E). PPDK and ME had some control in the later stage in maize and sorghum (FIG. 7C). A control coefficient states the influence of a single metabolic step on flux through the total pathway, here represented by the rate of CO₂ assimilation. A control coefficient of 1 indicates the step has total control and zero none. Except for Rubisco, other light regulated enzymes of Calvin-Benson cycle, including glyceraldehyde-3-phosphate dehydrogenase (GAPDH), sedoheptulose-bisphosphatase (SBPase) and Phosphoribulokinase (PRK), exerted only mild control in the first 150 s of the induction (FIGS. 7B, 7D, and 7F).

### Predicted CO₂ Leakiness (ϕ) During Photosynthetic Induction

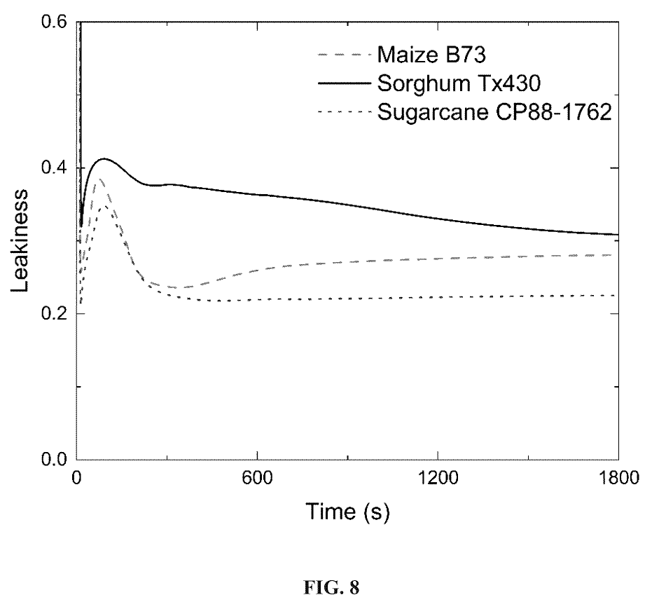

Predicted ϕ showed an increase as PPDK became activated and then declined as simulated Rubisco activity caught up for the three C4 crops. This suggested a loss of co-ordination between the C4 and Calvin-Benson cycles during induction (FIG. 8). Simulated 0 of sorghum declined more slowly than that of maize and sugarcane, due to the slower rate of Rubisco activation (Table 2, FIG. 12).

### DISCUSSION

### Energy Use Efficiency of C4 Leaves was Impacted Under Fluctuating Light

In this example, the average photosynthetic rate through the 30-minute induction was reduced by 18%, 21% and 24% in maize, sorghum and sugarcane, respectively, as compared with the steady-state photosynthetic rate (FIG. 4A). This reduction has a very significant effect on energy use efficiency and net carbon assimilation of crops in the field, since clouds, wind, and the movement of the sun cause frequent light fluctuations within the canopy (Zhu, X. G. et al. (2004) The slow reversibility of photosystem II thermal energy dissipation on transfer from high to low light may cause large losses in carbon gain by crop canopies: a theoretical analysis. Journal of experimental botany, 55, 1167-1175; Kaiser, E. et al. (2018) Fluctuating light takes crop photosynthesis on a rollercoaster ride. Plant Physiology, 176, 977-989; Tanaka, Y. et al. (2019) Natural genetic variation of the photosynthetic induction response to fluctuating light environment. Current opinion in plant biology, 49, 52-59; Wang, Y. et al. (2020) Photosynthesis in the fleeting shadows: an overlooked opportunity for increasing crop productivity? The Plant Journal, 101, 874). In sunny conditions, C4 crops have a higher light energy use efficiency compared to C3 crops due to the CO₂ concentrating mechanism which largely removes the energy cost of photorespiration (Zhu, X.-G. et al. (2010) Improving photosynthetic efficiency for greater yield. Annual review of plant biology, 61, 235-261). However, C4 crops may be less resilient to fluctuating light resulting in a decrease in productivity in dynamic light environment s(Kubasek, J. et al. (2013) C4 plants use fluctuating light less efficiently than dₒ C3 plants: a study of growth, photosynthesis and carbon isotope discrimination. Physiologia plantarum, 149, 528-539). This indicates a significant potential for yield improvement of C4 food and biofuel crops by engineering or breeding for improved speeds of adjustment to fluctuating light.

Including post-translational regulation, the temperature response of enzyme activities, a dynamic stomatal conductance, and a leaf energy balance module, the new dynamic model closely simulated the measured photosynthetic responses of these crops under fluctuating light (FIGS. 5A-5F), in contrast to the original metabolic model (FIGS. 2A-2B). This suggests the model captured the key factors affecting the speed of induction on light fluctuations (FIGS. 5A-5F). Using this model, the factors influencing the speed of induction were determined. With the species-specific input parameters (Table 2), the model was able to predict the limiting factors under conditions of fluctuating light (FIGS. 6A-6C and 7A-7F). This has identified potential targets for energy use efficiency improvement in maize, sorghum and sugarcane. Namely, coordinated up-regulation of Rubisco activase and the PPDK regulatory protein, as well as increased rates of stomatal adjustment.

### Limiting Factors During Photosynthetic Induction.

In C3 plants, the rate of photosynthetic induction is mainly limited by the activation of Rubisco, the activation of the enzymes affecting RuBP regeneration, and the speed of stomata opening, with the major limitations varying between species (McAusland, L. et al. (2016) Effects of kinetics of light-induced stomatal responses on photosynthesis and water—use efficiency. New Phytologist, 211, 1209-1220; Taylor, S. H. and Long, S. P. (2017) Slow induction of photosynthesis on shade to sun transitions in wheat may cost at least 21% of productivity. Philosophical Transactions of the Royal Society B: Biological Sciences, 372, 20160543; Acevedo-Siaca, L. G. et al. (2020) Variation between rice accessions in photosynthetic induction in flag leaves and underlying mechanisms. Journal of experimental botany; Acevedo-Siaca, L. G. et al. (2020) Variation in photosynthetic induction between rice accessions and its potential for improving productivity. New Phytologist; De Souza, A. P. et al. (2020) Photosynthesis across African cassava germplasm is limited by Rubisco and mesophyll conductance at steady state, but by stomatal conductance in fluctuating light. New Phytologist, 225, 2498-2512). However, limitations of C4 photosynthetic efficiency under fluctuating light have received little attention. Through the combination of model simulation and gas exchange experiment, the following limiting factors in photosynthetic induction were identified: 1) accumulation of C4 photosynthetic intermediates to drive intercellular flux; 2) activation of PPDK; 3) stomata opening; and 4) activation of Rubisco.

In the simulations, C4 cycle metabolites took longer to reach a steady-state compared to Calvin-Cycle enzymes (FIGS. 9C-9D), though the influence on the induction of photosynthesis was limited to the first 120 s (FIG. 9A). Also, accelerating the exchange of metabolites between Calvin-Benson cycle and C4 cycle, i.e., increasing the activity of mutase and enolase, which catalyze conversion of PGA to PEP, was able to further reduce the limitation of metabolites during this initial period of the induction (FIGS. 10A-10B). It was noted that mutase and enolase exerted higher control at the beginning of the induction and dropped to zero after about 60 s based on the control analysis for the three C4 crops (FIG. 7A-7F). However, if the leaves experience a short-term sunfleck, increased rates of photosynthetic metabolite accumulation would improve efficiency. The high concentration of C4 metabolites in NADP-ME species at light saturation, results in a slower decline in leaf CO₂ uptake on high-light to shade transitions, as the decarboxylation of malate continues to provide NADPH compensating for some minutes for the decline in NADPH provided from whole chain electron transport (Stitt, M. and Zhu, X. G. (2014) The large pools of metabolites involved in intercellular metabolite shuttles in C4 photosynthesis provide enormous flexibility and robustness in a fluctuating light environment. Plant, cell & environment, 37, 1985-1988).

The results described above infer that increasing the concentration of the PPDK regulatory protein (PDRP) will increase photosynthetic efficiency of these C4 plants under the fluctuating light conditions of field crop canopies. This is based on the simulation using the dynamic model developed in Examples 1 and 2, which suggested that the concentration of PPDK regulatory protein (PDRP) was a major limitation of the induction in the first 180 s for maize and about 250 s for sorghum and sugarcane (FIGS. 6A-6C). It regulates both dark-induced inactivation and light-induced activation of PPDK by catalyzing reversible phosphorylation of a Thr residue (Burnell, J. and Hatch, M. (1983) Dark-light regulation of pyruvate, Pi dikinase in C4 plants: evidence that the same protein catalyses activation and inactivation. Biochemical and biophysical research communications, 111, 288-293; Ashton, A. et al. (1984) Regulation of C4 photosynthesis: inactivation of pyruvate, Pi dikinase by ADP-dependent phosphorylation and activation by phosphorolysis. Archives of Biochemistry and Biophysics, 230, 492-503; Budde, R J. et al. (1985) Studies on the dark/light regulation of maize leaf pyruvate, orthophosphate dikinase by reversible phosphorylation. Archives of Biochemistry and Biophysics, 242, 283-290; Burnell, J. and Hatch, M. (1985) Regulation of C4 photosynthesis: purification and properties of the protein catalyzing ADP-mediated inactivation and Pi-mediated activation of pyruvate, Pi dikinase. Archives of Biochemistry and Biophysics, 237, 490-503; Chastain, C. J. (2010) Structure, function, and post-translational regulation of C 4 pyruvate orthophosphate dikinase. In C4 photosynthesis and related CO₂ concentrating mechanisms: Springer, pp. 301-315; Chastain, C. J. et al. (2018) Maize leaf PPDK regulatory protein isoform-2 is specific to bundle sheath chloroplasts and paradoxically lacks a Pi-dependent PPDK activation activity. Journal of experimental botany, 69, 1171-1181). While these studies elucidated the molecular mechanism of PPDK activation by PDRP, the direct effect of PDRP on photosynthesis had not previously been analyzed. The present analysis suggests over-expression of PDRP would increase photosynthetic efficiency under field conditions.

The sensitivity coefficient of the time constant of stomata opening (kᵢ) indicated that the speed of stomatal opening was rate limiting from 180 s to 600 s after illumination in maize (FIGS. 6A-6C). This differed from the two previous studies which indicated that stomatal conductance was not limiting during the photosynthetic induction in maize because the intercellular CO₂ concentration (G) was always higher than 100 μmol mol⁻¹ during photosynthetic induction (Usuda, H. and Edwards, G. E. (1984) is photosynthesis during the induction period in maize limited by the availability of intercellular carbon dioxide? Plant science letters, 37, 41-45; Furbank, R. and Walker, D. (1985) Photosynthetic induction in C 4 leaves. Planta, 163, 75-83). A meta-analysis of responses of A to Cᵢ across a number of studies of maize indicated that A was only CO₂ saturated at C, >100 μmol mol⁻¹(Pignon, C. P. and Long, S. P. (2020) Retrospective analysis of biochemical limitations to photosynthesis in 49 species; C4 crops appear still adapted to pre-industrial atmospheric [CO₂]. Plant, Cell & Environment, 43, 2606-2622). Here C, dropped as low as 66 μmol mol⁻¹ suggesting that gₛ was a limitation (FIGS. 4A-4E). However, the experiments described in Example 2 used a higher inducing light intensity (1800 μmol m⁻² s⁻¹ as compared to 1400 μmol m⁻² s⁻¹ in Usuda and Edwards (1984) and 115-1150 μmol m² s₋₁ Furbank and Walker (1985) as well as a longer dark treatment time; 30 minutes in comparison to 10 minutes and 20 minutes, respectively. The higher the light intensity used; the lower C, appeared during the induction (Furbank and Walker, 1985). The longer dark treatment time used was to allow sufficient time for stomata to close and Rubisco to de-activate.

The present analysis indicated that activation of Rubisco by Rca is the most important limiting factor after the first few minutes of induction, especially in sorghum with slower Rubisco activation (FIGS. 5A-5F and 6A-6C). In rice, Rca has been demonstrated to play a crucial role in the regulation of non-steady-state photosynthesis (Yamori, W. et al. (2012) Rubisco activase is a key regulator of non-steady-state photosynthesis at any leaf temperature and, to a lesser extent, of steady-state photosynthesis at high temperature. The Plant Journal, 71, 871-880). Rubisco is arguably the major limiting enzyme of light-saturated C4 photosynthesis (von Caemmerer, S. (2000) Biochemical models of leaf photosynthesis: Csiro publishing; von Caemmerer, S. et al. (2005) Reductions of Rubisco activase by antisense RNA in the C4 plant Flaveria bidentis reduces Rubisco carbamylation and leaf photosynthesis. Plant Physiology, 137, 747-755; Kubien, D. S. et al. (2003) C4 photosynthesis at low temperature. A study using transgenic plants with reduced amounts of Rubisco. Plant Physiology, 132, 1577-1585; Wang, Y. et al. (2014) Elements Required for an Efficient NADP-Malic Enzyme Type C4 Photosynthesis. Plant Physiology, 164, 2231-2246) and increasing both Rca and Rubisco content have been shown to increase grain yield in maize (Yin, Z. et al. (2014) Characterization of RuBisCo activase genes in maize: an α-isoform gene functions alongside a β-isoform gene. Plant physiology, 164, 2096-2106; Salesse-Smith, C. E. et al. (2018) Overexpression of Rubisco subunits with RAF1 increases Rubisco content in maize. Nature Plants, 4, 802-810). Hence, based on the simulation described above and the previous studies, increasing the activity of Rubisco and Rca in tandem will increase photosynthetic efficiency in constant and fluctuating light.

PEPC appears not to restrict photosynthesis under steady-state conditions, except under conditions inducing a low Cᵢ, such as drought (Pignon, C. P. and Long, S. P. (2020) Retrospective analysis of biochemical limitations to photosynthesis in 49 species: C4 crops appear still adapted to pre-industrial atmospheric [CO₂]. Plant, Cell & Environment, 43, 2606-2622). However, since Cᵢ dropped below 100 μmol mol⁻¹ during induction (FIG. 4B), sensitivity analysis indicated that increasing PEPC would increase photosynthetic efficiency from 180 s to about 600 s during induction in maize and sorghum (FIGS. 7A and 7C). In sugarcane, however, PEPC limits steady-state photosynthetic rate of sugarcane, due to its lower Vₚₘₐₓ compared to maize and sorghum (FIG. 7E, Table 2).

### Differences in the Limiting Factors of Photosynthetic Induction Among Species

Furbank et al. (Furbank, R. T. et al. (1997) Genetic manipulation of key photosynthetic enzymes in the C-4 plant Flaveria bidentis. Australian Journal of Plant Physiology, 24, 477-485) concluded from anti-sense manipulations that PPDK and Rubisco shared metabolic control of steady-state light-saturated photosynthesis in the C4 dicot Flaveria bidentis. The limited studies of C4 photosynthesis under fluctuating light have focused on maize. Two early studies indicated that photosynthesis reached a maximum rate after about 15-25 min in maize (Usuda, H. and Edwards, G. E. (1984) Is photosynthesis during the induction period in maize limited by the availability of intercellular carbon dioxide? Plant science letters, 37, 41-45; Furbank, R and Walker, D. (1985) Photosynthetic induction in C 4 leaves. Planta, 163, 75-83), which is comparable with measurements described herein (FIGS. 5A-5F and 4A).

The above experiments were limited to single accessions of three NADP-ME C4 species. Therefore, the results cannot be generalized to the species. However, examination of individuals from three distinct species of the monophyletic Andropogonae, all C4-NADP-ME plants, has likely revealed limitations that apply across this key clade of food and energy crops. They therefore point to manipulations that could improve photosynthetic efficiency and yields across the clade. Although there were many similarities, some differences were found. Maize, as perhaps the species most intensively bred for productivity, showed the fastest induction and greatest efficiency of carbon gain over the period of induction, while sugarcane was the slowest (FIGS. 4A-4E). Whether these are species characteristics could only be determined by analyzing a wider range of genotypes of each crop. Characterizing within species variation would also show the potential for improving non-steady-state photosynthesis through breeding. In rice, intra-specific genetic variation in non-steady-state photosynthetic efficiency was found to be substantially greater than in steady-state, suggesting an overlooked target for improvement, that might similarly be available in these crops (Acevedo-Siaca, L. G. et al. (2020) Variation between rice accessions in photosynthetic induction in flag leaves and underlying mechanisms. Journal of experimental botany; Acevedo-Siaca, L. G. et al. (2020) Variation in photosynthetic induction between rice accessions and its potential for improving productivity. New Phytologist).

Maize showed the fastest induction, because of more PDRP and faster τ_{Rubisco} (Table 2), which indicated that maize has faster PPDK and Rubisco activation capacity. However, the stomatal response of maize was slow (Table 2). Here the stomata were one of the major limiting factors during the induction process (FIGS. 6A-6C). This conclusion was different from previous studies (Usuda, H. and Edwards, G. E. (1984) Is photosynthesis during the induction period in maize limited by the availability of intercellular carbon dioxide? Plant science letters, 37, 41-45; Furbank, R and Walker, D. (1985) Photosynthetic induction in C 4 leaves. Planta, 163, 75-83), and the possible reasons were explained in the previous section. Speeding stomatal opening and closing is the key to speed up photosynthetic response while maintaining water use efficiency. New combined thermal and modulated fluorescence techniques now provide a potential high-throughput means to screen germplasm for this trait (Vialet-Chabrand, S. and Lawson, T. (2019) Dynamic leaf energy balance: deriving stomatal conductance from thermal imaging in a dynamic environment. Journal of experimental botany, 70, 2839-2855; Vialet-Chabrand, S. and Lawson, T. (2020) Thermography methods to assess stomatal behaviour in a dynamic environment. Journal of experimental botany, 71, 2329-2338). Bioengineering for more and smaller stomatal complexes would be another approach (Drake, P. L. et al. (2013) Smaller, faster stomata: scaling of stomatal size, rate of response, and stomatal conductance. Journal of Experimental Botany, 64, 495-505).

For sorghum, the speed of stomatal opening had little effect on A during induction (FIGS. 6A-6C). Enzyme activities were the main limiting factors, i.e. the concentration of [PDRP] (the activation rate of PPDK), the activation rate of Rubisco (τ_{Rubisco}) and Rubisco activity (V_{cmax}) (FIGS. 6A-6C and 7A-7F). Thus, increasing the activity of PDRP, Rca and Rubisco would lead to higher dynamic photosynthesis. However, analysis of water use efficiency across a wide range of sorghum germplasm suggests at the species level speed of stomatal adjustment is also important (Pignon, C. P. et al. (2021) Drivers of Natural Variation in Water-Use Efficiency Under Fluctuating Light Are Promising Targets for Improvement in Sorghum. Frontiers in Plant Science, 12, 627432).

For sugarcane, its dynamic photosynthetic efficiency was co-limited by many factors, including the rate of stomata opening, the concentration of [PDRP], the activation rate of Rubisco(τ_{Rubisco}) and Rubisco activity (V_{cmax}). In addition, a high control coefficient of PEPC, relative to the other species, was found in sugarcane during induction and at steady-state (FIG. 7A-7E). Therefore, to improve dynamic photosynthesis, all the limiting factors above should be considered comprehensively.

### Imbalances in the Regulation of C3 and C4 Cycles

Coordination between the C3 and the C4 cycle is essential to efficiency of C4 photosynthesis. Leakiness (#) describes the proportion of carbon fixed by PEP carboxylation that retrodiffuses back out of bundle-sheath cells into the mesophyll (Eq. 30). It was estimated to be about 0.2 in several C4 species under various environmental conditions (Henderson, S. A., Caemmerer, S. and Farquhar, G. D. (1992) Short-term measurements of carbon isotope discrimination in several C4 species. Functional Plant Biology, 19, 263-285) and between 0.20-0.22 in a recent study of maize (Salesse-Smith, C. E. et al. (2018) Overexpression of Rubisco subunits with RAF1 increases Rubisco content in maize. Nature Plants, 4, 802-810). The simulated steady-state ϕ of the three species was between 0.2-0.3 (FIG. 8). In the simulation, the leakiness was predicted to change during the induction, due to an imbalance in the regulation of Calvin-Benson cycle and C4 cycles, especially when the activation of Rubisco was slower (FIG. 8; Sorghum). Overall, this disclosure has identified several potential opportunities for increasing photosynthetic efficiency in these major crops during the frequent light fluctuations that occur in field canopies.

### Example 4. Genetically Altered Sorghum bicolor (Sorghum)

This disclosure also provides for a genetically altered Sorghum bicolor (sorghum) line with increased activity of a PDK regulatory protein (PDRP), a Rubisco activase (Rca) protein, and/or a Rubisco protein as compared to a wild type plant grown under the same conditions (e.g., non-steady light, field conditions, fluctuating light). This increased activity results in increased photosynthetic efficiency, yield, and/or water use efficiency as compared to the wild type plant grown under the same conditions.

The sorghum line can be created by generating a population of transgenic plants comprising heterologous nucleotide sequences encoding polypeptides selected from PDRP, Rca, or Rubisco; a combination of two or more of PDRP, Rca, or Rubisco; and/or all three of PDRP, Rca, and Rubisco, as described herein. Each transgenic event comprises introducing into the genome of a parent plant at least one nucleotide construct comprising a promoter operably linked to a heterologous nucleotide as described herein. The nucleotide construct is introduced into the parental genome in sufficient quantity to produce transgenic cells which can be cultured into plants of transgenic sorghum having said enhanced phenotype. The transgenic cells are cultured into transgenic plants producing progeny transgenic seed. The population of transgenic plants is screened for observable phenotypes. Seed is collected from transgenic plants which are selected as having an unexpected enhanced phenotype. Optionally, the method includes repeating a cycle of germinating transgenic seed, growing subsequent generation plants from said transgenic seed, observing phenotypes of said subsequent generation plants and collecting seeds from subsequent generation plants having an enhanced phenotype. In another aspect, the method includes a large population being screened for at least one heterologous nucleotide sequences encoding polypeptides selected from PDRP, Rca, or Rubisco. Other aspects of the method employ nucleotide constructs where the heterologous DNA is operably linked to a selected promoter, e.g. the 5′ end of a promoter region. The DNA construct may be introduced into a random location in the genome or into a preselected site in the genome.

Yet further aspects employ genome editing methods to introduce genetic alterations into sorghum that increase activity of PDRP, Rca, and/or Rubisco by targeting a nuclear genome sequence operably linked to an endogenous PDRP, Rca, and/or Rubisco protein. These genome editing methods may include gene editing components including a ribonucleoprotein complex, a TALEN protein, a ZFN protein, an oligonucleotide donor, and/or a CRISPR/Cas enzyme.

Examples of sorghum transformation protocols are described in Guo et al., Methods Mol Biol 1223, 181-188, 2015, as well as Howe et al., Plant Cell Rep 25(8): 784-791, 2006.

### Example 5. Genetically Altered Zea mays (Corn)

This disclosure also provides for a genetically altered Zea mays (corn) line with increased activity of a PDK regulatory protein (PDRP), a Rubisco activase (Rca) protein, and/or a Rubisco protein as compared to a wild type plant grown under the same conditions (e.g., non-steady light, field conditions, fluctuating light). This increased activity results in increased photosynthetic efficiency, yield, and/or water use efficiency as compared to the wild type plant grown under the same conditions.

The corn line can be created by generating a population of transgenic plants comprising heterologous nucleotide sequences encoding polypeptides selected from PDRP, Rca, or Rubisco; a combination of two or more of PDRP, Rca, or Rubisco; and/or all three of PDRP, Rca, and Rubisco, as described herein. Each transgenic event comprises introducing into the genome of a parent plant at least one nucleotide construct comprising a promoter operably linked to a heterologous nucleotide as described herein. The nucleotide construct is introduced into the parental genome in sufficient quantity to produce transgenic cells which can be cultured into plants of transgenic sorghum having said enhanced phenotype. The transgenic cells are cultured into transgenic plants producing progeny transgenic seed. The population of transgenic plants is screened for observable phenotypes. Seed is collected from transgenic plants which are selected as having an unexpected enhanced phenotype. Optionally, the method includes repeating a cycle of germinating transgenic seed, growing subsequent generation plants from said transgenic seed, observing phenotypes of said subsequent generation plants and collecting seeds from subsequent generation plants having an enhanced phenotype. In another aspect, the method includes a large population being screened for at least one heterologous nucleotide sequences encoding polypeptides selected from PDRP, Rca, or Rubisco. Other aspects of the method employ nucleotide constructs where the heterologous DNA is operably linked to a selected promoter, e.g. the 5′ end of a promoter region. The DNA construct may be introduced into a random location in the genome or into a preselected site in the genome.

Yet further aspects employ genome editing methods to introduce genetic alterations into corn that increase activity of PDRP, Rca, and/or Rubisco by targeting a nuclear genome sequence operably linked to an endogenous PDRP, Rca, and/or Rubisco protein. These genome editing methods may include gene editing components including a ribonucleoprotein complex, a TALEN protein, a ZFN protein, an oligonucleotide donor, and/or a CRISPR/Cas enzyme.

Examples of corn transformation protocols are described in Yassitepe JEdCT, da Silva VCH, Hernandes-Lopes J, Dante RA, Gerhardt IR, Fernandes FR, da Silva PA, Vieira LR, Bonatti V and Arruda P(2021) Maize Transformation: From Plant Material to the Release of Genetically Modified and Edited Varieties. Front. Plant Sci. 12:766702, as well as Kausch, A. P., Wang, K., Kaeppler, H. F. et al. Maize transformation: history, progress, and perspectives. Mol Breeding 41, 38 (2021).

### Example 6. Genetically Altered Saccharum Oficinarum (Sugarcane)

This disclosure also provides for a genetically altered Saccharum officinarum (sugarcane) line with increased activity of a PDK regulatory protein (PDRP), a Rubisco activase (Rca) protein, and/or a Rubisco protein as compared to a wild type plant grown under the same conditions (e.g., non-steady light, field conditions, fluctuating light). This increased activity results in increased photosynthetic efficiency, yield, and/or water use efficiency as compared to the wild type plant grown under the same conditions.

The sugarcane line can be created by generating a population of transgenic plants comprising heterologous nucleotide sequences encoding polypeptides selected from PDRP, Rca, or Rubisco; a combination of two or more of PDRP, Rca, or Rubisco; and/or all three of PDRP, Rca, and Rubisco, as described herein. Each transgenic event comprises introducing into the genome of a parent plant at least one nucleotide construct comprising a promoter operably linked to a heterologous nucleotide as described herein. The nucleotide construct is introduced into the parental genome in sufficient quantity to produce transgenic cells which can be cultured into plants of transgenic sorghum having said enhanced phenotype. The transgenic cells are cultured into transgenic plants producing progeny transgenic seed. The population of transgenic plants is screened for observable phenotypes. Seed is collected from transgenic plants which are selected as having an unexpected enhanced phenotype. Optionally, the method includes repeating a cycle of germinating transgenic seed, growing subsequent generation plants from said transgenic seed, observing phenotypes of said subsequent generation plants and collecting seeds from subsequent generation plants having an enhanced phenotype. In another aspect, the method includes a large population being screened for at least one heterologous nucleotide sequences encoding polypeptides selected from PDRP, Rca, or Rubisco. Other aspects of the method employ nucleotide constructs where the heterologous DNA is operably linked to a selected promoter, e.g. the 5′ end of a promoter region. The DNA construct may be introduced into a random location in the genome or into a preselected site in the genome.

Yet further aspects employ genome editing methods to introduce genetic alterations into sugarcane that increase activity of PDRP, Rca, and/or Rubisco by targeting a nuclear genome sequence operably linked to an endogenous PDRP, Rca, and/or Rubisco protein. These genome editing methods may include gene editing components including a ribonucleoprotein complex, a TALEN protein, a ZFN protein, an oligonucleotide donor, and/or a CRISPR/Cas enzyme.

Examples of sugarcane transformation protocols are described in Radhesh Krishnan, S., Mohan, C. (2017). Methods of Sugarcane Transformation. In: Mohan, C. (eds) Sugarcane Biotechnology: Challenges and Prospects. Springer, Cham., as well as Budeguer F, Enrique R, Perera MF, Racedo J, Castagnaro AP, Noguera AS and Welin B(2021) Genetic Transformation of Sugarcane, Current Status and Future Prospects. Front. Plant Sci. 12:768609. doi: 10.3389/fpls.2021.768609.

1: A genetically altered plant or plant part comprising one or more first genetic alterations that increase activity of a PPDK regulatory protein (PDRP) as compared to a wild type plant or plant part grown under the same conditions, wherein the genetically altered plant is a C4 plant, and optionally further comprising one or more second genetic alterations that increase activity of a Rubisco activase (Rca) protein and/or a Rubisco protein, as compared to a wild type plant or plant part grown under the same conditions.

2: A genetically altered plant or plant part comprising one or more first genetic alterations that increase activity of a Rubisco activase (Rca) protein and/or a Rubisco protein as compared to a wild type plant or plant part grown under the same conditions, wherein the genetically altered plant is a C4 plant, and optionally further comprising one or more first genetic alterations that increase activity of the PDRP protein, as compared to the wild type plant or plant part grown under the same conditions, and further comprising one or more second genetic alterations that increase activity of the Rca protein, as compared to the wild type plant or plant part grown under the same conditions.

3: The genetically altered plant or plant part of claim 1, wherein the PDRP protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 1, SEQ ID NO: 2, SEQ ID NO: 3, SEQ ID NO: 14, SEQ ID NO: 15, SEQ ID NO: 16, or SEQ ID NO: 17; and/or the Rca protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 4, SEQ ID NO: 5, SEQ ID NO: 6, or SEQ ID NO: 7; and/or the Rubisco protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 8, SEQ ID NO: 9, SEQ ID NO: 10, SEQ ID NO: 11, SEQ ID NO: 12, or SEQ ID NO: 13.

4: The genetically altered plant or plant part of claim 1, further comprising one or more third genetic alterations that increase a speed of stomatal opening and closing, as compared to a wild type plant or plant part grown under the same conditions.

5: The genetically altered plant or plant part of claim 1, further comprising one or more fourth genetic alterations that increase a number of stomatal complexes and one or more fifth genetic alterations that decrease a size of stomatal complexes, as compared to a wild type plant or plant part grown under the same conditions.

6: The genetically altered plant or plant part of claim 1, wherein the one or more first genetic alterations, one or more second genetic alterations, one or more third genetic alterations, one or more fourth genetic alterations, and one or more fifth genetic alterations that increase activity comprise overexpression, and wherein the overexpression is due to a transgene overexpressing a protein with the activity being increased and/or the overexpression is due to genetic alterations in a promoter of an endogenous gene for the protein with the activity being increased.

7: The genetically altered plant or plant part of claim 1, wherein the growth conditions comprise non-steady light, optionally field conditions or fluctuating light, and wherein the genetically altered plant or plant part has increased photosynthetic efficiency, yield, and/or water use efficiency as compared to a wild type plant or plant part grown under the same conditions.

8: The genetically altered plant or plant part of claim 1, wherein the plant is Zea mays, Saccharum oficinarum, or Sorghum bicolor.

9: The genetically altered plant or plant part of claim 1, further comprising one or more sixth genetic alterations that increase activity of PEPC, as compared to a wild type plant or plant part grown under the same conditions.

10: A method of producing the genetically altered plant or plant part of claim 1, comprising:
a) introducing the one or more first genetic alterations that increase activity of the PDRP protein, the one or more second genetic alterations that increase activity of the Rca protein and/or the Rubisco protein, or both the one or more first genetic alterations that increase activity of the PDRP protein and the one or more second genetic alterations that increase activity of the Rca protein and/or the Rubisco protein into a plant cell, tissue, or other explant of a C4 plant;
b) regenerating the plant cell, tissue, or other explant into a genetically altered C4 plantlet; and
c) growing the genetically altered C4 plantlet into a genetically altered C4 plant with the one or more genetic alterations that increase activity of the PDRP protein, the one or more genetic alterations that increase activity of the Rca protein and/or the Rubisco protein, or both the one or more genetic alterations that increase activity of the PDRP protein and the one or more genetic alterations that increase activity of the Rca protein and/or the Rubisco protein.

11: The method of claim 10, wherein introducing the one or more genetic alterations that increase activity of the PDRP protein comprises transforming a plant cell, tissue, or other explant of a C4 plant with a first vector comprising a first nucleic acid sequence encoding the PDRP protein operably linked to a second nucleic acid sequence encoding a first promoter, and/or wherein introducing the one or more genetic alterations that increase activity of the Rca protein and/or the Rubisco protein comprises transforming a plant cell, tissue, or other explant of a C4 plant with a second vector comprising a second nucleic acid sequence encoding the Rca protein operably linked to a second promoter and/or a third vector comprising a third nucleic acid sequence encoding the Rubisco protein operably linked to a third promoter, optionally wherein the first vector, the second vector, and/or the third vector are introduced as a single nucleic acid construct or the first vector, the second vector, and/or the third vector are introduced separately, optionally wherein the separate introduction is into different C4 plants or C4 plant parts and the first vector, the second vector, and/or the third vector are combined through crossing the different C4 plants.

12: The method of claim 11, wherein the first promoter, the second promoter, and the third promoter, are selected from the group of a constitutive promoter, an inducible promoter, a tissue or cell type specific promoter, and an inducible, tissue or cell type specific promoter.

13: The method of claim 10, wherein introducing the one or more genetic alterations that increase activity of the PDRP protein comprises transforming a plant cell, tissue, or other explant of a C4 plant with one or more first gene editing components that target a nuclear genome sequence operably linked to an endogenous PDRP protein, and/or wherein introducing the one or more genetic alterations that increase activity of the Rca protein and the Rubisco protein comprises transforming a plant cell, tissue, or other explant of a C4 plant with one or more second gene editing components that target a nuclear genome sequence operably linked to an endogenous Rca protein and one or more third gene editing components that target a nuclear genome sequence operably linked to an endogenous Rubisco protein.

14: The method of claim 13, wherein the one or more first gene editing components, the one or more second gene editing components, and the one or more third gene editing components, comprise a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZFN protein targets the nuclear genome sequence; an oligonucleotide donor (OND), wherein the OND targets the nuclear genome sequence; or a vector CRISPR/Cas enzyme encoding sequence and a targeting sequence, wherein the targeting sequence targets the nuclear genome sequence.

15: The method of claim 10, further comprising introducing one or more third genetic alterations that increase a speed of stomatal opening and closing, as compared to a wild type plant or plant part grown under the same conditions; introducing one or more fourth genetic alterations that increase a number of stomatal complexes and one or more fifth genetic alterations that decrease a size of stomatal complexes, as compared to a wild type plant or plant part grown under the same conditions; and/or introducing one or more sixth genetic alterations that increase activity of a PEPC protein, as compared to a wild type plant or plant part grown under the same conditions.

16: The method of claim 10, wherein the plant is Zea mays, Saccharum oficinarum, or Sorghum bicolor.

17: A genetically altered plant produced by the method of claim 10, wherein the genetically altered plant has increased photosynthetic efficiency, increased yield potential, and/or increased water use efficiency as compared to a wild type plant or plant part grown under the same conditions.

18: A method of cultivating a genetically altered plant with increased photosynthetic efficiency, comprising the steps of:
a) providing the genetically altered plant, wherein the plant or a part thereof comprises one or more genetic alterations, and wherein the plant is a C4 plant; and
b) cultivating the genetically altered plant under conditions wherein the one or more genetic alterations increase activity of a PPDK regulatory protein (PDRP), as compared to a wild type plant grown under the same conditions, increase activity of a Rubisco activase (Rca) protein and/or a Rubisco protein comprising, as compared to a wild type plant grown under the same conditions, or increase activity of a PDRP protein and a Rca protein and/or a Rubisco protein, as compared to a wild type plant grown under the same conditions, and wherein the increased activity of the PDRP protein, the Rca protein, and/or the Rubisco protein increases photosynthetic efficiency in the genetically altered plant as compared to the wild type plant grown under the same conditions.

19: The method of claim 18, wherein the PDRP protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 1, SEQ ID NO: 2, SEQ ID NO: 3, SEQ ID NO: 14, SEQ ID NO: 15, SEQ ID NO: 16, or SEQ ID NO: 17; and/or the Rca protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 100% sequence identity to one of SEQ ID NO: 4, SEQ ID NO: 5, SEQ ID NO: 6, or SEQ ID NO: 7; and/or the Rubisco protein comprises an amino acid sequence having at least 70% sequence identity, at least 75% sequence identity, at least 80% sequence identity, at least 85% sequence identity, at least 90% sequence identity, at least 91% sequence identity, at least 92% sequence identity, at least 93% sequence identity, at least 94% sequence identity, at least 95% sequence identity, at least 96% sequence identity, at least 97% sequence identity, at least 98% sequence identity, at least 99% sequence identity, or 1⁰⁰% sequence identity to one of SEQ ID NO: 8, SEQ ID NO: 9, SEQ ID NO: 10, SEQ ID NO: 11, SEQ ID NO: 12, or SEQ ID NO: 13.

20: The method of claim 18, wherein the conditions comprise non-steady light, optionally field conditions or fluctuating light.

21: The method of claim 18, wherein the genetically altered plant further comprises increased yield as compared to the wild type plant grown under the same conditions.

22: An isolated DNA molecule comprising the first vector, the second vector, and/or the third vector of claim 11.
